(19) United States
(12) Patent Application Publication
(10) Pub. No.: US 2013/0006130 A1
(43) Pub. Date: Jan. 3, 2013

(54) DEVICE AND METHOD FOR MONITORING A FLUID FLOW RATE IN A CARDIOVASCULAR SYSTEM

(75) Inventors: Bo Olde, Lund (SE); Kristian Solem, Kavlinge (SE)

(73) Assignee: Gambro Lundia AB, Lund (SE)

(21) Appl. No.: 13/519,483

(22) PCT Filed: Dec. 22, 2010
(86) PCT No.: PCT/EP2010/070565

§ 371 Date: Sep. 13, 2012

Related U.S. Application Data
(60) Provisional application No. 61/290,319, filed on Dec. 28, 2009.

(30) Foreign Application Priority Data
Dec. 28, 2009 (SE) 0951030-6

(51) Int. Cl. A61B 5/02

(52) U.S. Cl. 600/504

(57) ABSTRACT

A device in an apparatus for extracorporeal blood treatment is configured to monitor a fluid flow rate (Q) of a cardiovascular system of a subject. The apparatus comprises an extracorporeal blood circuit and a connection (C) for connecting the extracorporeal blood circuit to the cardiovascular system. The device comprises an input for obtaining a time-dependent measurement signal (d(n)) from a pressure sensor in the extracorporeal blood circuit. The pressure sensor is arranged to detect a subject pulse originating from a subject pulse generator in the cardiovascular system of the subject, wherein the system further comprises a signal processor connected to the input. The signal processor is configured to process the measurement signal to obtain a pulse profile (e(n)) which is a temporal signal profile of the subject pulse, and to calculate a fluid flow rate (Q) based at least partly on the temporal signal profile.

### TECHNICAL FIELD

The present invention relates to a device and method for monitoring fluid flow rates in a subject's cardio-vascular system. More particularly, the invention relates to the calculation of flow rates in the cardio-vascular system based on temporal signal profiles of pulses originating from a pulse generator of the subject.

### BACKGROUND ART

In extracorporeal blood treatment, blood is taken out of a subject, treated and then reintroduced into the subject by means of an extracorporeal blood flow circuit. Generally, the blood is circulated through the circuit by one or more pumping devices. The circuit is connected to a blood vessel access of the patient, typically via one or more access devices, such as needles, which are inserted into the blood vessel access. Such extracorporeal blood treatments include hemodialysis, hemodiafiltration, hemofiltration, plasmapheresis, bloodbanking, blood fraction separation (e.g. cells) of donor blood, etc.

In hemodialysis and similar treatments, a blood access is commonly surgically created in the nature of an arterio-venous (AV) shunt, commonly referred to as a fistula. Blood needles or catheters are inserted into the fistula. Blood is taken out from the fistula via a needle or catheter at an upstream position and blood is returned to the fistula via a needle or catheter at a downstream position. The arterio-venous shunt or fistula provides a blood access having capability of providing a high blood flow and being operative during several years and even tens of years. It is produced by connecting, for example, the radial artery to the cephalic vein at the level of the forearm. The venous limb of the fistula thickens during the course of several months, permitting repeated insertion of dialysis needles or catheters. Alternative blood accesses to the fistula are for instance an arterio-venous graft or a silicon, dual-lumen catheter surgically implanted into one of the large veins. Other methods and devices are also known.

During the above blood treatment therapies, hemodialysis for instance, it is desirable to obtain a constant blood flow rate of 150-500 ml/min or even higher, and the access site must be prepared for delivering such flow rates. The blood flow in an AV fistula is often 800 ml/min or larger, permitting delivery of a blood flow rate in the desired range.

In the absence of a sufficient treated blood entering the fistula via the venous needle, the extracorporeal circuit blood pump will take up some of the already treated blood entering the fistula via the venous needle, so called access or fistula recirculation, leading to poor treatment results and progressive reduction of treatment efficiency.

A common cause of poor flow with AV fistulas is partial obstruction of the venous limb due to fibrosis secondary to multiple veinpunctures. Moreover, stenosis causes a reduction of access flow.

It is known that access flow rate often exhibits a long plateau time period with sufficient access flow, followed by a short period of a few weeks with markedly reduced access flow leading to recirculation and ultimately access failure. By monitoring the evolution of the access flow during consecutive treatment sessions, it is possible to detect imminent access flow problems. Proper detection of access flow reduction may help in carrying out a maintenance procedure on the access thereby avoiding any access failure.

It is known in the art to measure different parameters of a cardiovascular system. In particular, methods are known for detection of parameters relating to the access, such as access flow, or to cardiac output.

Monitoring of parameters such as access flow of a blood access such as a fistula may be performed by ultrasound (Doppler) measurements. Alternatively, dilution methods may be used to obtain access flow by measuring a difference in conductivity created by a reversal of flow directions of blood to and from the blood access.

However, known methods for determining flow-related parameters during dialysis or blood treatment require separate, specialised instruments and sensors, and are not suited for repeated or continuous, beat-to-beat measurements and monitoring.

In particular, the known methods comprise isolated measurements, and are not well-suited for continuous intra-dialytic monitoring of cardiac output and access flow.

Enhanced monitoring of cardio-vascular flows may provide numerous benefits, in particular in connection with extracorporeal treatments. For instance, monitoring of cardiac output would be beneficial in connection with dialysis since water removal, i.e. ultrafiltration, during dialysis may reduce cardiac output, which may lead to an increased risk for the subject undergoing the treatment to suffer from hypotension. The reason is that cardiac output depends on the venous blood flow returning to the heart, which in turn may decrease as the total blood volume decreases after running ultrafiltration at higher rate compared to the vascular refill rate.

Access flow measurements are important for the clinician to determine if a blood access of a dialysis patient can provide sufficient blood flow to allow adequate dialysis treatment. Normally, access flow measurements are conducted regularly, e.g. once a month, in order to detect low values or declining trends. Such indications may urge the physician to perform an access intervention for instance surgery to alleviate the situation.

Hence, there is a need for monitoring various cardiovascular flows, in particular during extracorporeal treatment such as dialysis.

Continuous cardiac output measurement may be important in adjusting the ultrafiltration rate properly to reduce the risk for hypotension.

In addition, cardiac output variations between treatments or over longer periods may be an indication of a heart condition, which may call for further medical investigation.

### SUMMARY OF THE INVENTION

It is an object of the invention to at least partly overcome one or more of the above-identified limitations of the prior art and in particular to provide an alternative or complementary technique for monitoring fluid flow rates in a subject's cardio-vascular system. Specifically, it is an object to provide calculations of flow rates in the cardio-vascular system based on temporal signal profiles of pulses originating from a pulse generator of the subject.

This and other objects, which will appear from the description below, are at least partly achieved by means of devices for monitoring, a method for monitoring, and a computer program product according to the independent claims, embodiments thereof being defined by the dependent claims.

A first aspect of the invention is a device for monitoring a fluid flow rate of a cardiovascular system of a subject, said device comprising an input for obtaining a time-dependent measurement signal from a pressure sensor in an extracorporeal blood circuit which is adapted for connection to the cardiovascular system, the pressure sensor being arranged to detect a subject pulse originating from a subject pulse generator, wherein the device further comprises a signal processor connected to the input and being configured to: process the measurement signal to obtain a pulse profile which is a temporal signal profile of the subject pulse, and calculate a fluid flow rate based at least partly on the temporal signal profile.

In one embodiment, the pulse generator is a part of the cardiovascular system. Hence, requiring no external source.

In one embodiment, the pulse generator is any of the heart, the breathing system, or any combinations thereof. Hence, the pulse generator is part of the cardiovascular system.

In one embodiment, the extracorporeal blood circuit comprises a fluid pathway, a blood processing device, and at least one pumping device, and wherein the pressure sensor is further arranged to detect a pump pulse originating from the pumping device.

In one embodiment, the calculating of a fluid flow rate (Q) involves a pulse parameter P from one or more of amplitude, shape, and timing of the temporal signal profile. The pulse parameter P may also be referred to as a pulse feature.

A second aspect of the invention is a method for monitoring a fluid flow rate in a cardiovascular system of a subject, said method comprising: obtaining a time-dependent measurement signal from a pressure sensor in an extracorporeal blood circuit which is arranged in fluid connection with the cardiovascular system, the pressure sensor being arranged to detect a subject pulse originating from a subject pulse generator; processing the measurement signal to obtain a pulse profile which is a temporal signal profile of the subject pulse, and calculating a fluid flow rate based at least partly on the temporal signal profile.

In one embodiment, the method further comprises varying a blood flow of the extracorporeal circuit. Hence, by varying the blood flow in an extracorporeal circuit of for instance a dialysis monitor to cause a perturbation to the hydraulic system in the fistula such that the dynamics in the hydraulic system is altered and thus increasing the number of relationships for determining the number of unknown variables. The variation may be caused by for instance varying the direction and/or magnitude of the blood flow.

In one embodiment, the method further comprises aggregating a plurality of subject pulse profiles within an aggregation time window in the measurement signal and calculating the fluid flow rate based on an average of the plurality of the subject pulse profiles.

In one embodiment, the calculating involves calculation of the cardiac output of the cardiovascular system. An alarm event may be generated when the Cardiac Output (CO) exceeds a predetermined threshold.

In one embodiment, the calculating involves calculation of an access flow of a blood access in the cardiovascular system.

In one embodiment, the method further comprises calibrating the fluid flow rate against one or more calibration values. For instance, the method may be calibrated with one or more reference values at the beginning of and/or prior to a treatment, at the end of a treatment and/or during a treatment.

In one embodiment, the method further comprises calculating an average access flow rate Qa and an associated variability QaV, retrieving a withdrawal blood flow rate Qb, and in case the sum of blood flow rate Qb and access flow variance QaV exceeds Qa, generating an alarm event. Hence, recirculation may be detected. The variability QaV may comprise e.g. standard deviation or variance. Alternatively, an alarm event may be generated in a case when the withdrawal blood flow rate Qb exceeds a momentary access flow rate Qa.

In one embodiment, the method further comprises calculating at least one additional fluid flow rate (Qx), calculating an average fluid flow rate (Qavg) determined from the calculated fluid flow rate (Q) and the at least one additional fluid flow rate (Qx), calculating an average reference fluid flow rate (Qavg_ref), and adjusting the average reference fluid flow rate (Qavt_ref) based on the fluid flow rate (Q) and the at least one additional fluid flow rate (Qx). Hence, reference measurements of average fluid flow rates may be corrected by taking into account the variance of the fluid flow rate, i.e. fluctuations or periodical variations.

In one embodiment, the calibration comprises: providing a detectable perturbation to at least a measurable blood characteristic in the cardiovascular system; measuring an integrated change of a corresponding characteristic on a treatment fluid outlet of the extracorporeal blood circuit; and determining the cardiovascular flow rate based on the measurement of said integrated change of the treatment fluid outlet. For instance, the detectable perturbation may be in the form of a pulse. The perturbation may be a change in concentration of a detectable substance such as urea, salt or salt in the form of a saline solution.

In one embodiment, the calibration comprises: obtaining a first conductivity or concentration measurement in a treatment fluid of the extracorporeal blood circuit running in a first direction; obtaining a second conductivity or concentration measurement in the treatment fluid running in a second direction, and calculating the access flow rate in said blood access as a function of: said first conductivity or concentration measurement and of said second conductivity or concentration measurement.

In one embodiment, the method further comprises calculating a reference fluid flow rate (Qref) at a time point when the calculated fluid flow rate (Q) corresponds to an average fluid flow rate. In this way, reference measurements are determined at instances of average fluid flow rates, suppressing effects from variability in the fluid flow rate.

In one embodiment, the method further comprises: defining an initial model; assigning the initial model to a current model; generating a parameter that correlates with the fluid flow rate; acquiring flow calibration data; investigating whether a model validity criterion is fulfilled or not by comparing parameter, calibration data with the current model, wherein in case the model validity criterion is not fulfilled then repeatedly generating a new model and assigning the current model with new model until model validity criterion is fulfilled; wherein in case the model validity criterion is fulfilled, calculating a fluid flow rate based at least partly on the temporal signal profile.

In one embodiment, the method further comprises one or more of acquiring blood pressure of the subject and comparing said blood pressure with the current model; and storing of current model and available parameters.

In one embodiment, the calculating of a fluid flow rate (Q) involves a pulse parameter P from one or more of amplitude, shape, and timing of the temporal signal profile. The pulse parameter P may also be referred to as a pulse feature.

A third aspect of the invention is a computer-readable medium comprising computer instructions which, when executed by a processor, cause the processor to perform the method of the second aspect.

A fourth aspect of the invention is a device for monitoring a fluid flow rate of a cardiovascular system of a subject, said device comprising: means for obtaining a time-dependent measurement signal from a pressure sensor in an extracorporeal blood circuit which is adapted for connection to the cardiovascular system, the pressure sensor being arranged to detect a subject pulse originating from a subject pulse generator; means for processing the measurement signal to obtain a pulse profile which is a temporal signal profile of the subject pulse; and means for calculating a fluid flow rate based at least partly on the temporal signal profile.

The calculated fluid flow rate may for instance be the cardiac output of the cardiovascular system of the subject or the access flow of a blood access in the cardiovascular system.

The signal processor of the device of the first aspect of the invention is further configured to carry out any of the steps of the methods according to the second aspect of the invention.

Embodiments of the second to fourth aspects of the invention may correspond to the above-identified embodiments of the first aspect of the invention.

Still other objectives, features, aspects and advantages of the present invention will appear from the following detailed description, from the attached claims as well as from the drawings.

### BRIEF DESCRIPTION OF THE DRAWINGS

Embodiments of the inventive concepts will now be described in more detail with reference to the accompanying schematic drawings.

FIG. 1 is a schematic view of a general fluid arrangement in which the inventive concepts may be used for monitoring a fluid flow rate in a cardiovascular system.

FIG. 2 is a partially schematic view of a forearm of a subject provided with an arterial/venous (AV) fistula.

FIG. 3 is a schematic view of a system for hemodialysis treatment including an extracorporeal blood flow circuit.

FIG. 4 (a) is a plot in the time domain of a venous pressure signal containing both pump frequency components and a heart signal, and FIG. 4(b) is a plot of the corresponding signal in the frequency domain.

FIG. 5 is a flow chart of a monitoring process according to an embodiment of the invention.

FIG. 6(a) is a plot of a pressure signal as a function of time, and FIG. 6(b) is a plot of the pressure signal after filtering.

FIG. 7 is a plot of cardiac output variations seen decreasing during a treatment.

FIG. 8 is a plot of cardiac output variations seen stable during a treatment.

FIG. 9 is a flow chart of a signal analysis process according to an embodiment of the invention.

FIG. 10 is a flow chart of a monitoring process according to an embodiment of the invention.

FIG. 11 is a block diagram of a hydraulic model of a cardiovascular system according to the present invention.

FIG. 12 is an enlargement view of the block diagram of FIG. 11.

FIG. 13 is a plot of access flow variations obtained with ultrasound measurement.

FIG. 14 is a plot showing a subject pulse parameter according to one embodiment of the invention.

FIG. 15 is a plot of fistula pressure modelled based on the heart pulse.

FIG. 16 is a plot of two phases of a heart pulse wave form illustrating one aspect of the invention.

### DETAILED DESCRIPTION OF EXAMPLE EMBODIMENTS

In the following, different embodiments for monitoring a fluid flow rate in a cardiovascular system, in particular cardiac output and access flow, will be described with reference to an exemplifying circuit for extracorporeal blood treatment.

Throughout the following description, like elements are designated by the same reference signs.

It has surprisingly been found that time-dependent pressure measurements of pulses in an extracorporeal blood circuit coupled to the cardiovascular system of a subject may reveal vital information of fluid flow rates in the cardiovascular system, such as cardiac output and access flow in for instance a fistula. The pressure pulses have origin in a pulse generator, for instance a physiological such as the heart, breathing system, or a non-physiological such as an external pulse generator inducing pulses into the cardiovascular system, such as by inducing pressure puffs with a blood pressure cuff.

In the text below, the “subject pulse” may refer to any one of the following: heart pulse or breathing pulse.

### I. General

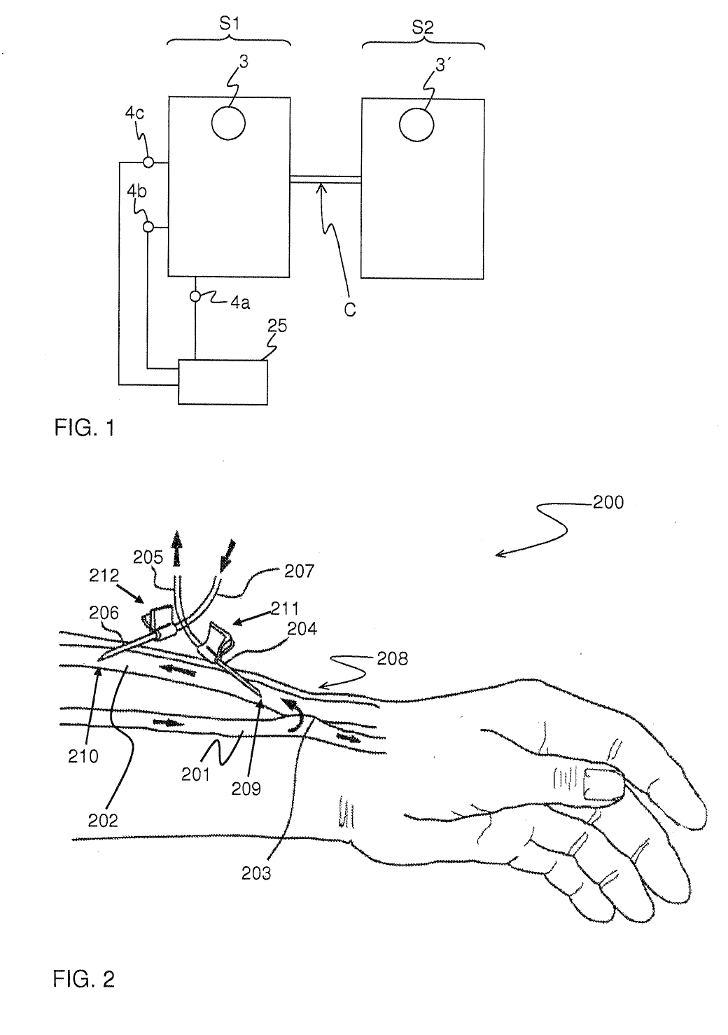

FIG. 1 illustrates a general fluid arrangement in which a fluid connection C is established between a first fluid containing system S1 and a second fluid containing system S2. The fluid connection C may or may not transfer fluid from one system to the other. A first pulse generator 3, e.g. pump, is arranged to generate a series of pressure waves in the fluid within the first system S1, and a second pulse generator 3′, e.g. a subject pulse generator such as the heart or breathing, is arranged to generate a series of pressure waves in the fluid within the second system S2. Pressure sensors 4a and 4c are arranged to measure the fluid pressure in the first system S1. As long as the fluid connection C is intact, pressure waves generated by the second pulse generator 3′ will travel from the second system S2 to the first system S1, and thus second pulses originating from the second pulse generator 3′ will be detected by the pressure sensors 4a and 4c in addition to first pulses originating from the first pulse generator 3. It is to be noted that either one of the first and second pulse generators 3, 3′ may include more than one pulse-generating device. Further, any such pulse-generating device may or may not be part of the respective fluid containing system S1, S2.

As used herein, a “pressure wave” denotes a mechanical wave in the form of a disturbance that travels or propagates through a material or substance. The pressure waves typically propagate in the fluid at a velocity of about 3-20 m/s. The pressure sensor generates measurement data that forms a pressure pulse for each pressure wave. A “pressure pulse” or “pulse” is thus a set of data samples that define a local increase or decrease (depending on implementation) in signal magnitude within a time-dependent measurement signal (“pressure signal”). The pressure pulses appear at a rate proportional to the generation rate of the pressure waves at the pulse generator. The pressure sensor may be of any type, e.g. operating by resistive, capacitive, inductive, magnetic or optical sensing, and using one or more diaphragms, bellows, Bourdon tubes, piezo-electrical components, semiconductor components, strain gauges, resonant wires, photo-plethysmography (PPG), accelerometers, bioimpedance, etc.

The fluid arrangement of FIG. 1 further includes a surveillance device 25 which is connected any one of the pressure sensors 4a-4c, as indicated in FIG. 1. Thereby, the surveillance device 25 acquires one or more measurement signals that are time-dependent to provide a real time representation of the fluid pressure in the first system S1. The surveillance device 25 monitors a vascular flow rate of the cardiovascular system of the subject, based on the principle that characteristics, such as magnitude, shape and/or phase, of the second pulses vary depending on the status of the cardiovascular system. Characteristics in the second pulses are related to the characteristics of fluid flows in the cardiovascular system, and these characteristics may be collected for subsequent analysis or comparison over time of how the various fluid flows develop and change.

The surveillance device 25 is thus configured to continuously process the time-dependent measurement signal(s) to monitor one or more vascular flows, such as the cardiac output (CO) or access flow Qₐ. Typically, the determination involves analyzing the measurement signal(s), or a pre-processed version thereof, in the time domain to calculate a value of an evaluation parameter which is indicative of the characteristics of the second pulses in the measurement signal(s). Depending on implementation, the surveillance device 25 may use digital components or analogue components, or a combination thereof, for receiving and processing the measurement signal(s).

### II. Example of an Extracorporeal Circuit

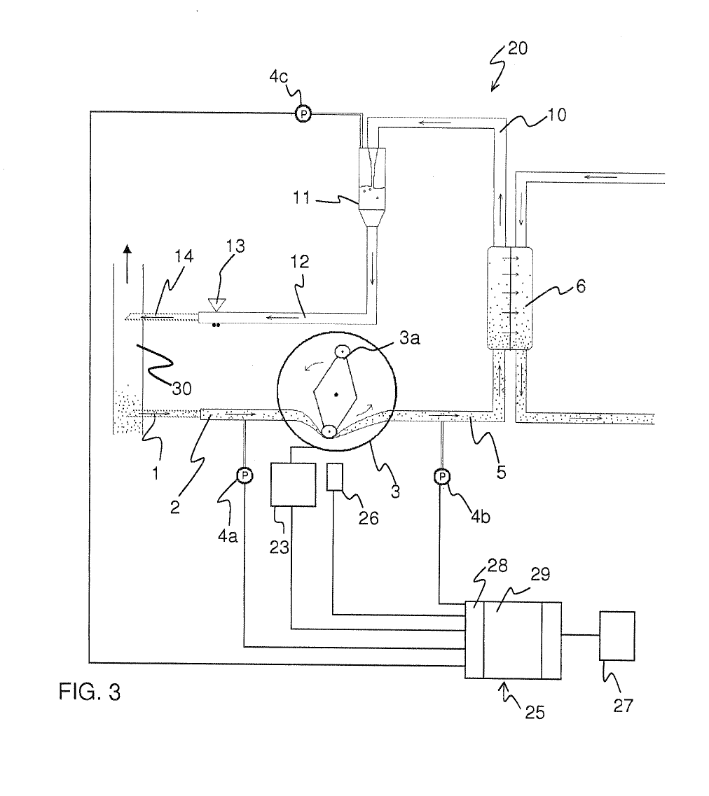

FIG. 3 exemplifies the first system S1 of FIG. 1 in the form of an extracorporeal blood flow circuit 20 of the type which is used for dialysis. The second system S2 of FIG. 1 corresponds to a subject indicated in FIG. 3 by blood vessel 30. Thus, the extracorporeal blood flow circuit 20 comprises an access device for blood extraction in the form of an arterial needle 1, and an arterial tube segment 2 which connects the arterial needle 1 to a blood pump 3 which may be of peristaltic type, as indicated in FIG. 3. Withdrawal or artery needle 1 and return or venous needle 14 are shown connected to a vessel 30 of the subject, which vessel is a part of the cardio-vascular system of the subject. At the inlet of the pump 3 there is a pressure sensor 4a, hereafter referred to as arterial sensor, which measures the pressure before the pump in the withdrawal tube segment 2. The blood pump 3 propels the blood from the fistula, through the withdrawal needle 1, via a pre-dialyser tube segment 5, to the blood-side of a dialyser 6. Many dialysis machines are additionally provided with a pressure sensor 4b that measures the pressure between the blood pump 3 and the dialyser 6. The blood is lead via a post-dialyser tube segment 10 from the blood-side of the dialyser 6 to a venous drip chamber or deaeration chamber 11 and from there back to the subject via the return tube segment 12 and return needle 14. A pressure sensor 4c, hereafter referred to as venous sensor, is provided to measure the pressure on the venous side of the dialyser 6. In the illustrated example, the pressure sensor 4c measures the pressure in the venous drip chamber. Both the withdrawal needle 1 and the return needle 14 are connected to the subject by means of the vascular access.

As discussed by way of introduction, it may be vital to monitor the fluid flows in a cardiovascular system with respect to variations, levels and/or changes. In many dialysis monitors, one or more of said pressure detectors 4a-4c are not present. However, there will be at least one venous pressure sensor. The following description is focused on monitoring various vascular flows, with specific examples relating to the cardiac output (CO) and access flow Qₐ based on a measurement signal from one or more of the pressure sensors.

Further in FIG. 3, a control unit 23 is provided, i.a., to control the blood flow in the circuit 20 by controlling the revolution speed of the blood pump 3. The extracorporeal blood flow circuit 20 and the control unit 23 may form part of an apparatus for extracorporeal blood treatment, such as a dialysis machine. Although not shown or discussed further it is to be understood that such an apparatus performs many other functions, e.g. controlling the flow of dialysis fluid, controlling the temperature and composition of the dialysis fluid, etc.

Also in FIG. 3, a surveillance device 25 is configured to monitor various vascular flows, with specific examples relating to the cardiac output (CO) and access flow Qₐ, by analysing pressure response for instance originating from the subject's heart in a blood pressure pulse signal. The surveillance device 25 is connected to receive a measurement signal from any of the pressure sensors 4a-4c. The device 25 may also be connected any additional pressure sensors included in the extracorporeal blood flow circuit 20. As indicated in FIG. 3, the device 25 may also be connected to the control unit 23. Alternatively or additionally, the device 25 may be connected to a pump sensor 26, such as a rotary encoder (e.g. conductive, optical or magnetic) or the like, for indicating the frequency and phase of the blood pump 3. The surveillance device 25 is tethered or wirelessly connected to a local or remote device 27 for continuously presenting updated values of one or more of the determined vascular flows on a screen, storing on a memory and/or generating an audible/visual/tactile alarm or warning signal in case of one or more vascular flows of the cardiovascular system falls below acceptable levels. For instance, occurrence of recirculation may be detected. In order to avoid recirculation, i.e. treated blood return re-enters the withdrawal line, the average or momentary access flow rate Qa should be greater than the sum of blood flow rate Qb withdrawn from the access and a component relating to variability in the access flow. The surveillance device 25 and/or the remote device 27 may alternatively be incorporated as part of a dialysis monitor.

Additionally, in FIG. 3, the surveillance device 25 comprises a data acquisition part 28 for pre-processing the incoming signal(s), e.g. including an A/D converter with a required minimum sampling rate and resolution, one or more signal amplifiers, one or more filters to remove undesired components of the incoming signal(s), such as offset, high frequency noise and supply voltage disturbances.

In the examples given herein, the data acquisition part 28 comprises a DAQ card USB-6210 from National Instruments with a sampling rate of 1 kHz and resolution of 16 bits, an operation amplifying circuit AD620 from Analogue Devices, a high-pass filter with a cut-off frequency of 0.03 Hz (i.a., for removal of signal offset) together with a low-pass filter with a cut-off frequency of 402 Hz (i.a., for removal of high frequency noise). To obtain a short convergence time, a low-order filter is used for the high-pass filter. Furthermore, the data acquisition part 28 may include an additional fixed band-pass filter with upper and lower cut-off frequencies of 0.5 Hz and 2.7 Hz, respectively, which corresponds to heart pulse rates between 30 and 160 beats per minute. This filter may be used to suppress disturbances outside the frequency interval of interest. Corresponding filters may be applied to extract pressure pulses originating from breathing or other physiological signals, which may be used separately or in combination with the heart pulse rates to monitor the fluid flows in the cardiovascular system.

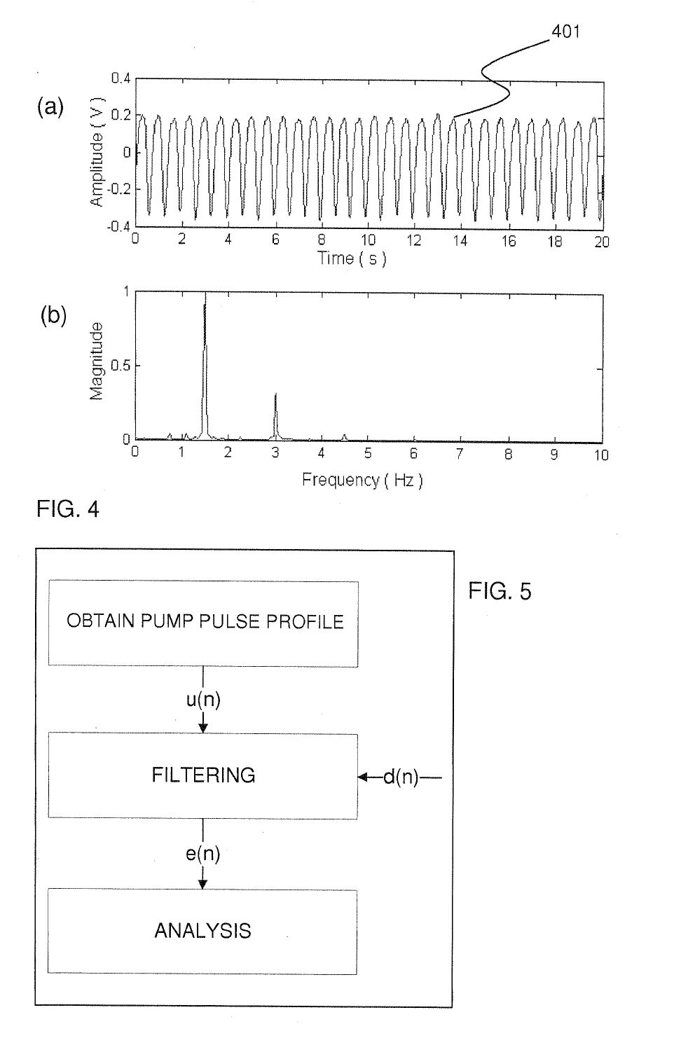

After the pre-processing in the data acquisition part 28, the pre-processed pressure signal is provided as input to a main data processing part 29, which executes the inventive data processing. FIG. 4(a) shows an example of such a pre-processed pressure signal 401 in the time domain, and FIG. 4(b) shows the corresponding power spectrum, i.e. the pre-processed pressure signal in the frequency domain. The power spectrum reveals that the detected pressure signal contains a number of different frequency components emanating from the blood pump 3. In the illustrated example, there is a frequency component at the base frequency (f₀) of the blood pump (at 1.5 Hz in this example), as well as its harmonics 2f₀, 3f₀ and 4f₀. The base frequency, also denoted pump frequency in the following, is the frequency of the pump strokes that generate pressure waves in the extracorporeal circuit 20. For example, in a peristaltic pump of the type shown in FIG. 3, two pump strokes are generated for each full revolution of the rotor 3a. FIG. 4(b) also indicates the presence of a frequency component at half the pump frequency (0.5f₀) and harmonics thereof, in this example at least f₀, 1.5f₀, 2f₀ and 2.5f₀. FIG. 4(b) also shows a heart signal (at 1.1 Hz) which in this example is approximately 40 times weaker than the blood pump signal at the base frequency f₀.

Typically, the surveillance device 25 is configured to continuously process the time-dependent pressure signal(s) to isolate subject pulses originating from for instance a patient's heart. This processing is schematically depicted in the flow chart of FIG. 5. The illustrated processing involves a step 501 of obtaining a pump pulse profile u(n) which is a predicted temporal signal profile of the pump pulse(s), and a step 502 of filtering the pressure signal d(n), or a pre-processed version thereof, in the time-domain, using the pump pulse profile u(n), to essentially eliminate or cancel the pump pulse(s) while retaining the subject pulse(s) contained in d(n). In the context of the present disclosure, n indicates a sample number and is thus equivalent to a (relative) time point in a time-dependent signal. In step 503, the resulting filtered signal e(n) is then analysed for the purpose of monitoring the aforesaid parameter.

The pump pulse profile is a shape template or standard signal profile, typically given as a time-sequence of data values, which reflects the shape of the first pulse in the time domain. The pump pulse profile is also denoted “predicted signal profile” in the following description.

By “essentially eliminating” is meant that the pump pulse(s) is(are) removed from the pressure signal to such an extent that the subject pulse(s) can be detected and analysed for the purpose of monitoring the aforesaid parameter.

By filtering the pressure signal in the time-domain, using the pump pulse profile, it is possible to essentially eliminate the pump pulses and still retain the subject pulses, even if the pump and subject pulses overlap or nearly overlap in the frequency domain. Such a frequency overlap is not unlikely, e.g. if one or both of the pump and subject pulses is made up of a combination of frequencies or frequency ranges.

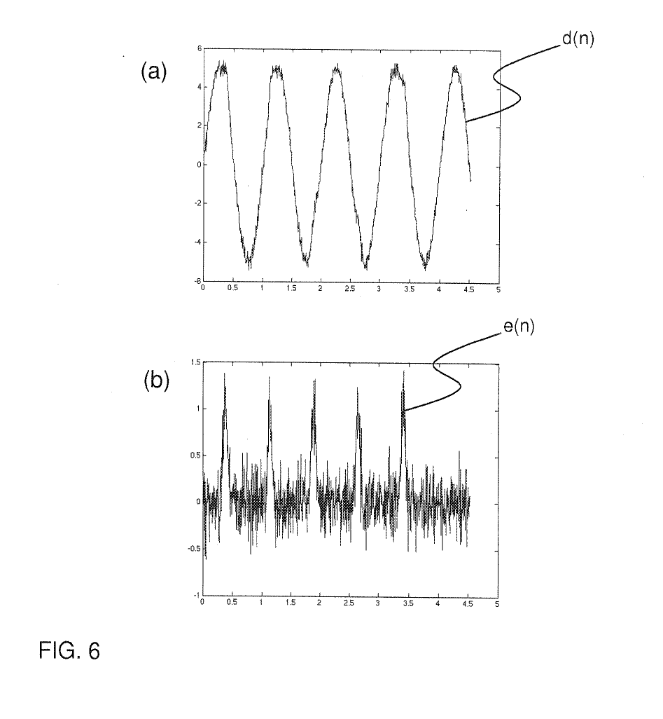

The effectiveness of the inventive filtering is exemplified in FIG. 6, in which FIG. 6(a) shows an example of a time-dependent pressure signal d(n) containing pump and subject pulses with a relative magnitude of 10:1. The pump and subject pulses have a frequency of 1 Hz and 1.33 Hz, respectively. Due to the difference in magnitude, the pressure signal is dominated by the pump pulses. FIG. 6(b) shows the time-dependent filtered signal e(n) that is obtained after applying the inventive filtering technique to the pressure signal d(n). The filtered signal e(n) is made up of subject pulses and noise.

The main data processing part 29 executes the aforesaid steps 501-503 of FIG. 5. In step 502, the main data processing part 29 operates to filter the pre-processed pressure signal in the time domain, and outputs a filtered signal or monitoring signal (e(n) in FIG. 5) in which the signal components of the blood pump 3 have been removed. The monitoring signal still contains any signal components that originate from the subject (cf. FIG. 6(b)), such as pressure pulses caused by the beating of the patient's heart, breathing or other physiological signals, the latter two which, in a case where the heart is the primary source for analysis, also may be removed to reduce noise from the heart pulses to facilitate the analysis. In another situation breathing is the primary source for analysis, and the heart and other physiological signals may be removed. The signal components may additionally involve artificial origin accounting for modulation from unwanted noise, for instance by a separate, external pressure inducing component, such as integrated in a blood pressure cuff, or the blood pressure cuff itself with pressure waves induced by puffing air into the cuff. A further source of unwanted noise may origin from vibrations, and thus pressure waves, resulting from coughing, sneezing, vomiting and seizures.

Hence, depending on implementation, the surveillance device 25 may be configured to apply filtering to the monitoring signal to isolate signal components originating from a single cyclic phenomenon in the subject, such as the heart pulse or breathing. Alternatively, such signal component filtering is done during the pre-processing of the pressure signal (by the data acquisition part 28). The signal component filtering may be done in the frequency domain, e.g. by applying a cut-off or band pass filter, since the signal components of the different cyclic phenomena in the patient are typically separated in the frequency domain. Generally, the heart frequency is about 0.5-4 Hz, the breathing frequency is about 0.15-0.4 Hz, the frequency of the autonomous system for regulation of blood pressure is about 0.04-0.14 Hz, the frequency of the autonomous system for regulation of body temperature is about 0.04 Hz.

The surveillance device 25 may be configured to collect and store data on the evolution of the amplitude, phase, shape, etc, e.g. for subsequent analysis in connection with monitoring the condition of the subject.

The surveillance device 25 may be configured to monitor the fluid flows in the cardiovascular system of the subject, in particular cardiac output (CO) and access flow Qₐ. This may be done by monitoring characteristics of a signal component originating from, e.g., the patient's heart or breathing system in the monitoring signal or the monitoring signal itself where the composite signal is analysed.

The extracorporeal circuit 20 may have the option to operate in a hemodiafiltration mode (HDF mode), in which the control unit 23 activates a second pumping device (HDF pump, not shown) to supply an infusion solution into the blood line upstream and/or downstream of the dialyser 6, e.g. into one or more of tube segments 2, 5, 10 or 12.

The obtaining of the predicted signal profile of pulses originating from a pump is described i more detail in the section “Obtaining the predicted signal profile of first pulses” of Appendix A.

In addition, an external signal source, not shown, such as a photoplethysmograph (PPG) or an electrocardiograph (ECG) signal may be used as a timing reference to the pressure based signal originating from the actuation of the heart.

### III. Example of a Fistula

For a better understanding of the inventive concept, for instance with regards to measuring flows in the access system, which is related to a fistula, the anatomy and interconnection with a fistula will be described in the following with reference to FIG. 2.

FIG. 2 discloses a forearm 200 of a subject. The forearm 200 comprises an artery 201, in this case the radial artery, and a vein 202, in this case the cephalic vein. The blood flow in a fistula is referred to as access flow Qa, and in FIG. 2 the blood flow in the artery (201) and vein (202) are indicated with arrows. Openings are surgically created in the artery 201 and the vein 202 and the anastomosis are connected to form an anastomosis 203, in which the arterial blood flow is short-circuited to the vein. Such a configuration with the anastomosis 203 and nearby sections of the artery 201 and vein 202 are commonly referred to as a fistula 208. Due to the fistula, the blood flow through the artery and vein is increased and the vein forms a thickened area downstream of the connecting openings. When the fistula has matured a few months after surgery, the vein is thicker and may be punctured repeatedly.

An arterial or withdrawal device 211 in the form of a needle 204, to which is connected a piece of arterial or withdrawal tube 205, is placed in an upstream position 209 in the fistula, in the enlarged vein close to the connected openings and a venous or return device 212 also in the form of a needle 206, to which is connected a piece of venous or return tube 207, is placed in a position downstream 210 of the arterial or withdrawal needle 204, normally at least five centimetres downstream thereof. The withdrawal 205 and return 207 tubes are connected to an extracorporeal circuit (not shown) such as described in FIG. 3. In use, the withdrawal tube 205 may transport blood from the artery 201 via the arterial or withdrawal needle 204 to an inlet of the extracorporeal circuit, and the return tube 207 then returns the treated blood from an outlet of the extracorporeal circuit to the vein 202 via the venous or return needle 206. Arrows at the ends of the blood lines (205, 207) indicate the direction of blood flow in a normal configuration.

The vascular access may also be an arterio-venous graft, Scribner-shunt, one or more catheters, a double lumen catheter or other similar arrangements. For the purpose of the following discussion, the blood vessel access is assumed to be a fistula. The withdrawal and return needles may also be catheters. The withdrawal and return devices generally comprises a needle or catheter, a tubing and a connector (not shown) connecting the tubing to the needle or catheter.

The needles 204 and 206 are connected to a tube system, shown in FIG. 3, forming an extracorporeal blood flow circuit 20 of the type which is used for dialysis.

### IV. Modelling of a Cardiovascular System

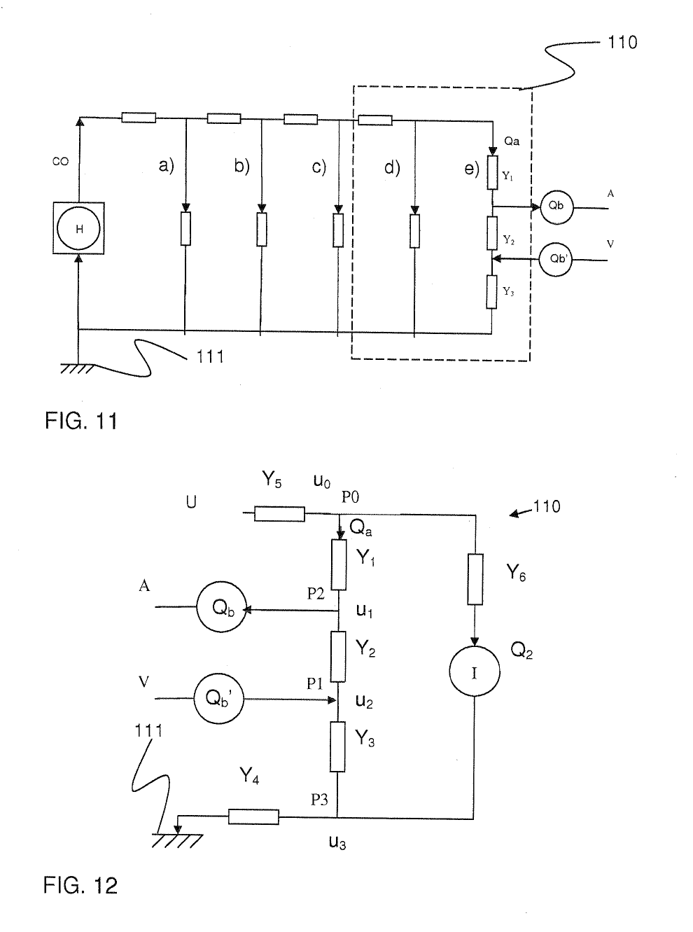

For a better understanding of the inventive concept, a hydraulic model of the cardio-vascular system will be described in the following with reference to FIG. 11.

The access flow Qa in a fistula is related to other flows in the cardiovascular system, such as Cardiac Output (CO), which is the total blood flow from the heart.

Generally, fluid flow rate (Q) and flow admittance (Y) are measures which may be used to describe the function and condition of a cardiovascular system. Particularly, the fluid flow rate (Q) and flow admittance (Y) may provide vital information of the heart function and condition of a fistula, and how it changes over time, for instance during a treatment or between treatments. Fluid flow rate (Q) may also be used to determine where, for instance in a fistula, a problem may reside. The inverse of admittance (Y) is impedance (Z), both which may be referred to in the following.

FIG. 11 shows a schematic diagram of a hydraulic model of the cardio-vascular system connected to an extra-corporeal circuit. The blood flow at any location in the cardio-vascular system is related both to the activity of the heart (H), e.g. referred to as the Cardiac Output (CO), and to the distribution of vascular flow impedance (Z) in the vascular system. The vessels actively change diameter under the influence of physiology, for instance by temperature regulation, or therapy, vasoconstrictors decrease vessel diameter and increase impedance, while vasodilators increase vessel diameter and decrease impedance due to heart activity regulation by the autonomous system. Hence, an increase in impedance decreases the Cardiac Output (CO). Due to the cardiac output (CO) and admittance (Y) in the cardio-vascular system, the mean arterial pressure (MAP), is generated. The Cardiac Output (CO) is distributed into the organs and limbs through a complex network of blood vessels, illustrated with branches a)-e) in FIG. 11. Each branch is associated with a certain flow admittance.

As long as the impedance properties of the vascular system are fairly stable over an investigated period of time, the relative flow variation at a certain point may be approximated by the relative flow variation at any other points of the system according to changes in the activity of the heart. Although not necessarily being linear, the relationship between a change of flow in one branch to the corresponding value of another branch is generally largely governed by a certain mathematical model of the cardiovascular system. Therefore, access flow variations may for instance be determined from Cardiac Output (CO) variations and vice versa. Variations of blood flow to e.g. an organ or a limb may be estimated based on determinations of blood flow variations in another location. Absolute flows however, need to be determined via calibration with respect to reference measurements or hydraulic models of the vascular system. Also the model may be subjected to calibration, e.g. by any of the previously mentioned reference methods. At least one point is needed for the calibration, but additional points would allow determination of linear and non-linear relationships that may exist between the measured pressure and the blood flow. For instance, provided that aforesaid relationship is linear, two reference measurements would suffice to provide calibrated fluid flow rate values of the method according to the invention.

A blood access is exemplary modelled with three admittance symbols to the right in the figure within the dashed rectangle 110, which is enlarged and explained in detail with reference to FIG. 12. Here, the access flow is represented by Qa, Qb is blood flow withdrawn from the blood access and Qb′ is the blood flow returned to the blood access during dialysis. “A” and “V” represents the “arterial” and “venous” side of the extra-corporeal circuit. Also shown in the model are body admittance boxes Y₁, Y₂ and Y₃ related to the flow and pressure characteristics of the vascular branch comprising the fistula. The earthing symbol 111 indicates that the absolute pressure is low at the venous return to the heart, i.e. close to zero relative the atmospheric pressure according to known principles.

FIG. 12 shows a close-up 110 of a blood access branch of the cardio-vascular system of FIG. 11, the branch also comprising a fistula here represented by the three flow admittances Y₁, Y₂ and Y₃. Other fistula configurations may be modelled according to the same principles.

The fistula configuration has been translated into a hydraulic model as is shown in FIG. 12, where the blood flow in the access devices is indicated with arrows. Table 1 below lists the modelling parameter definitions that are used in FIG. 11 and FIG. 12 and related to the analysis. Additionally, P0 denotes a position in the artery close to the anastomosis. P1 denotes an arterial access point in fistula, P2 denotes a venous access point in fistula, and P3 denotes a common venous return point for the blood flow in the artery, i.e. Qₐ+Q₂.

Since the model takes dynamic as well as static effects into account, it may be applied to pressure levels as well as pressure variations, hence including effects due to propagation of pressure pulses and admittance in the system. In dynamic modelling, frequency dependence arises due to damping in the system. Generally, the pressure pulse propagation speed may be modulated by e.g. blood pressure.

TABLE 1
Modelling parameter definitions

UFR: ultrafiltration rate
Q_{b}: blood flow of dialysis machine
Z = impedance
Y = 1/Z, i.e. admittance is the inverse of impedance
Y₁: flow admittance between anastomosis (P0) and arterial access point
(P1), ‘pre-fistula admittance’
Y₂: flow admittance between arterial (P1) and venous (P2) access points,
‘intra-fistula admittance’
Y₃: flow admittance between venous (P2) access point and common
venous return vessel (P3), ‘post-fistula admittance’
Y₄: flow admittance in vein between common venous return vessel (P3)
and venous return to heart (111)
Y₅: flow admittance in artery entering the fistula
Y₆: flow admittance of vessels supplying tissue in the arm/hand access
u₀: arterial blood pressure at the anastomosis (P0), e.g. reflected
by mean arterial pressure (MAP)
Normal position of access needles:
u1: blood pressure at the arterial access point 1 (P1)
u2: blood pressure at the venous access point 2 (P2)
u3: blood pressure at common venous return vessel (P3)
Q_{b}: blood flow pumped out of the fistula
Q_{b}′: blood flow pumped back to the fistula: Q_{b}-UFR
Q₂: arterial blood flow required by the tissues downstream of the fistula,
Qₐ: blood access flow entering the fistula

In the model, it has been assumed that the blood pressure of the artery e.g. reflected by mean arterial pressure MAP is controlled by autonomous system to a constant value (u₀), the demand of nutrition and oxygen of tissue, the arterial blood flow in the branch of the fistula is constant (Q₂) and that all flow admittances (Y₁-Y₄) do not vary. Both Y₅ and Y₆ are omitted in the modelling, since they are assumed to be very large.

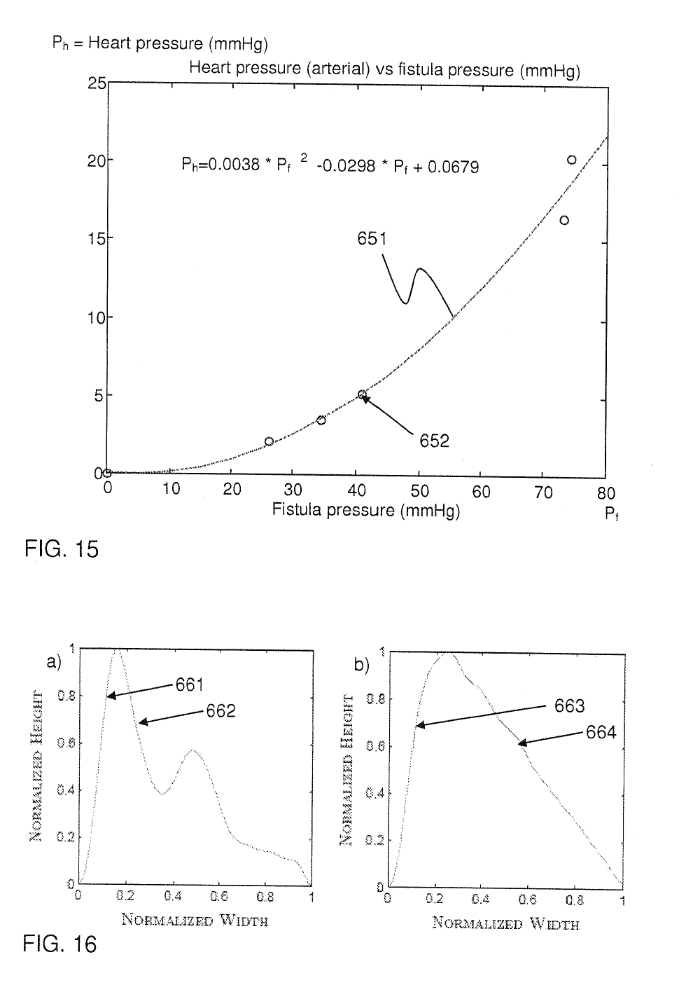

FIG. 15 shows how the amplitudes of the heart pressure signals at A or V in FIG. 12 depend on the fistula pressure, e.g. it is possible to determine a relationship from the heart amplitude in A or V with the fistula pressure for the corresponding access point in the fistula.

V. Cardiovascular Fluid Flow Rate from Pressure Signal

In the present invention, methods for analysing the pulse pressure are used to determine a waveform and using this information to calculate cardio-vascular measures such as related to the cardiac performance and blood access branches of the cardiovascular system. The inventive concept is based upon the relation that pressure variations in an extracorporeal circuit in fluid connection with a cardiovascular system may be used to derive pressure and flow variations in the cardiovascular system. Thus, variations in Cardiac Output (CO) and access flow are both related to variations in the heart signal measured by the present invention. Hence, by monitoring pressure variations in the extracorporeal circuit and relating these variations with relevant cardiovascular relationships, cardiovascular fluid flow rates may be determined.

The present invention generally relates to variations in fluid flow rates in a cardiovascular system causing variations in fistula pressure which in turn cause changes in the heart pulses obtained from an extracorporeal pressure sensor, which changes may be quantified by a parameter P involving for instance amplitude, shape, phase and/or frequency or timing.

Monitoring of the cardiovascular fluid flow rates may provide vital information of a subject, for instance cardiac function and/or blood access function, which will be described in more detail in separate sections below.

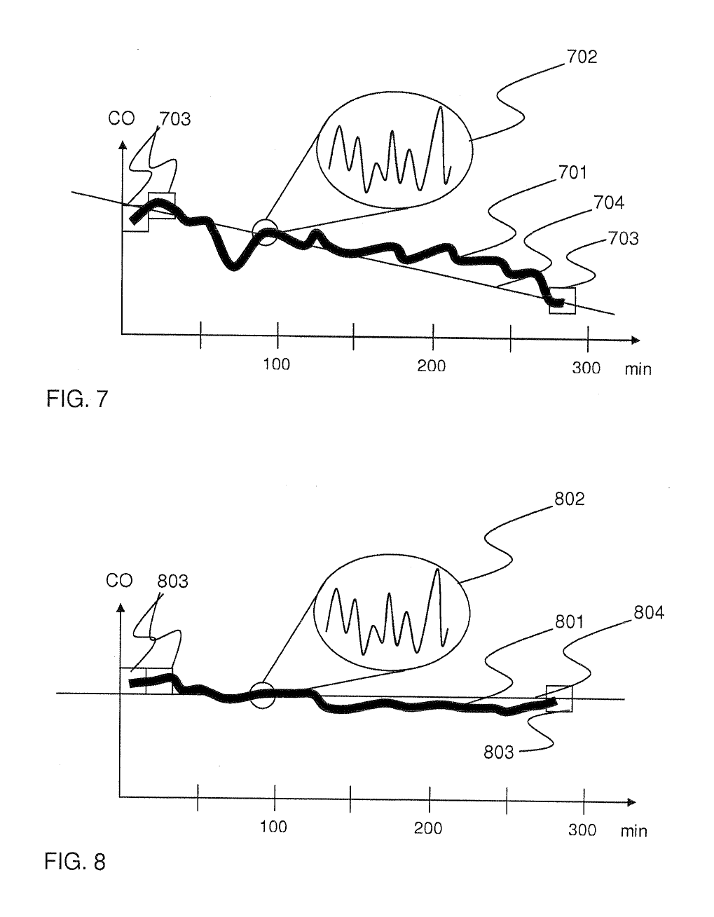

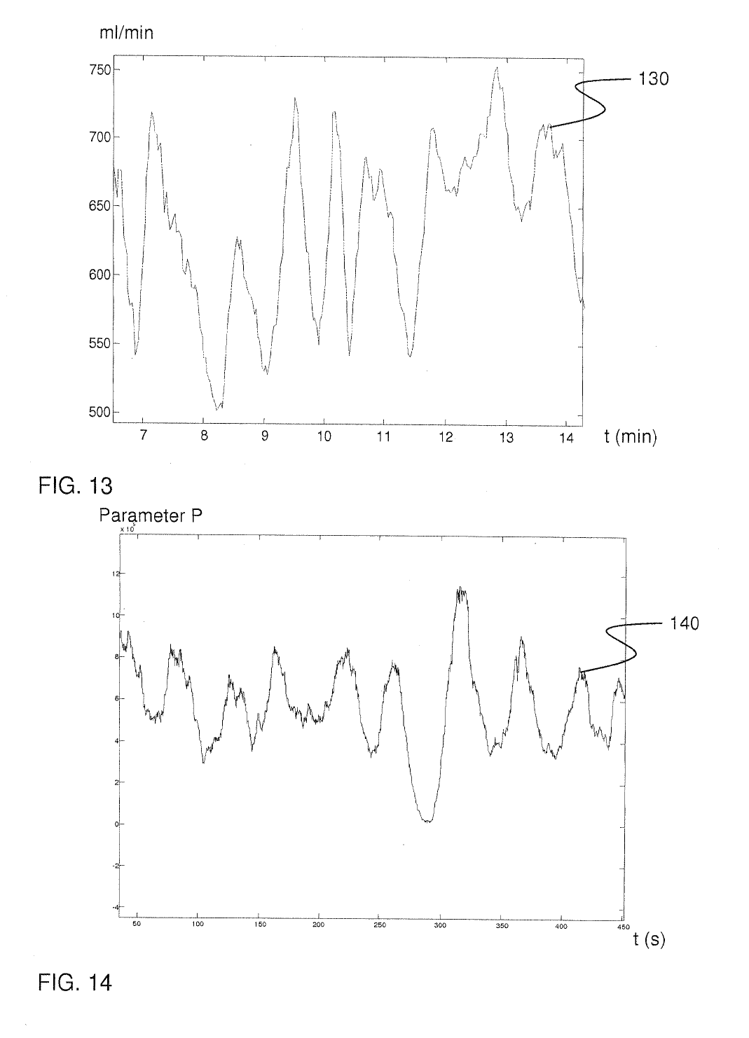

FIG. 7 and FIG. 8 illustrate monitoring of cardiac output variations during two different treatments having durations of approximately five hours each. Similar variations would be seen also in for instance access flow measurements. Similar recordings may be generated for a number of subsequent treatments to get an overall clinical picture. FIG. 7 illustrates measurements from a subject where cardiac output 701 is decreasing during a dialysis treatment, as indicated by trend line 704. FIG. 8 illustrates measurements from a patient where cardiac output 801 is relatively stable, as indicated by trend line 804. In the present invention, it has surprisingly been found that periodic variations can been seen on a smaller time scale. The close-ups 702 and 802 in the figures illustrate segments where such periodic variations in the range of approximately 50 seconds and representing a relative flow variation of about 10-30% may be seen. These periodic variations are explained in more detail in connection with FIG. 13 and FIG. 14 below. The squares 703 and 803 illustrate segments of time periods wherein calibrations have been performed with a reference method during the treatment. Calibration may of course be performed anytime during the treatment, and may be used between treatments, for instance by storing the values in a memory, but is preferably at least performed at the beginning and/or end of the treatment. Two initial calibrations are also shown which may provide improved accuracy, for instance to determine and/or improving e.g. a linear relationship between relative measurements and absolute values.

The analysis of pressure variations in the pressure signal may comprise various measures, such as amplitude, shape and phase. For instance, the amplitude of peak maxima in the pressure signal may be proportional to a cardio-vascular flow. Additionally, due to damping and delay of the frequency components of the heart pulses affecting their shape and/or phase, predetermined profiles representing a particular cardio-vascular flow may be mapped against a measured pressure pulse profile.

By way of example, plots representing access flow rate variations (on the Y-axis) obtained with a prior art method, and with the present invention will now be illustrated in FIG. 13 and FIG. 14, respectively. The horizontal axis represents different time periods of approximately six to 8 minutes each. In both figures, variations are present, the variations having a period in the range of 40 to 60 seconds.

FIG. 13 and FIG. 14 show variations in access flow rate in a relatively short time-scale as indicated with the close-ups 702 and 802 in FIGS. 7 and 8 respectively. FIG. 13 and FIG. 14 do not necessarily correspond to the same time period of the same treatment. FIG. 13 shows actual access flow variations 130 obtained with an ultrasound (Doppler) measurement. The x-axis represents time in minutes and the y-axis represents flow rate in ml/min. Short-term variations in the access flow rate of approximately a minute in duration are clearly visible.

FIG. 14 shows a cross-correlation measure 140 between heart signals obtained from the arterial and venous pressure sensors according to one aspect of the present invention. Alternatively, either of the heart signals from the arterial or venous pressure sensors may be used, although a cross-correlation between the two may suppress unwanted components. The cross-correlation measure is shown before calibration and indicates the relative variation. The x-axis represents time in seconds and the y-axis represents access flow rate of an arbitrary unit calculated from the amplitude of the heart pulses. Other parameters that may be used in the calculation include shape, frequency, phase and variability. Also, amplitude variations 140 of similar periodicity are clearly visible. Hence, FIG. 14 illustrates that access flow rate variations, similar to those in FIG. 13, may be monitored by pressure sensors in the extra-corporeal circuit. These variations may be used to better determine the absolute value of both cardiac output and access flow when the corresponding reference methods are used for calibration.

An advantage with the present invention is that it may be built into for instance a dialysis monitor and that it does not require additional instruments and sensors, particularly when the monitor is further configured to measure reference values of for instance access flow Qa and Cardiac Output (CO). Especially, it provides a continuous “beat-to-beat” measure. Actually, even different measurements within a pulse is possible with the present invention.

There are of course other techniques for calculating the evaluation parameter value P, including other types of time domain analyses, as well as different types of frequency domain analyses.

Other factors, such as the medical history of the patient, e.g. heart status, blood pressure and heart rate may also be utilized for improving the vascular flow determination.

Pressure data extracted from the measurement signal may be represented as a temporal pulse profile in the time domain. The temporal pulse profile may be transformed into a frequency spectrum and/or a phase spectrum as another pressure data representation. From the pressure data, a parameter value may be calculated. For example, the parameter value may be related to the amplitude, shape, frequency and/or phase, or timing of the pressure pulse.

The performance of the reference methods may be improved since the measurement period of these methods are shorter in time than the period of the variations presented in FIG. 13 and FIG. 14. Thus, when an absolute value is obtained by the reference methods it will be affected by the magnitude of the inherent flow variations and it will also be dependent on when in time the measurement is performed. The variation in FIG. 14 may be used in order to improve performance of the calibration methods by taking into account e.g. the time of injection, duration of injection, and circulation times in the cardio-vascular system.

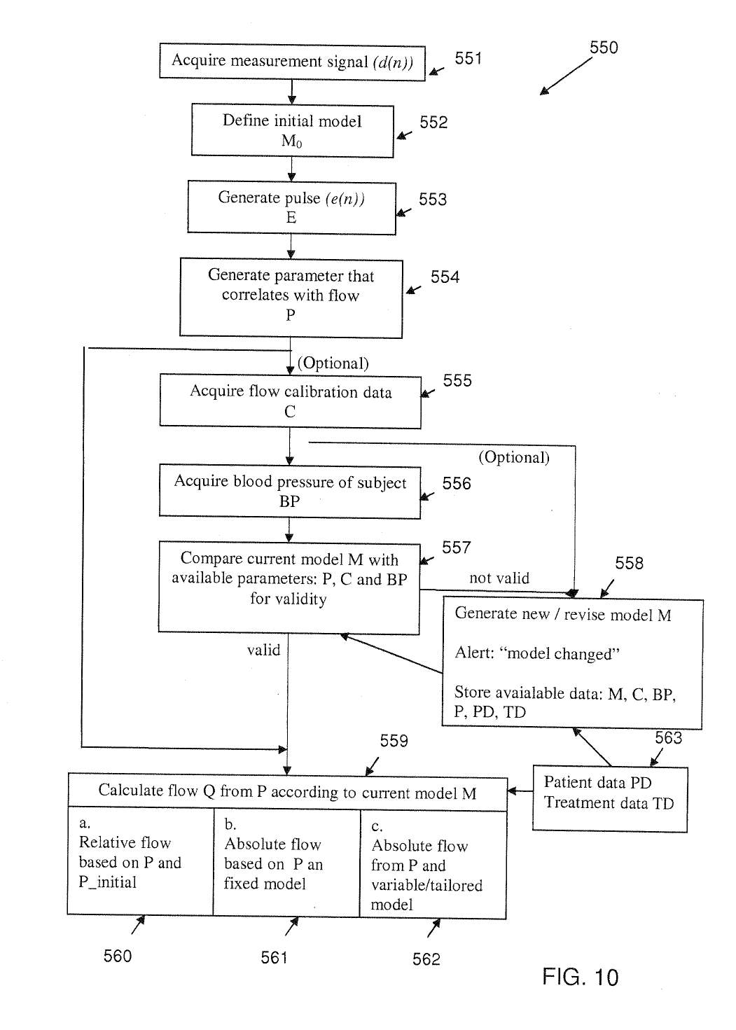

With the pulse wave analysis of the present invention, it is now possible to improve the absolute measurements. It is even possible to improve the reference measurement methods. The high resolution in the pulse wave analysis reveals more details of the relative variations of access flow and cardiac output than previously perceived. Further details are given below in connection with calibration step 555 of FIG. 10.

According to one way of improving absolute measurements of average fluid flow rate, a reference fluid flow rate (Qref) is calculated at a time point when the calculated relative fluid flow rate (Q) corresponds to an average fluid flow rate. In this way, reference measurements are determined at instances of average fluid flow rates, suppressing effects from variability in the fluid flow rate. Thus, it is not necessary to calculate a relationship between reference values relative measurement values.

Additionally, provided that the properties of the vascular system remain constant over time, i.e. no stenosis formation, calibration of the cardiac output measurement according to the present invention may remain valid and be used for monitoring of long-term changes of the cardiac output.

The present invention discloses a method for measuring the access flow rate, cardiac output and other cardiovascular flow rates almost continuously, dependent essentially only on update frequency and subject pulse rate. The variability associated with reference methods due to cardiovascular flow rate variation may thus be reduced by using data for the corresponding time period together with an estimation of the variability obtained by the present invention.

### VI. Monitoring Cardiac Output

Cardiac output is the quantity of blood pumped each minute by the heart into the aorta, i.e. the total blood flow in the circulation of a subject. During a dialysis treatment, the cardiac output is often referred to as the intra-dialytic cardiac output.

According to one embodiment of the present invention, variations in Cardiac Output (CO) are obtained by tracking the amplitude in the heart pulses obtained from an extracorporeal pressure sensor, based on the relation that a variation in Cardiac Output (CO) causes a variation in the amplitude of the heart pulses obtained from an extracorporeal pressure sensor. As an alternative to tracking the amplitude, also shape, phase and frequency or the integral of values within a time window may be used. The time window may contain one or several heart pulses as well as fractions of heart pulses. Alternatively, the sum of absolute or squared values within a time window may also be used, i.e. an energy equivalent.

Calibration of the relative measure to get an absolute measure of intra-dialytic cardiac output (CO) may be obtained by using an indicator dilution method as is described in international patent application published as WO 2005/049113, in which concentrated saline is used as the indicator and the conductivity in the spent dialysate is measured. A known amount of concentrated NaCl, e.g. 2 ml, in the form of a short duration bolus is given in the venous return line of the extracorporeal circuit. On its way back to the subject's heart, the bolus will meet returning blood from the rest of the body, pass the lungs and go back to the heart. It will then be pumped out to the body again with a flow rate determined by the Cardiac Output. As the bolus spreads through the cardio-vascular system and out in the extracorporeal circuit, a fraction of the original bolus of NaCl will be measured. The fraction reaching the spent dialysate will be clearance (K), which is the flow of blood passing the dialyzer that is completely cleared from a waste substance such as urea, divided by Cardiac Output, i.e. (K/CO). This fraction is measured as a conductivity increase in the spent dialysate, and the area under the curve is measured. The conductivity area is converted to a NaCl concentration area using the known specific conductivity of NaCl. Multiplication with the dialysate flow rate then gives the total amount of NaCl in the spent dialysate. The fraction of this measured outlet pulse in relation to the original bolus will then equal K/CO, so that if clearance (K) is measured, the Cardiac Output (CO) may be calculated.

Alternatively, measurements of the bolus dilution may also be performed with ultra-sound detection, relying on measurement of transient changes in ultrasound velocity induced by an indicator e.g. saline that has been added to the blood. A known amount of an indicator substance e.g. saline, is injected into the blood stream and its dispersion cause changes in the ultrasound velocity which is related to the concentration of the indicator.

Additionally, with embodiments of the present invention it is possible to improve the above mentioned reference method and other reference methods. A better estimate of an average absolute value from a reference measurement may be accomplished by accounting for the variations in the access Cardiac Output (CO) measured by continuous measurements according to embodiments of the present invention. For instance, by continuously tracking a relevant parameter of the blood pressure pulses, it is possible from the observed variations to determine when a calibration method should begin for instance a bolus injection and when to start detection of a response of the bolus injection. If two or more absolute values are obtained from a calibration method, preferably close in time, at different time instances in the variation curves in FIG. 14, then these absolute values may be combined together with the time instances in the variation curves in order to obtain a better estimate of the absolute average value. For example, if two values are obtained at the time instances of the maximum and minimum values of the variation curves, then a better estimate of the average absolute value would be the mean of these two values. Alternatively, the middle point in time between a max and min may be used for reference measurement to get a good estimate of the average flow.

### VII. Monitoring Access Flow

The branch of the cardio-vascular system that passes a blood access is associated with an access flow (Qa), which may be determined with the present invention.

According to embodiments of the present invention, variations in the access flow are obtained by tracking the amplitude, shape, frequency and/or phase, or timing information of for instance the heart pulses obtained from an extracorporeal pressure sensor, based on the relation that a variation in access flow causes a variation in for instance the amplitude of the heart pulses obtained from an extracorporeal pressure sensor. Instead of the amplitude, any of the other characteristics may equally well be used. This is illustrated in FIG. 14. As an alternative to tracking the amplitude, shape, phase and frequency or the integral of values of the pulse profile within a time window may also be used. The time window may contain one or several heart pulses as well as fractions of heart pulses. Alternatively, the sum of absolute or squared values within a time window may also be used.

Calibration of the relative measure to get an absolute measure may be obtained with for instance conductivity-based methods, such as referred to as a Cond-Step method, as is described in international patent application published as WO 03/066135, in which a difference in conductivity between two measurements is measured, with the blood flow being reversed between the two measurements, i.e. the connectors of the venous and arterial needle are interchanged causing recirculation of the withdrawn blood flow Qb. This will decrease the treatment efficiency to a certain degree, from which the access flow rate can be calculated. The change in treatment efficiency is measured as a change in the outlet conductivity. The treatment efficiency is proportional to the distance between inlet and outlet conductivities of the dialysis fluid. The access flow rate is inversely proportional to the change in this distance.

When performing this measurement it is necessary to ensure a difference between the inlet and outlet conductivities that is large enough. If there is no difference there will be no conductivity change when the needles are switched, and no calculations can be performed. Therefore any measurement with the Cond-Step method starts with a step in the inlet conductivity. This step is used also to obtain a clearance value, which is also needed in the access flow calculation.

The Cond-Step reference method provides up to approximately four measurement values per hour during a treatment. Each value represents access flow rate during period of approximately ten to twenty seconds and due to variations of the access flow rate with a cycle of about a minute during treatment, this and other reference methods provide only limited approximations of average access flow rate.

Other reference methods involve urea measurements such as described in WO 00/24440 or dilution measurements such as a method of Transonic where magnitude of access recirculation is determined from dilution measured with ultrasound as the ratio between the amount of indicator arriving in the arterial line during its first transit and the injected amount of indicator. Access flow Qa is related to the forced recirculation R of the reversed access position, blood flow Qb and the ultrafiltration flow rate UFR according to: Qa=(1−R)*(Qb−UFR)/R.

Similarly to the Cardiac Output measurements, embodiments of the present invention may be also used to improve the above mentioned and other reference methods for access flow measurements.

### VIII. Analysis

On a general level, the detection may involve calculating an evaluation parameter value based on the isolated pressure data resulting from the aforesaid signal extraction. The evaluation parameter value is then analysed as part of a process for determining flows of the cardiovascular system.

Different techniques for calculating such an evaluation parameter value are further disclosed and exemplified in Appendix B, in which the isolated pressure data is a time-dependent monitoring signal (denoted “filtered measurement signal”) which includes “second pulses” (heart pulses) originating from heart beats in the patient, and in which the monitoring signal is subjected to a time domain analysis. Thus, all techniques disclosed in Appendix B with respect to the evaluation of heart pulses, including the acquisition and use of “timing information”, are equally applicable to other physiological phenomena, such as breathing, autonomic regulation of body temperature, and autonomic regulation of blood pressure, or combinations thereof. Although the heart pulse or breathing are components in the present invention, the other of these two physiological phenomena or even further physiological phenomena may be included in the models to improve calculations, e.g. breathing signals. In addition to Appendix B, reference is also made to Applicant's International patent publication WO2009/156174, entitled “Methods and Devices for Monitoring the Integrity of a Fluid Connection”, which is incorporated herein in its entirety by this reference.

There are of course other techniques for calculating the evaluation parameter value, including other types of time domain analyses, as well as different types of frequency domain analyses, e.g. as indicated in the following.

Pressure data extracted from the measurement signal may be represented as a temporal pulse profile in the time domain. The temporal pulse profile may be transformed into a frequency spectrum and a phase spectrum, or only a frequency spectrum. From the pressure data, a parameter value may be calculated. The parameter value may be related to the amplitude, shape or timing of the pressure pulse, e.g. timing of a well-defined pulse feature such as zero-crossing or max/min occurrences.

FIG. 10 is a flow chart that illustrates an overview 550 of the process of determining fluid flows in a cardiovascular system using one or more pressure sensors in an extracorporeal system according to embodiments of the present invention. It shows an input of a measurement signal in 551 comprising pressure data as measured by one or more pressure sensors. Embodiments of the present invention may advantageously be described in terms of a model describing mathematical relations between flows and pressures of the circulatory system of a subject. It may be a generic model for any subject or individualized for a particular subject. The model may include the relationship between the pressure data extracted from the measurement signal and the corresponding pressure within the fistula. For instance, an initial model M₀ is defined 552 comprising model type and model definition parameters, i.e. model settings. The current model M for flow determination is set to the initial model M₀. Hence, the initial model is assigned to the current model M. The model settings may be pre-defined, generated based on information about the subject or a definition of the extracorporeal treatment, as is described below in connection with steps 559.

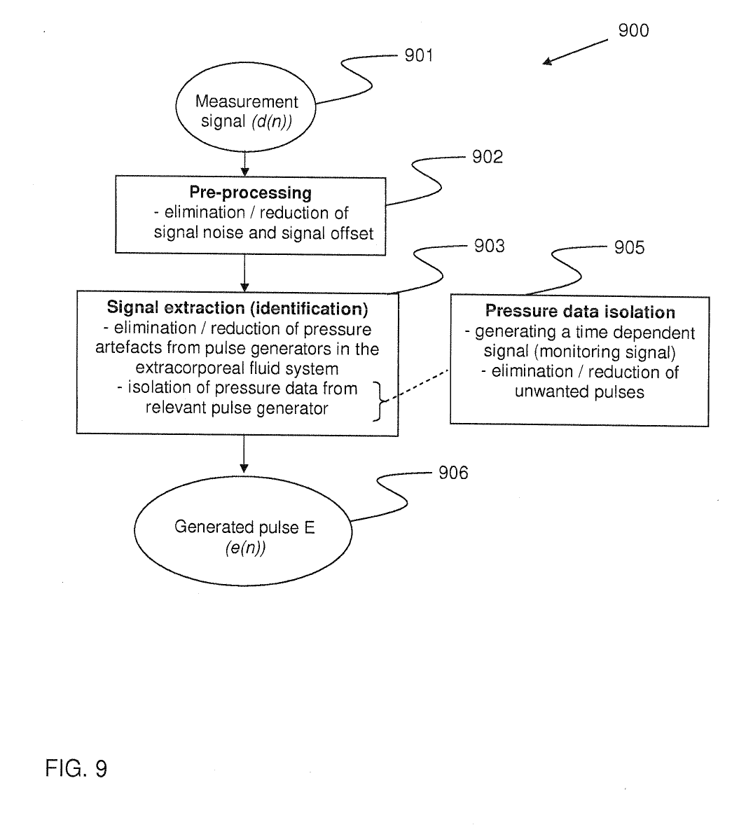

Next, a pulse measure E, i.e. pulse profile e(n), is generated 553 from the pressure measurement signal according to the procedure described in connection with FIG. 9 below. The pulse measure E is a temporal signal profile from which various features can be calculated. Based on E, a parameter P is generated 554, which parameter P relates to a fluid flow rate Q of the cardiovascular system. In the process of generating the parameter P, various features of E may be utilized, alone or in combination, such as amplitude, shape, frequency and/or phase or timing. The generation process may include mathematical operations such as peak-to-peak detection, integration, summation, variation (e.g. standard deviation), polynomial evaluation, etc. It may for instance involve the integral under the curve within a time window, sum of absolute or squared values within a time window or the difference between two pulses in the temporal signal profile. The relationship between the model M, pulse E and parameter P may be expressed as P=P(E), i.e. the parameter is a function of the pulse E, and Q=M(P), i.e. the fluid flow rate is a function of the model in which the parameter P is used, possibly in combination with additional parameters such as calibration C and blood pressure BP.

The parameter P may be an amplitude of the heart pulse. A cardiovascular flow rate may then be derived from linear or non-linear mathematical relationships. In an example of a non-linear relationship, a cardiovascular flow rate may be determined from a first relation between heart pulse amplitude Pₕ and fistula pressure P_{f}, such as the second order polynomial and curve 651 of FIG. 15, together with a second relation between the fistula pressure and access flow in the fistula. This relation and its connection with the model of FIG. 12 is further described in connection with section c) below. Over limited intervals, the non-linearity may further be approximated to a linear dependency. Measure points 652 indicate fistula pressure measurements to which the polynomial has been adjusted.

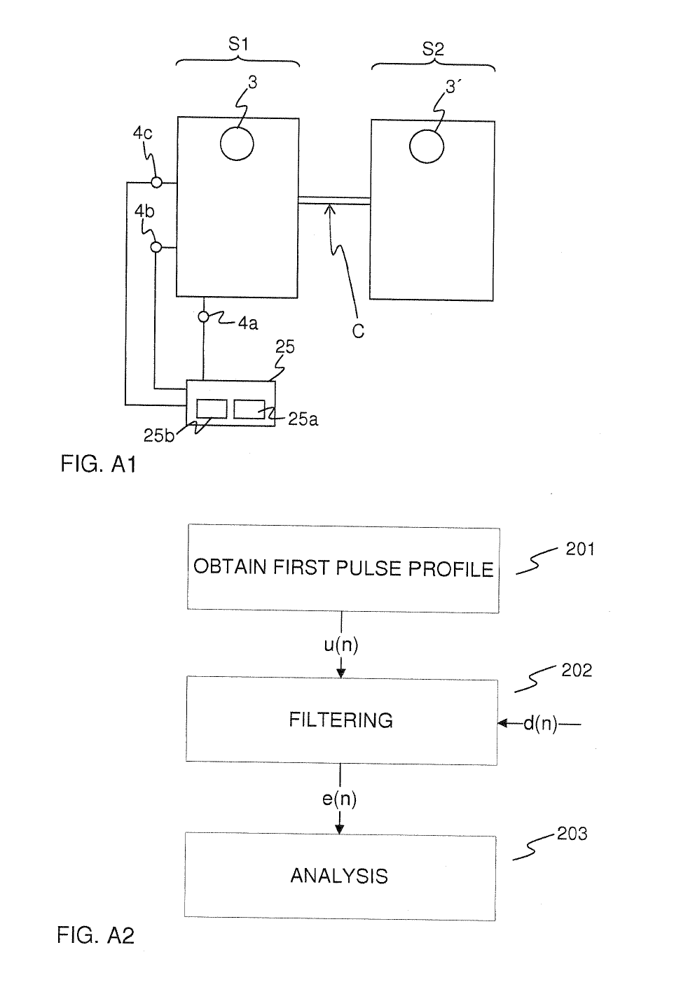

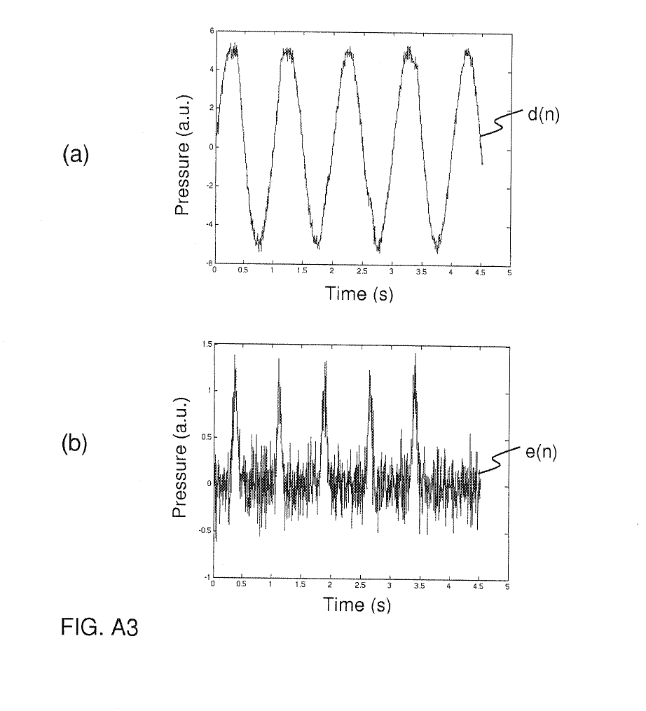

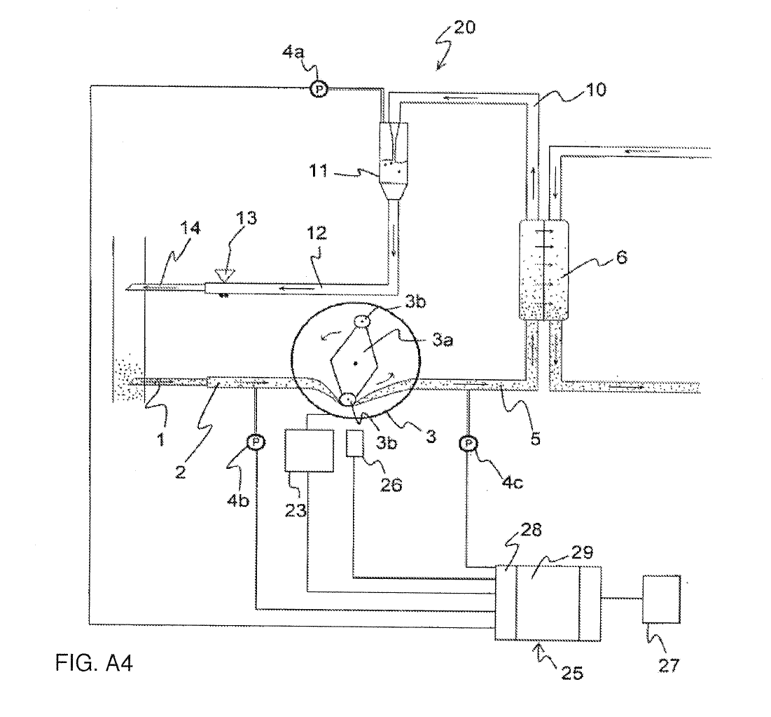

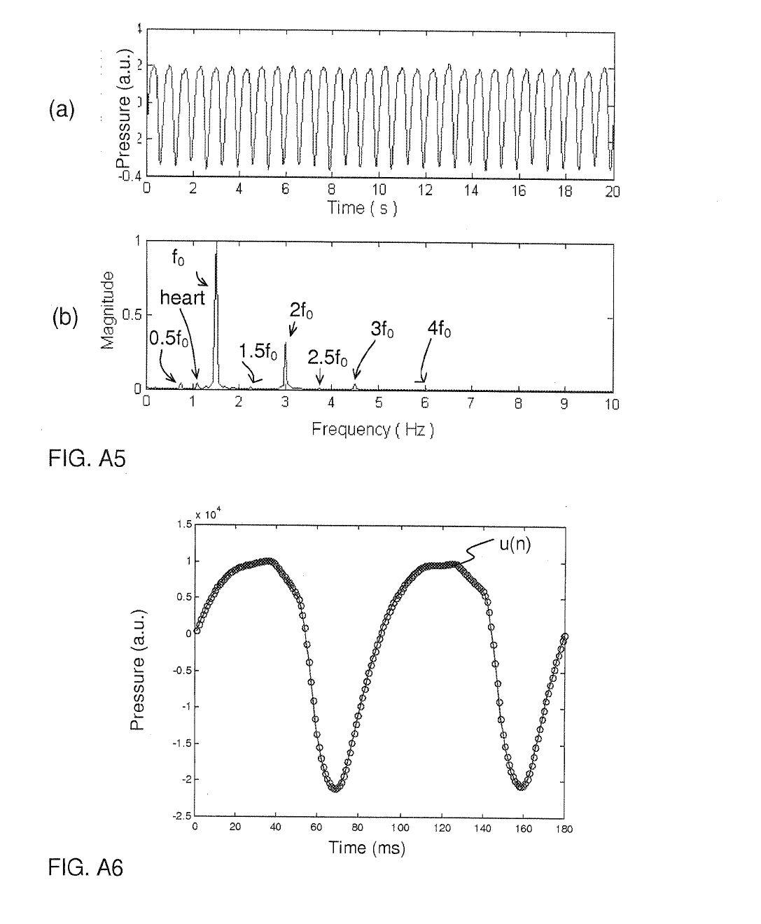

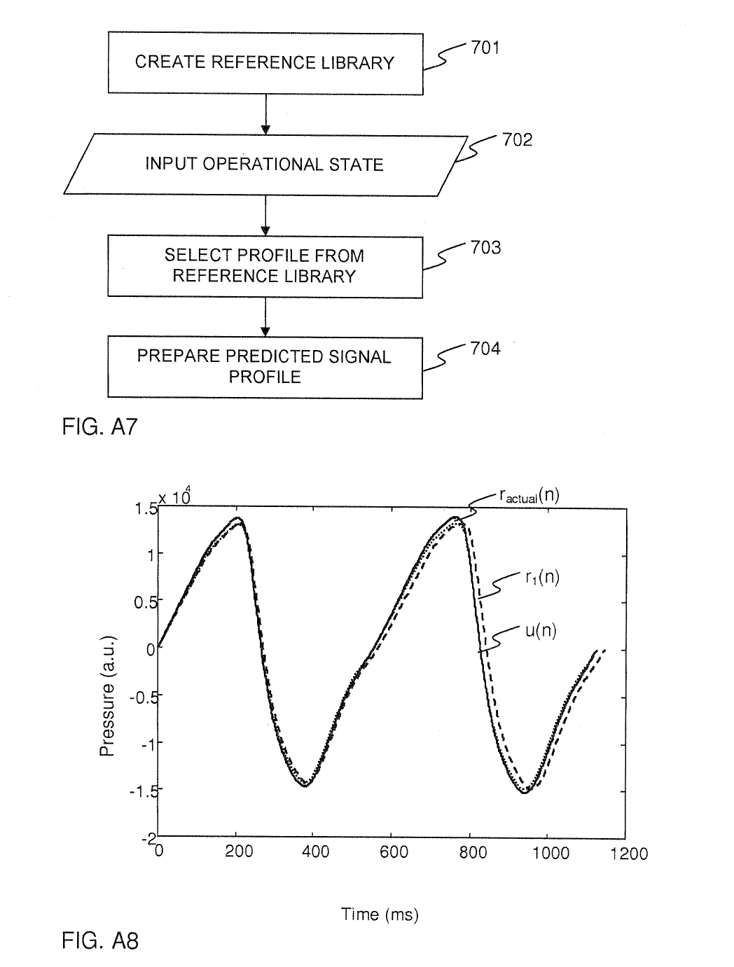

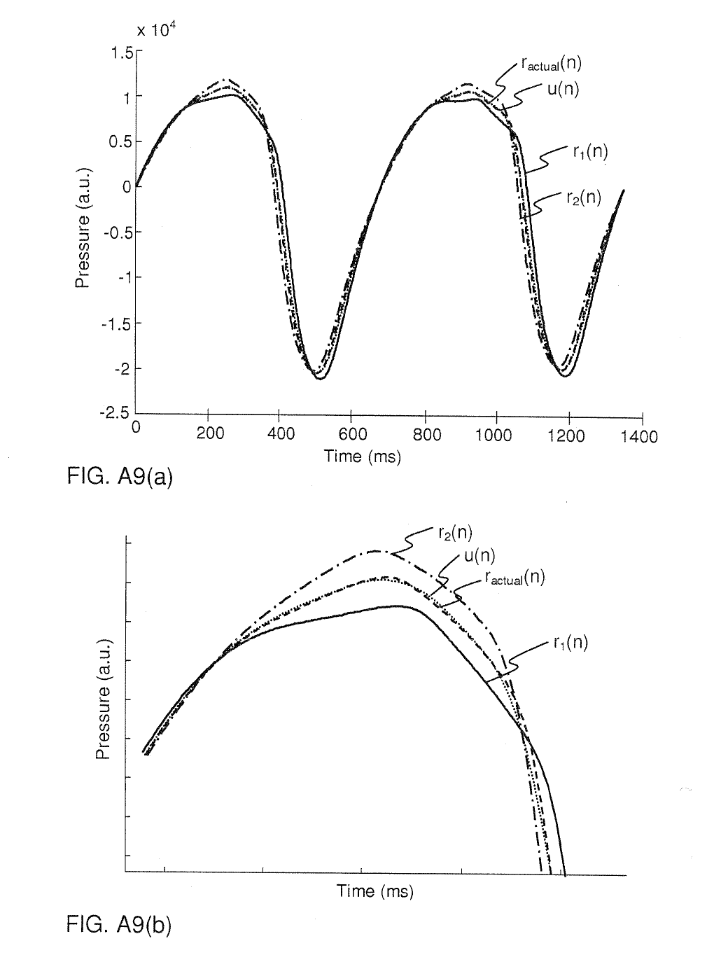

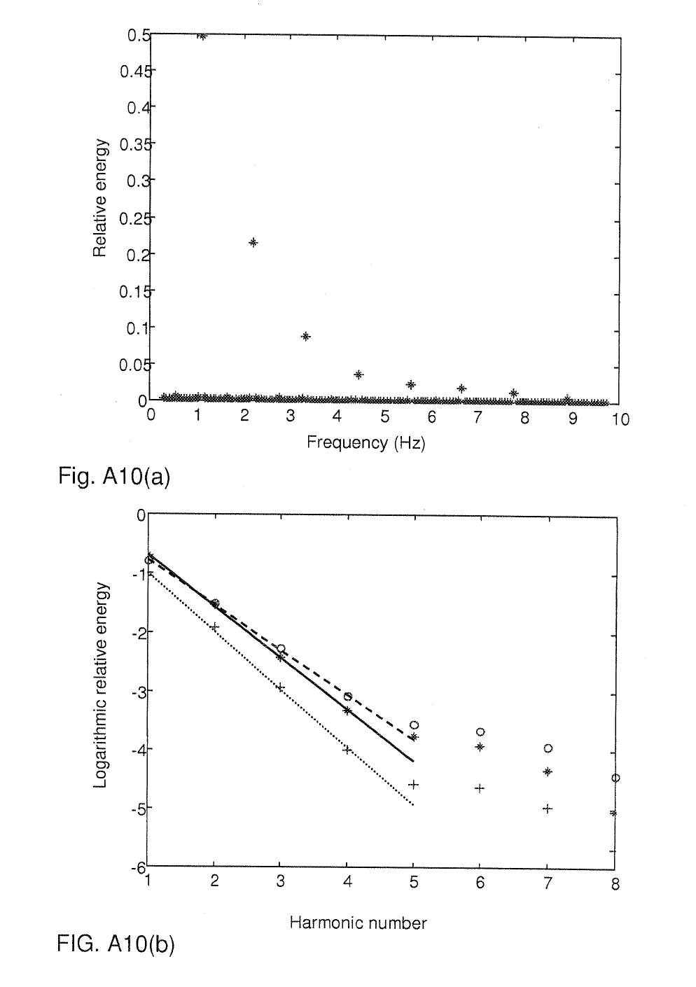

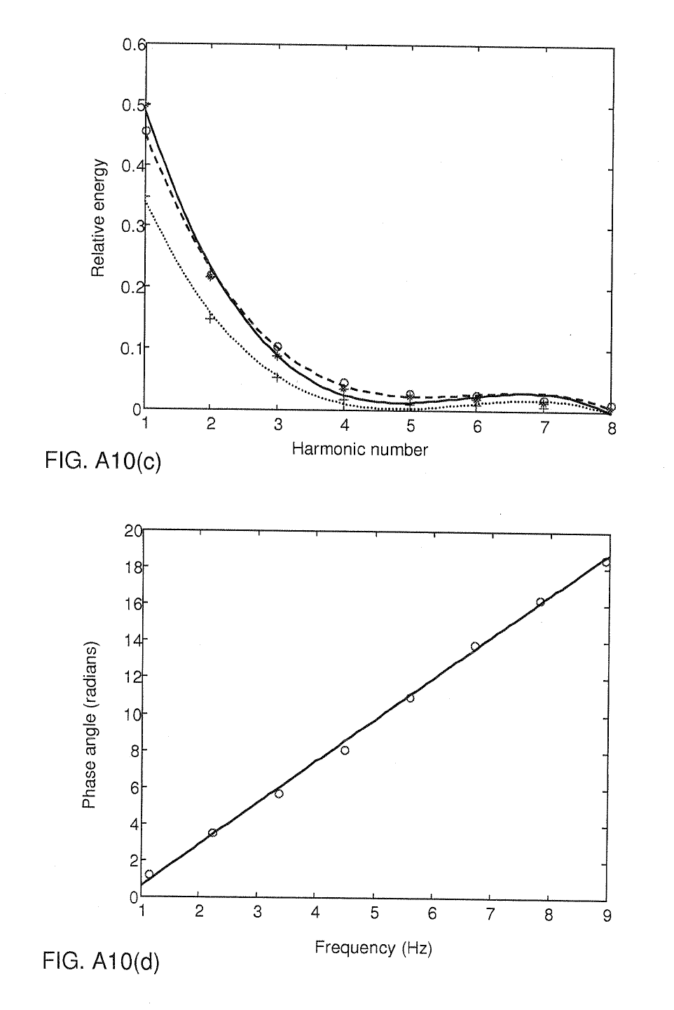

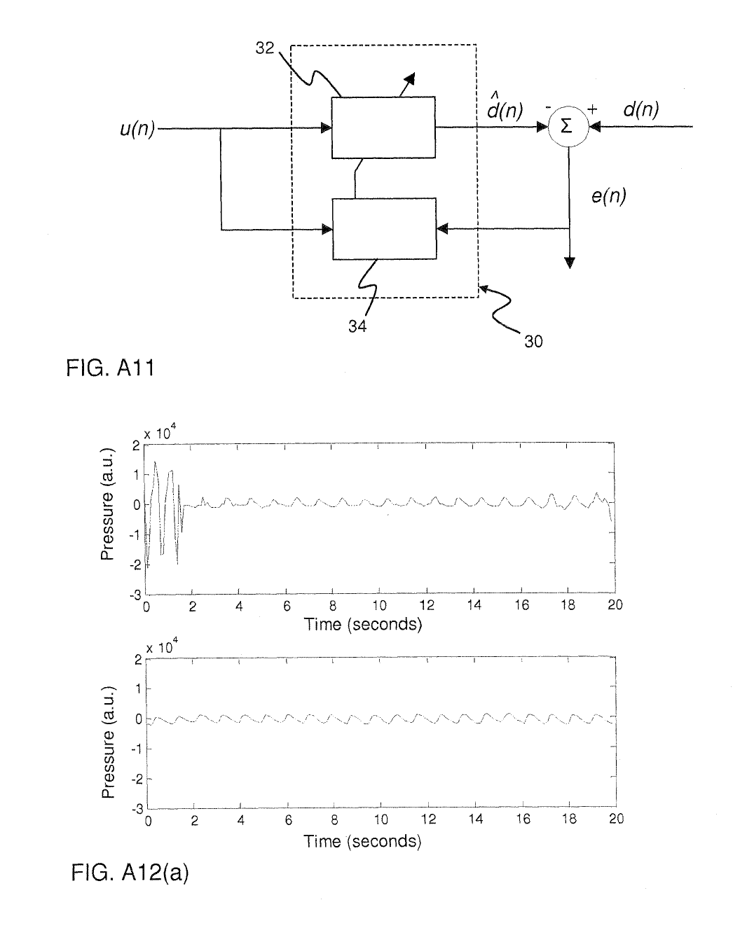

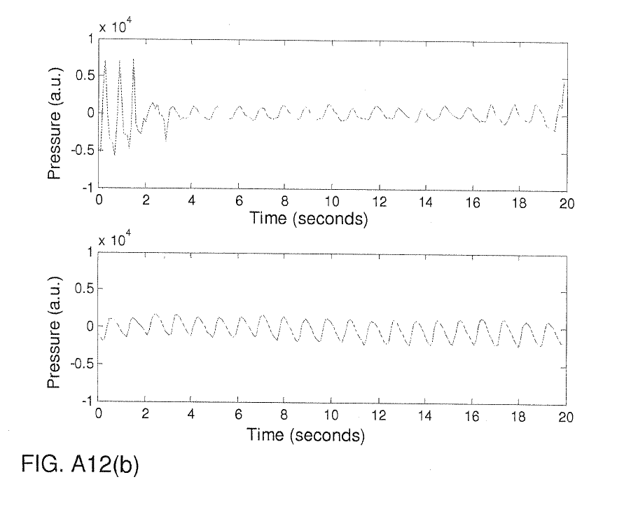

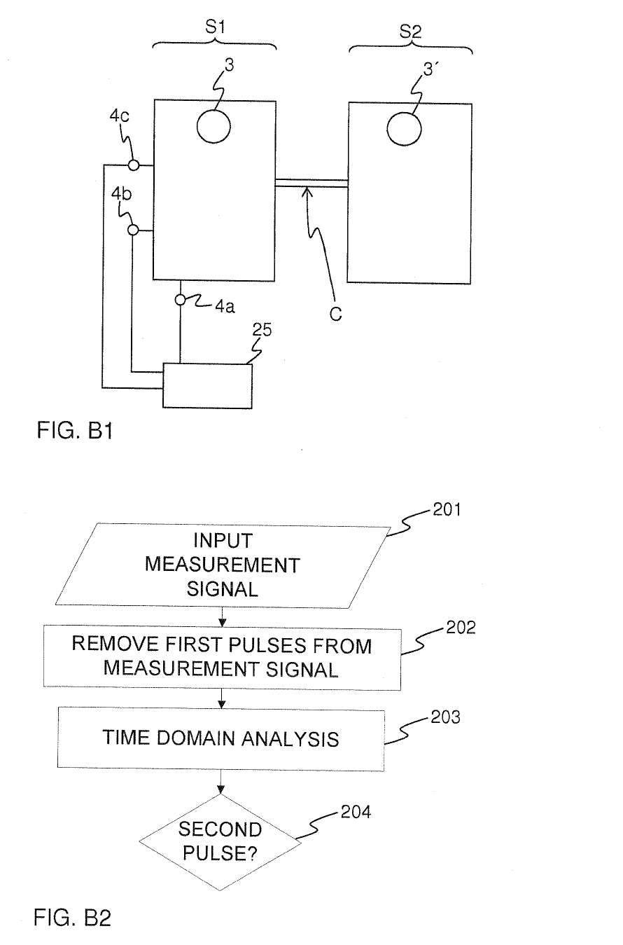

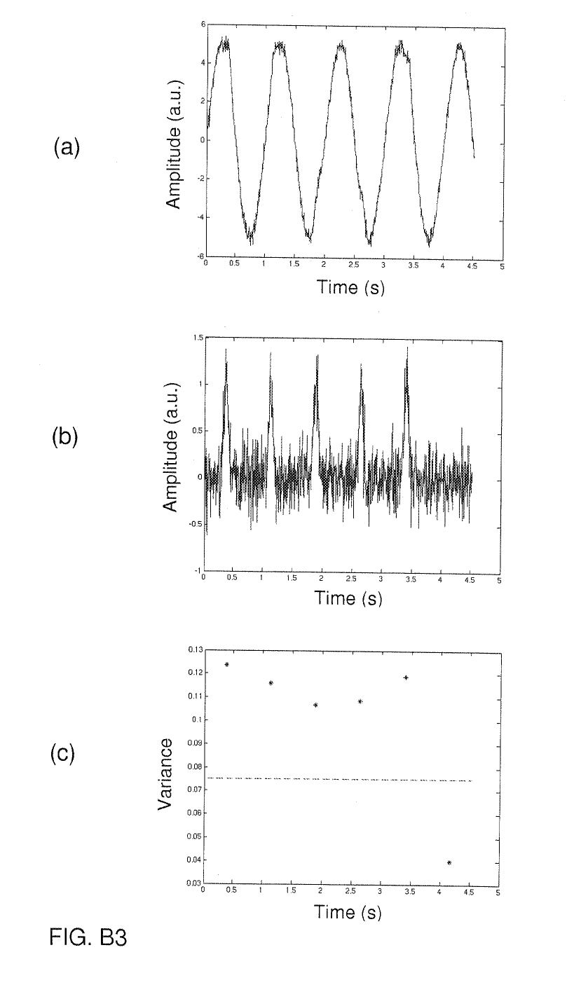

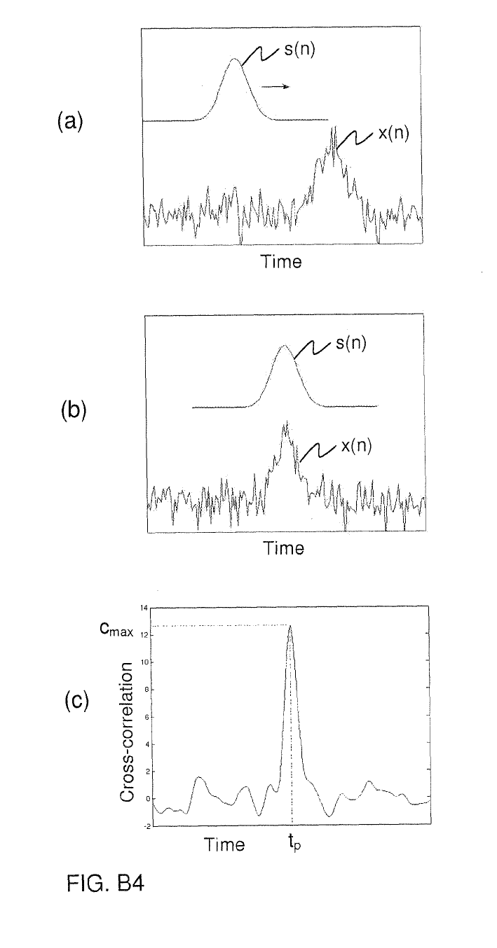

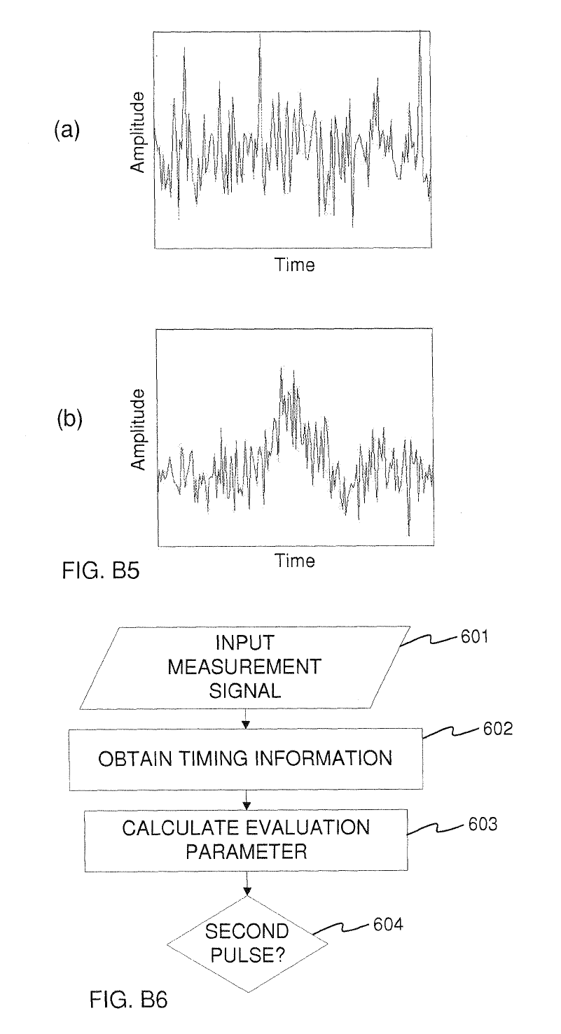

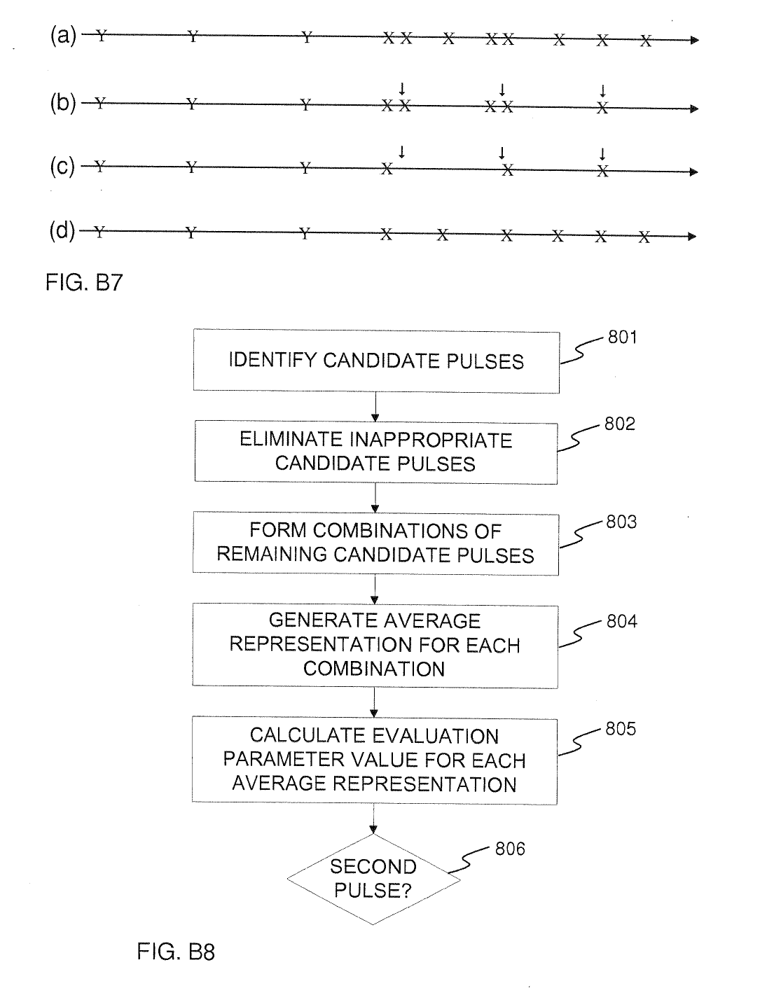

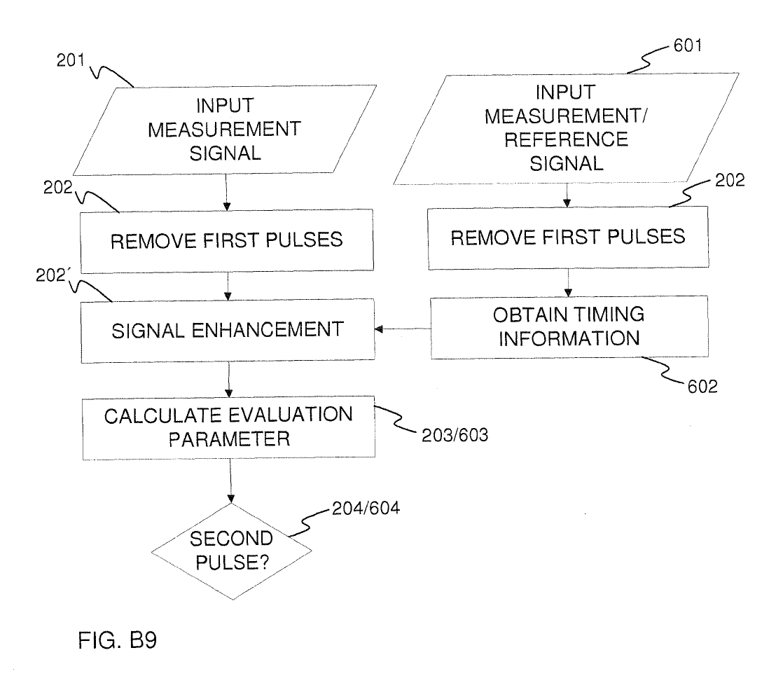

Alternatively, the parameter P may be generated based on shape data. Here, the parameter P may quantify the likeness of the pulse profile e(n) E to typical profiles representing high or low fluid flows, which may be illustrated with FIG. 16. FIG. 16 shows the two phases of a heart pulse waveform, i.e. the rising edges 661, 663 and falling edges 662, 664 of the pulse (the anacrotic and catacrotic phases). The heart pulse wave form from a vessel with a normal, moderate fluid flow rate show a “triphasic” shape as seen in section a) of FIG. 16. Higher blood pressure imply higher blood flow when the properties of the vascular system are essentially unchanged, meaning for instance that a pulse shape according to section b) of FIG. 16 represents a higher flow than the pulse shape of section a) of FIG. 16. The heart pulse wave form in a vessel with higher fluid flow rate tends to render the shape more “biphasic”, such as seen in section b) of FIG. 16. Note that the parameter P may be based on a plurality of pulse measure E values from e.g. a defined time period.

The fluid flow Q may then be calculated from the parameter P according to the model M as described in connection with step 559 below. Optionally, the fluid flow Q may be calibrated in a calibration step 555, in which flow calibration data C is obtained by performing one or more reference measurements of the flow. Depending on the model used one, two or more reference measurement may be required. A first order model requires one reference measurement if either the slope or the offset is known, two reference measurements are required if both slope and offset are unknown. Higher order models require additional calibration data. The calibration may be performed during the same or during different extracorporeal treatments for the same subject or possibly for a group of subjects.

For instance, in embodiments of the present invention, it is possible to obtain greater accuracy in absolute average values from reference measurements since we now know the timing of a particular reference measurement with respect to the flow determination of the present invention. Previously, when performing calibration, it has been assumed that the measurements have been on the curve of e.g. FIG. 7 or 8. For instance, it was not known if a reference measurement was obtained in a crest or a trough of the short time-scale variations. However, with the new knowledge of the short-time variations, it is seen that reference measurements are dependent on when in the variations measurements are obtained. Due to these variations, conventional reference methods based on isolated measurements depend on when measurements are performed. We now know when in short time-scale variations a particular reference measurement has been obtained, providing information as to how reference values relate to measurements on a longer time-scale. Embodiments of the present invention thus enable obtaining average values with less variability which may be used to improve the reference methods for obtaining relations to absolute values. The improvements relating to reference measurements benefit from particularly two aspects when having knowledge about the timing information. Firstly, the time of occurrence of reference measurements may be related to the occurrence of the relative measurements for a more precise determination of reference measurements. Secondly, the timing of reference measurements may be controlled with timing from the relative measurements according to the present invention. Since cardiovascular flow is affected by the blood pressure in the cardiovascular system, it may be relevant to include also blood pressure BP in the model to account for this effect, and thus improve the model. Blood pressure measurement may be performed in step 556. The measurement may obtain the Systolic, Diastolic, Mean Arterial Pressure MAP or a regional blood pressure of the subject. The then obtained current parameter P, calibration data C, blood pressure BP and previously recorded data such as information on the patient PD and the treatment TD may then be compared 557 with the current model M to check if the model is still valid for calculation of the fluid flow Q. The patient specific data PD and treatment specific data TD may have been recorded during the same treatment session or one or more previous treatment sessions.

If a satisfactory fit of the parameters C, P, BP, PD and TD with the model is achieved, the fluid flow Q is calculated in accordance with step 559. However, if a satisfactory fit cannot be achieved or combinations of the values of P, C, BP, PD and TD yield unlikely results with the model, the model is deemed “not valid” and the model is revised 558. Hence, a new or revised model is generated based on the same parameters C, P, BP, PD and TD. Optionally, new calibration measurements may be obtained. Also optionally, an alert “new model was required” may be issued and displayed. In case such an alert is given several times in sequence, a “not possible to adapt model”-message may be given to inform the operator that the flow measurement procedure may not be reliable. Alternatively, as a safety action, the measurement procedure may be aborted. Measurement readings, calibration data and the new model definition parameters may also be stored for subsequent use.

In fluid flow calculation step 559, the fluid flow Q is calculated based at least on the parameter P according to current model M. Other patient or treatment specific data (PD or TD) may also be provided 563 as input in the calculations. Three variations of models which may be used will now be briefly explained:
a) The model may be based upon a relative vascular flow measure R which is calculated based at least on the current parameter P and a normalization value Pi (P_initial), which for instance may be an initial fluid flow parameter value acquired at the beginning of a treatment. The relative flow measure R may be calculated as a ratio directly (P/Pi) or after a linear or non-linear transformation of P and Pi expressed as R=Q/Qi=M(P,Pi). Relative changes would be detected, such as stable or decreasing cardiac output as illustrated in FIG. 8 and FIG. 7 respectively.
b) The model may be based upon a fixed model of the absolute fluid flow Q versus the parameter P, i.e. Q=M_{fix}(P). Such a fixed model may have been obtained from assessment and/or comparison with measurement data recorded in previous treatment sessions of the same subject or a group of subjects. The model may be defined as any mathematical function, e.g. linear, non-linear etc, which produce Q based on at least the parameter P. The model may be fixed in the sense that it is unaltered between treatments. Determination of an absolute reference of the fluid flow Q may have been obtained in a previous treatment session.
c) The model may be a variable, tailored model which may be used for calculation of fluid flow Q based on at least the parameter P. The model may involve a plurality of model definition parameters which may be changed in real-time during a treatment and depending on individual and variable characteristics of the subject PD and changes of the treatment condition TD. The model may be linear such that the fluid flow may be calculated as Q=k*P+l, where k and l may be empirically determined from calibration data C. In another variant, the model may be given as a look-up table for different combinations of the parameter P and possibly other parameters such as blood pressure measurement BP, patient information data PD or treatment information data TD. For this purpose, it may be advantageous to obtain continuous blood pressure measurements, such as by using pressure wave velocity of arterial vessels which is disclosed and exemplified in the Applicant's provisional U.S. patent application No. 61/290,308 filed 28 Dec. 2009 entitled “MONITORING BLOOD PRESSURE” which was filed concurrently with the present application and which is incorporated herein in its entirety by this reference. According to another variant of the model, it may be given based on FIG. 12. Here, e.g. fluid flow Qₐ=Y₁(u₀−u₁), where u₀ is the artery pressure, u₁ is the fistula pressure and Y₁ is the flow admittance between point “0” and “1” in the diagram. Y₁ may be determined if a reference measurement of the fluid flow Qₐᵣ, e.g. with Doppler measurements or Cond-step methods, the fistula pressure u₁, and the artery pressure u₀ are known. FIG. 15 shows that the fistula pressure u₁, e.g. P_{f}, may be modelled based on the heart pulse amplitude measured in the extracorporeal circuit. Alternatively, also heart signal amplitude in the fistula may be derived and used as input for a model for determining the vascular flow. It is therefore possible to calculate the access flow Qa if the artery pressure value u₀ is given since both Y₁ and u₁ are known. Artery pressure u₀ may be obtained by blood pressure measurement. For this purpose, continuous blood pressure measurements may e.g. be achieved by combining an absolute blood pressure determination with continuous relative blood pressure measurements based on determination of pressure wave velocity of arterial vessels.

FIG. 9 is a flow chart that illustrates steps of a signal analysis process 900 according to an embodiment of the present invention. The process 900 operates on measurement data obtained (sampled) from, e.g., the venous, arterial and/or system pressure sensors, thereby generating signal values of a measurement signal comprising a number of pressure induced signal components.

The measurement signal comprises signals originating from one or more sources and thus constitutes a composite signal of the signals from said sources. The measurement signal may be used without further processing, although preferably, the measurement signal is processed for extraction of pressure data originating from a subject pulse generator in the cardiovascular system. The extraction may be performed by filtering to remove unwanted pressure data, such as from a blood pump in an extracorporeal system.

In the vascular system, a subject pulse generator may be a physiological phenomenon, such as the pulsations of from the heart or breathing from the lungs. The signal analysis process may be divided into a pre-processing part 902, a signal extraction part 903 and an analysis part 904. The pre-processing part 902 includes elimination or reduction of signal noise, e.g. measurement noise, and signal offset, e.g. as detailed in the section above relating to the data acquisition part 28. The signal extraction part 903 involves elimination or reduction of pressure artefacts originating from pulse generators in the extracorporeal fluid circuit or in the cardiovascular system and isolation of pressure data originating from the relevant physiological phenomenon. In the context of the present disclosure, “pressure data isolation” 905 denotes a process of generating a time-dependent signal profile of the subject pulse (e(n)) which is free or substantially free from pressure modulations caused by any unwanted physiological phenomena. Such unwanted physiological phenomena may vary between different applications, and may include breathing, coughing, etc. The elimination of signal noise and signal offset, as well as the elimination of pressure artefacts, may be included in algorithms for pressure data isolation. For instance, the measurement signal may be band pass filtered or low pass filtered to isolate a heart signal, in a way such that signal noise and/or signal offset and/or pressure artefacts are eliminated from the measurement signal. The elimination of pressure artefacts may thus be performed before, after or during the pressure data isolation.

The generated pulse measure E (e(n)) is input in step 553 in FIG. 10 to generate a parameter P, as further described in connection with FIG. 10.

The calculation may be designed such that the parameter value represents timing, amplitude or shape of the pulse. However, the detection may also be performed in the frequency domain by analysis of the amplitude and/or phase spectrum.

In the simplest case of pressure signal analysis, no pump or other source of pressure artefacts is present in the extracorporeal fluid circuit connected to the subject during the data acquisition. For instance, the pump may have been shut down.

In the general case, however, one or more pumps are running or other sources of cyclic or non-cyclic repetitive and non-repetitive artefacts are present during the data acquisition. Information on the cyclic disturbances may be known from external sources, e.g. other sensors, or may be estimated or reconstructed from system parameters.

Cyclic pressure artefacts may originate from operating a peristaltic pump, repetitive actuation of valves, movements of membranes in balancing chambers. According to the findings in connection with the present invention, artefacts may also originate from mechanical resonance of system components such as swinging movements of blood line energized by e.g. a pump. Frequencies of blood line movements are given by the tube lengths and harmonics thereof and by the beating between any frequencies involved, i.e. between different self-oscillations and pump frequencies. These frequencies may differ between the venous and arterial lines. Mechanical fixation of the blood lines and other free components may remedy the problem of mechanical resonance. Alternatively, an operator may be instructed to touch or jolt the blood lines to identify natural frequencies associated with the blood lines, which information may be used in the analysis for improved removal of components not belonging to the pressure data of interest.

Examples of non-cyclic artefacts are subject movement, valve actuation, movements of tubings etc.

Various techniques for signal extraction will be discussed in a section further below.

The invention has mainly been described above with reference to a few embodiments. However, as is readily appreciated by a person skilled in the art, other embodiments than the ones disclosed above are equally possible within the scope and spirit of the invention, which is defined and limited only be the appended patent claims.

For example, the illustrated embodiments are applicable for cardiovascular flow measurements involving all types of extracorporeal blood flow circuits in which blood is taken from a patient's circulation. Such blood flow circuits include hemodialysis, hemofiltration, hemodiafiltration, plasmapheresis, aphaeresis, extracorporeal membrane oxygenation, assisted blood circulation, and extracorporeal liver support/dialysis, bloodbanking, blood fraction separation (e.g. cells) of donor blood, etc.

Further, the inventive monitoring techniques are applicable to any type of pumping device that generates pressure pulses in the first fluid containing system, not only rotary peristaltic pumps as disclosed above, but also other types of positive displacement pumps, such as linear peristaltic pumps, diaphragm pumps, as well as centrifugal pumps.

Tests have shown that different evaluation parameters may be preferable in different situations. For example, the use of variance or averaged sum of multiple pulses may increase the accuracy and/or detectability in various situations. Pattern recognition may be resorted when other detection methods fail.

Furthermore, although it may generally be enough for evaluation purposes to involve one parameter value, it may be advantageous to base an evaluation on combinations of two or more parameter values, such as to improve the reliability of the flow determination. The reliability may also be enhanced by increasing the evaluation time period. It may further be advantageous to increase the resolution in the measurements to further improve the reliability.

Although the model has been described with examples involving the heart pulses as primary source for analysis, the breathing of a subject may also be used as a primary source for analysis. As with heart pulses, breathing also constitutes a modulator of cardiovascular flow rates Q and similar parameters P may be used for measuring the for instance amplitude, shape, phase and/or frequency on the temporal signal profile.

### IX. Signal Extraction

In the following, embodiments for eliminating or reducing various pressure artefacts (also denoted “pump pulses” or “interference pulses”) originating from one or more pulse generators in or associated with extracorporeal circuit will be described. Then, embodiments for isolating pressure data originating from a relevant physiological phenomenon among pressure pulses or pressure modulations originating from other physiological phenomena are described.

The pressure data to be extracted is not limited to a single physiological phenomenon and may originate from one or more physiological phenomena, including the heart. As used herein, the pressure data to be isolated is also denoted “subject pulses” or “patient pulses”.

### Elimination of Artefacts

Elimination of artefacts may be provided by:
Controlling a pulse generator in the extracorporeal fluid system, such as a pump
   By temporarily shutting down the pulse generator;
   Shifting the frequency of the pulse generator;
Low pass, band pass or high pass filtering;
Spectral analysis and filtering in the frequency domain;
Time domain filtering.

### Controlling a Pulse Generator

Artefacts from a pulse generator, such as a pump, in the extracorporeal fluid circuit may be avoided by temporarily shutting down (disabling) the pulse generator, or by shifting the frequency of the pulse generator away from frequencies of one or more relevant physiological phenomena. A feedback control with respect to the heart rate, e.g. obtained from a dedicated pulse sensor attached to the patient or obtained via analysis of previous parts of the monitoring signal, may be used to set the pump frequency optimally for detection of heart pulses. Similar feedback control may be used to eliminate artefacts with respect to pressure pulses originating from breathing, e.g. based on a breathing signal from an independent source, such as a capnograph instrument. Hence, the control unit 23 of FIG. 1 may be operated to control the pump frequency in order to facilitate the detection of the subject pulses, e.g. the pump frequency is controlled to minimize any overlap in frequency between the pump pulses and the subject pulses. For example, the pump frequency may be periodically increased and decreased around the overlap frequency, so as to maintain the overall blood flow rate. In a variant, the pump frequency is instead controlled so as to synchronize the rate of pump pulses with the rate of subject pulses while applying a phase difference between the pump pulses and the subject pulses. Thereby, the pump pulses and the subject pulses will be separated in time, and the subject pulses may be detected in the time domain, even without removal of the pump pulses. The phase difference may be approximately 180°, since this may maximize the separation of the pump pulses and the subject pulses in the time domain. This so-called phase-locking technique may be activated when it is detected that the rate of subject pulses approaches the rate of pump pulses, or vice versa.

### Applying Low Pass, Band Pass or High Pass Filters

The input signal to step 903 (FIG. 9) may be fed into a filter, e.g. digital or analogue, with frequency characteristics, such as frequency range and/or centre of frequency range, matched to the frequencies generated by a pulse generator, such as a pump, in the extracorporeal circuit. For instance, in a case where the pulse generator, such as a pump, operates within the frequency range of 1 Hz, a suitable low pass filter may be applied in order to remove pressure artefacts above 1 Hz while retaining frequency components of the physiological phenomenon below 1 Hz. Correspondingly, a high pass filter may be applied to retain frequency components above the frequency of the pulse generator. Alternatively, one or more notch filters or the like may be utilised to remove/attenuate frequencies in one or more confined ranges.

### Spectral Analysis and Filtering in the Frequency Domain

The input signal to part 133 may be subjected to a spectral analysis, e.g. by applying a Fourier transformation technique, such as FFT (Fast Fourier Transform) to convert the input signal into the frequency domain. The resulting energy spectrum (amplitude spectrum) may then be multiplied by an appropriate filter function and then re-transformed into the time domain. There are many alternative and equivalent filtering techniques available to the skilled person.

### Time Domain Filtering

Artefact elimination by filtering in the time domain is further disclosed and exemplified in Appendix A. In the context of Appendix A, the input signal to step 903 (FIG. 9) is denoted “measurement signal”, and the resulting “filtered signal e(n)” corresponds to, or may be processed for extraction of, the above-mentioned pulse E. In addition to Appendix A, reference is also made to Applicant's International patent publication WO2009/156175, entitled “Method and device for processing a time-dependent measurement signal”, which is incorporated herein in its entirety by this reference.

Isolating Pressure Data from a Physiological Phenomenon

Isolating pressure data originating from a relevant physiological phenomenon may be provided by any or a combination of:
Low pass, band pass or high pass filtering;
Spectral analysis and filtering in the frequency domain; or
Time domain filtering.

### Applying Low Pass, Band Pass or High Pass Filters

The input signal to step 905 may be fed into a filter, e.g. digital or analogue, with frequency characteristics, such as frequency range and/or centre of frequency range, matched to the frequencies of pressure pulses from a relevant physiological phenomenon where e.g. in case the isolation concerns:
Heart pulses, a frequency range substantially of 0.5-4 Hz will be allowed to pass the filter;
Breathing, a frequency range substantially of 0.15-0.4 Hz will be allowed to pass the filter;
Blood pressure regulation due to the autonomous system, a frequency range substantially of 0.04-0.15 Hz will be allowed to pass the filter; and
Temperature regulation due to the autonomous system, a frequency range substantially of 0.001-0.1 Hz will be allowed to pass the filter.

### Spectral Analysis and Filtering in the Frequency Domain

The input signal to step 905 may be subjected to a spectral analysis, e.g. by applying a Fourier transformation technique, such as FFT (Fast Fourier Transform) to convert the input signal into the frequency domain. The resulting energy spectrum (amplitude spectrum) may then be multiplied by an appropriate filter function and then re-transformed into the time domain. There are many alternative and equivalent filtering techniques available to the skilled person.

### Time Domain Filtering

The signal of interest may be extracted from the input signal to step 905 as an error signal of an adaptive filter. The adaptive filter is fed with both the measured pressure signal and a predicted signal profile of a cyclic disturbance. The cyclic disturbance may originate from any unwanted physiological phenomenon (e.g. heart pulsation or breathing). Particularly, a reconstructed pressure profile originating from the unwanted physiological phenomenon may be input to the adaptive filter. This and other time domain filtering techniques for removing unwanted signal components from a measurement signal is further disclosed and exemplified in Appendix A. Although Appendix A is concerned with eliminating first pulses originating from a pulse generator in an extracorporeal circuit, such as a pumping device, it is equally applicable for eliminating first pulses originating from unwanted physiological phenomena, as long as a predicted signal profile of the first pulses may be obtained. The skilled person realizes that such a predicted signal profile may be obtained in any of the ways described in Appendix A. In addition to Appendix A, reference is also made to aforesaid WO2009/156175.

Some of the filtering techniques described above may automatically be achieved by down-sampling, since it may be taken care of by the anti-aliasing filter included in a down-sampling signal processing algorithm. Additionally, some of the above described filtering techniques may also be achieved directly in hardware, e.g., in the Analogue-to-Digital conversion by choosing an appropriate sample frequency, i.e. due to the anti-aliasing filter which is applied before sampling.

The term “comprises/comprising” when used in this specification is taken to specify the presence of stated features, integers, steps or components. However, the term does not preclude the presence or addition of one or more additional features, integers, steps or components or groups thereof.

It is to be understood that Appendix A and Appendix B are to be treated as integral parts of the present application. However, reference numerals are defined within the context of each Appendix separately. In the event of conflicting use of terminology between the Appendix A, Appendix B and the main specification, the terminology should be interpreted within the context of Appendix A, Appendix B and the main specification, respectively.

The invention is not restricted to the described embodiments in the figures, but may be varied freely within the scope of the claims.

In the following, a set of items are recited to summarize some aspects and embodiments of the invention as disclosed in the foregoing.

Item 1.

A device for monitoring a fluid flow rate (Q) of a cardiovascular system of a subject, said the device comprising an input (28) for obtaining a time-dependent measurement signal (d(n)) from a pressure sensor (4a-4c) in an extracorporeal blood circuit (20) which is adapted for connection to the cardiovascular system, the pressure sensor (4a-4c) being arranged to detect a subject pulse originating from a subject pulse generator (3′) in the cardiovascular system of the subject, wherein the device further comprises a signal processor (29) connected to the input (28) and being configured to: process the measurement signal to obtain a pulse profile (e(n)) which is a temporal signal profile of the subject pulse, and calculate the fluid flow rate (Q) based at least partly on the temporal signal profile.

Item 2.

The device according to item 1, wherein the subject pulse generator (3′) is a part of the cardiovascular system.

Item 3.

The device according to item 1 or 2, wherein the extracorporeal blood circuit (20) comprises a fluid pathway (10), a blood processing device (6), and at least one pumping device (3), and wherein the pressure sensor (4a-4c) is further arranged to detect a pump pulse originating from the pumping device (3).

Item 4.

The device according to item 3, wherein each pulse profile (e(n)) corresponds to a time window in the measurement signal.

Item 5.

The device according to item 4, wherein the signal processor (29) is adapted to set the time window based on timing information indicative of a timing of subject pulses in the measurement signal.

Item 6.

The device according to item 5, wherein the signal processor (29) is adapted to obtain the timing information from a pulse sensor coupled to the subject.

Item 7.

The device according to item 5, wherein the signal processor (29) is adapted to obtain the timing information as a function of the relative timing of previously identified subject pulses in the measurement signal.

Item 8.

The device according to item 5, wherein the signal processor (29) is adapted to: identify a set of candidate subject pulses in the measurement signal; derive a sequence of candidate time points based on the set of candidate subject pulses; validate the sequence of candidate time points against a temporal criterion; and calculate the timing information as a function of the thus-validated sequence of candidate time points.

Item 9.

The device according to item 5, wherein the signal processor (29) is adapted to: intermittently turn off the pumping device (3); identify at least one subject pulse in the measurement signal; and calculate the timing information from the thus-identified subject pulse.

Item 10.

The device according to any one of items 3-9, wherein the signal processor (29) is further adapted to generate, based on the measurement signal, a time-dependent monitoring signal in which the pump pulse is essentially eliminated, whereupon the signal processor (29) obtains the pulse profile (e(n)) from the monitoring signal.

Item 11. The device according to item 10, wherein the signal processor (29) is further adapted to generate the monitoring signal by: filtering the measurement signal to remove the pump pulse; deriving, based on timing information indicative of the timing of the subject pulses in the measurement signal, a set of signal segments in the thus-filtered measurement signal; and aligning and adding the signal segments, based on the timing information, to generate the monitoring signal.

Item 12.

The device according to item 10 or 11, wherein the signal processor (29) is adapted to obtain a pump profile (u(n)) which is a predicted temporal signal profile of the pump pulse, and to filter the measurement signal in the time domain, using the pump profile (u(n)), to essentially eliminate the pump pulse while retaining the subject pulse.

Item 13.

The device according to item 12, wherein the signal processor (29) is adapted to subtract the pump profile (u(n)) from the measurement signal.

Item 14.

The device according to item 13, wherein the signal processor (29) is adapted to, before subtracting the pump profile (u(n)), adjust at least one of the amplitude, the time scale and the phase of the pump profile (u(n)) with respect to the measurement signal.

Item 15.

The device according to item 14, wherein the signal processor (29) is adapted to minimize a difference between the pump profile (u(n)) and the measurement signal.

Item 16.

The device according to any one of items 13-15, wherein the signal processor (29) is adapted to subtract the pump profile (u(n)) by adjusting a phase of the pump profile (u(n)) in relation to the measurement signal, wherein said phase is indicated by phase information obtained from at least one of: a pump rate sensor (25) coupled to the pumping device (3), and a controller (24) for the pumping device (3).

Item 17.

The device according to item 12, wherein the signal processor (29) comprises an adaptive filter (30) which is arranged to generate an estimation signal (??\hat{d}(n)), based on the pump profile (u(n)) and an error signal (e(n)) formed as a difference between the measurement signal and the estimation signal (??\hat{d}(n)), whereby the adaptive filter (30) is arranged to essentially eliminate the pump pulse in the error signal (e(n)). Further, the adaptive filter (30) may be configured to generate the estimation signal (\hat{d}(n)) as a linear combination of M shifted pump profiles (u(n)), and specifically the adaptive filter (30) may be configured to linearly combine M instances of the pump profiles (u(n)), which are properly adjusted in amplitude and phase by the adaptive filter (30).

Item 18.

The device according to item 17, wherein the adaptive filter (30) comprises a finite impulse response filter (32) with filter coefficients that operate on the pump profile (u(n)) to generate the estimation signal (??\hat{d}(n)), and an adaptive algorithm (34) which optimizes the filter coefficients as a function of the error signal (e(n)) and the pump profile (u(n)).

Item 19.

The device according to item 17 or 18, wherein the signal processor (29) is adapted to control the adaptive filter (30) to lock the filter coefficients, based on a comparison of the rate and/or amplitude of the subject pulses to a limit value.

Item 20.

The device according to any one of items 12-19, wherein the signal processor (29) is adapted to, in a reference measurement, cause the pumping device (3) to generate at least one pump pulse, and obtain the pump profile (u(n)) from a reference signal generated by a reference sensor (4a-4c). The reference sensor may be a pressure sensor in the extracorporeal blood circuit. The extracorporeal blood circuit may be operated, during the reference measurement, such that the reference signal contains a pump pulse and no subject pulse.

Item 21.

The device according to item 20, wherein the pumping device (3) is operated to generate a sequence of pump pulses during the reference measurement, and wherein the pump profile (u(n)) is obtained by identifying and combining a set of pump pulses in the reference signal.

Item 22.

The device according to item 20 or 21, wherein the signal processor (29) is adapted to intermittently effect the reference measurement to update the pump profile (u(n)) during operation of the extracorporeal blood circuit (20).

Item 23.

The device according to any one of items 20-22, wherein the signal processor (29) is adapted to effect the reference measurement by: obtaining a combined pulse profile based on a first reference signal containing a pump pulse and a subject pulse; obtaining a subject pulse profile based on a second reference signal containing a subject pulse and no pump pulse, and obtaining the pump profile by subtracting the subject pulse profile from the combined pulse profile.

Item 24.

The device according to any one of items 12-19, wherein the signal processor (29) is adapted to obtain the pump profile (u(n)) based on a predetermined signal profile.

Item 25.

The device according to item 24, wherein the signal processor (29) is adapted to modify the predetermined signal profile according to a mathematical model based on a current value of one or more system parameters of the extracorporeal blood circuit (20).

Item 26.

The device according to any one of items 12-19, wherein the signal processor (29) is adapted to obtain a current value of one or more system parameters of the extracorporeal blood circuit (20), and to obtain the pump profile (u(n)) as a function of the current value.

Item 27.

The device according to item 26, wherein the signal processor (29) is adapted to obtain the pump profile (u(n)) by identifying, based on the current value, one or more temporal reference profiles (r₁)(n), r₂(n)) in a reference database; and obtaining the pump profile (u(n)) based on said one or more temporal reference profiles (r)₁(n), r₂(n)).

Item 28.

The device according to item 27, wherein said one or more system parameters is indicative of a pumping rate of the pumping device (3).

Item 29.

The device according to item 27 or 28, wherein each temporal reference profile (r)₁(n), r₂(n)) in the reference database is obtained by a reference measurement in the extracorporeal blood circuit (20) for a respective value of said one or more system parameters.

Item 30.

The device according to item 29, wherein the signal processor (29) is adapted to obtain the pump profile (u(n)) by identifying, based on the current value, one or more combinations of energy and phase angle data in a reference database; and obtaining the pump profile (u(n)) based on said one or more combinations of energy and phase angle data.

Item 31.

The device according to item 30, wherein the signal processor (29) is adapted to obtain the pump profile (u(n)) by combining a set of sinusoids of different frequencies, wherein the amplitude and phase angle of each sinusoid is given by said one or more combinations of energy and phase angle data.

Item 32.

The device according to item 26, wherein the signal processor (29) is adapted to obtain the pump profile (u(n)) by inputting the current value into an algorithm which calculates the response of the pressure sensor (4a-4c) based on a mathematical model of the extracorporeal blood circuit (20).

Item 33.

The device according to any one of items 9-32, wherein the signal processor (29) is further adapted to calculate a rate of subject pulses in the measurement signal, or in a reference signal obtained from a reference sensor (4a-4c), and to cause a pumping frequency of the pumping device (3) to be controlled in relation to the rate of subject pulses.

Item 34.

The device according to item 33, wherein the pumping frequency is controlled to shift the rate of pump pulses away from the rate of subject pulses.

Item 35.

The device according to item 33, wherein the pumping frequency is controlled to synchronize the rate of pump pulses with the rate of subject pulses, while applying a given phase difference between the pump pulses and the subject pulses.

Item 36.

The device according to any one of items 9-32, wherein the signal processor (29) is adapted to obtain the pulse profile (e(n)) while the pumping device (3) is intermittently set in a disabled state.

Item 37.

The device according to any preceding item, wherein the subject pulse generator (3′) is any of the heart, the breathing system, or any combinations thereof.

Item 38.

The device according to any preceding item, wherein the calculating of the fluid flow rate (Q) involves one or more of amplitude, shape, and timing of the temporal signal profile.

Item 50.

A method for monitoring a fluid flow rate (Q) in a cardiovascular system of a subject, said method comprising: obtaining a time-dependent measurement signal (d(n)) from a pressure sensor (4a-4c) in an extracorporeal blood circuit (20) which is arranged in fluid connection with the cardiovascular system, the pressure sensor (4a-4c) being arranged to detect a subject pulse originating from a subject pulse generator (3′), processing the measurement signal to obtain a pulse profile (e(n)) which is a temporal signal profile of the subject pulse, and calculating a fluid flow rate (Q) based at least partly on the temporal signal profile.

Item 51.

The method according to item 50, further comprising varying a blood flow (i2) of the extracorporeal blood circuit (20).

Item 52.

The method according to item 50 or 51, further comprising aggregating a plurality of pulse profiles within an aggregation time window in the measurement signal and calculating the fluid flow rate (Q) based on an average of the plurality of the pulse profiles.

Item 53.

The method according to any one of items 50-52, wherein the calculating involves calculation of the cardiac output (CO) of the cardiovascular system.

Item 54.

The method according to any one of items 50-52, wherein the calculating involves calculation of an access flow (Qa) of a blood access in the cardiovascular system.

Item 55.

The method according to any one of items 50-54, further comprising calibrating the fluid flow rate (Q) against one or more calibration values.

Item 56.

The method according to item 55, wherein the calibration comprises: providing a detectable perturbation to at least a measurable blood characteristic in the cardiovascular system, measuring an integrated change of a corresponding characteristic on a treatment fluid outlet of the extracorporeal blood circuit (20), and determining the fluid flow rate (Q) based on the measurement of said integrated change of the treatment fluid outlet.

Item 57,

The method according to item 55, wherein the calibration comprises: obtaining a first conductivity or concentration measurement in a treatment fluid of the extracorporeal blood circuit (20) running in a first direction, obtaining a second conductivity or concentration measurement in the treatment fluid running in a second direction, and calculating the access flow rate (Qₐ) in said blood access as a function of: said first conductivity or concentration measurement and of said second conductivity or concentration measurement.

Item 58.

The method according to item 50, further comprising calculating an access flow rate Qa and an associated variance QaV, retrieving a withdrawal blood flow rate Qb, and in case the sum of Qb and QaV exceeds Qa, generating an alarm event.

Item 59.

The method according to item 50, further comprising calculating a first fluid flow rate (Q1) at a first time instance and a second fluid flow rate (Q2) at a second time instance within a predetermined time interval (ΔT) based at least partly on the temporal signal profile, obtaining a reference measurement (Qref) at a third time instance within the same time interval (ΔT), and calculating an absolute average fluid flow rate (Qav) from said first (Q1) and second (Q2) fluid flow rates, the reference measurement (Qref), and the timing information of the first, second and third instances.

Item 60.

The method according to item 59, wherein the fluid flow rate (Q1) is a local minimum fluid flow rate (Qmin) and the second fluid flow rate (Q2) is a local maximum fluid flow rate (Qmax).

Item 61.

The method according to item 50, further comprising: defining an initial model (M₀); assigning the initial model (M₀) to a current model (CM); generating a parameter (P) that correlates with the fluid flow rate Q; acquiring flow calibration data (C); investigating whether a model validity criterion (MVC) is fulfilled or not by comparing parameter (P), calibration data (C) with the current model (CM), wherein in case the model validity criterion (MVC) is not fulfilled then repeatedly generating a new model (NM) and assigning the current model (CM) with new model (NM) until model validity criterion (MVC) is fulfilled; wherein in case the model validity criterion MVC is fulfilled, calculating a fluid flow rate (Q) based at least partly on the temporal signal profile.

Item 62.

The method according to item 61, further comprising one or more of acquiring blood pressure (BP) of the subject and comparing said blood pressure (BP) with the current model (CM); and storing of current model (CM) and available parameters (M, C, BP, P, TD, PD).

Item 63.

The method according to item 50, wherein the calculating of a fluid flow rate (Q) involves a pulse parameter P from one or more of amplitude, shape, and timing of the temporal signal profile.

Item 64.

The method according to any one of items 50-63, wherein the extracorporeal blood circuit (20) comprises a fluid pathway (10), a blood processing device (6), and at least one pumping device (3), and wherein the method further comprises detecting a pump pulse originating from the pumping device (3).

Further embodiments the method as set forth in items 50-64 may correspond to the embodiments of the device as set forth in items 4-36.

Item 100.

A computer-readable medium comprising computer instructions which, when executed by a processor, cause the processor to perform the method of any one of items 50-64.

Item 120.

A device for monitoring a fluid flow rate (Q) of a cardiovascular system of a subject, said device comprising: means for obtaining a time-dependent measurement signal (d(n)) from a pressure sensor (4a-4c) in an extracorporeal blood circuit (20) which is adapted for connection to the cardiovascular system, the pressure sensor (4a-4c) being arranged to detect a subject pulse originating from a subject pulse generator (3′) in the cardiovascular system of the subject, means for processing the measurement signal to obtain a pulse profile (e(n)) which is a temporal signal profile of the subject pulse, and means for calculating a fluid flow rate (Q) based at least partly on the temporal signal profile.

Embodiments of the device as set forth in item 120 may correspond to the embodiments of the device as set forth in items 2-38.

### APPENDIX A

### Brief Description of the Drawings

Exemplifying embodiments of the invention will now be described in more detail with reference to the accompanying schematic drawings.

FIG. A1 is a schematic view of a general fluid containing system in which the inventive data processing may be used for filtering a pressure signal.

FIG. A2 is a flow chart of a monitoring process according to an embodiment of the invention.

FIG. A3(a) is a plot of a pressure signal as a function of time, and FIG. A3(b) is a plot of the pressure signal after filtering.

FIG. A4 is a schematic view of a system for hemodialysis treatment including an extracorporeal blood flow circuit.

FIG. A5(a) is a plot in the time domain of a venous pressure signal containing both pump frequency components and a heart signal, and FIG. A5(b) is a plot of the corresponding signal in the frequency domain.

FIG. A6 is a plot of a predicted signal profile originating from a peristaltic pump in the system of FIG. A4.

FIG. A7 is a flow chart of a process for obtaining the predicted signal profile.

FIG. A8 is a plot to illustrate an extrapolation process for generating the predicted signal profile.

FIG. A9(a) is a plot to illustrate an interpolation process for generating the predicted signal profile, and FIG. A9(b) is an enlarged view of FIG. A9(a).

FIG. A10(a) represents a frequency spectrum of a pressure pulse originating from a pumping device at one flow rate, FIG. A10(b) represents corresponding frequency spectra for three different flow rates, wherein each frequency spectrum is given in logarithmic scale and mapped to harmonic numbers, FIG. A10(c) is a plot of the data in FIG. A10(b) in linear scale, and FIG. 10(d) is a phase angle spectrum corresponding to the frequency spectrum in FIG. A10(a).

FIG. A11 is schematic view of an adaptive filter structure operable to filter a measurement signal based on a predicted signal profile.

FIG. A12(a) illustrates a filtered pressure signal (top) and a corresponding heart signal (bottom), obtained from a venous pressure sensor, and FIG. A12(b) illustrates a filtered pressure signal (top) and a corresponding heart signal (bottom), obtained from an arterial pressure sensor.

### DETAILED DESCRIPTION OF EXEMPLIFYING EMBODIMENTS

In the following, exemplifying embodiments of the invention will be described with reference to fluid containing systems in general. Thereafter, the embodiments and implementations of the invention will be further exemplified in the context of systems for extracorporeal blood treatment.

Throughout the following description, like elements are designated by the same reference signs.

### General

FIG. A1 illustrates a fluid containing system in which a fluid connection C is established between a first fluid containing sub-system S1 and a second fluid containing sub-system S2. The fluid connection C may or may not transfer fluid from one sub-system to the other. A first pulse generator 3 is arranged to generate a series of pressure waves in the fluid within the first sub-system S1, and a second pulse generator 3′ is arranged to generate a series of pressure waves in the fluid within the second sub-system S2. A pressure sensor 4a is arranged to measure the fluid pressure in the first sub-system S1. Pressure waves generated by the second pulse generator 3′ will travel from the second sub-system S2 to the first sub-system S1, via the connection C, and thus second pulses originating from the second pulse generator 3′ will be detected by the pressure sensor 4a in addition to first pulses originating from the first pulse generator 3. It is to be noted that either one of the first and second pulse generators 3, 3′ may include more than one pulse-generating device. Further, any such pulse-generating device may or may not be part of the respective sub-system S1, S2.

The system of FIG. A1 further includes a surveillance device 25 which is connected to the pressure sensor 4a, and possibly to one or more additional pressure sensors 4b, 4c, as indicated in FIG. A1. Thereby, the surveillance device 25 acquires one or more pressure signals that are time-dependent to provide a real time representation of the fluid pressure in the first sub-system S1.

Generally, the surveillance device 25 is configured to monitor a functional state or functional parameter of the fluid containing system, by isolating and analysing one or more second pulses in one of the pressure signals. As will be further exemplified in the following, the functional state or parameter may be monitored to identify a fault condition, e.g. in the first or second sub-systems S1, S2, the second pulse generator 3′ or the fluid connection C. Upon identification of a fault condition, the surveillance device 25 may issue an alarm or warning signal and/or alert a control system of the first or second sub-systems S1, S2 to take appropriate action. Alternatively or additionally, the surveillance device 25 may be configured to record or output a time sequence of values of the functional state or parameter.

Depending on implementation, the surveillance device 25 may use digital components or analog components, or a combination thereof, for receiving and processing the pressure signal. The device 25 may thus be a computer, or a similar data processing device, with adequate hardware for acquiring and processing the pressure signal in accordance with different embodiments of the invention. Embodiments of the invention may e.g. be implemented by software instructions that are supplied on a computer-readable medium for execution by a processor 25a in conjunction with a memory unit 25b in the computer.

Typically, the surveillance device 25 is configured to continuously process the time-dependent pressure signal(s) to isolate any second pulses. This processing is schematically depicted in the flow chart of FIG. A2. The illustrated processing involves a step 201 of obtaining a first pulse profile u(n) which is a predicted temporal signal profile of the first pulse(s), and a step 202 of filtering the pressure signal d(n), or a pre-processed version thereof, in the time-domain, using the first pulse profile u(n), to essentially eliminate or cancel the first pulse(s) while retaining the second pulse(s) contained in d(n). In the context of the present disclosure, n indicates a sample number and is thus equivalent to a (relative) time point in a time-dependent signal. In step 203, the resulting filtered signal e(n) is then analysed for the purpose of monitoring the aforesaid functional state or parameter.

The first pulse profile is a shape template or standard signal profile, typically given as a time-sequence of data values, which reflects the shape of the first pulse in the time domain. The first pulse profile is also denoted “predicted signal profile” in the following description.

By “essentially eliminating” is meant that the first pulse(s) is(are) removed from the pressure signal to such an extent that the second pulse(s) can be detected and analysed for the purpose of monitoring the aforesaid functional state or parameter.

By filtering the pressure signal in the time-domain, using the first pulse profile, it is possible to essentially eliminate the first pulses and still retain the second pulses, even if the first and second pulses overlap or nearly overlap in the frequency domain. Such a frequency overlap is not unlikely, e.g. if one or both of the first and second pulses is made up of a combination of frequencies or frequency ranges.

Furthermore, the frequency, amplitude and phase content of the first pulse or the second pulse may vary over time. Such variations may be the result of an active control of the first and/or second pulse generator 3, 3′, or be caused by drifts in the first and/or second pulse generator 3, 3′ or by changes in the hydrodynamic properties of the sub-systems S1, S2 or the fluid connection C. Frequency variations may occur, e.g., when the second pulse generator 3′ is a human heart, and the second sub-system S2 thus is the blood system of a human. In healthy subjects under calm conditions, variations in heart rhythm (heart rate variability, HRV) may be as large as 15%. Unhealthy subjects may suffer from severe heart conditions such as atrial fibrillation and supraventricular ectopic beating, which may lead to an HRV in excess of 20%, and ventricular ectopic beating, for which HRV may be in excess of 60%. These heart conditions are not uncommon among, e.g., dialysis patients.

Any frequency overlap may make it impossible or at least difficult to isolate the second pulses in the pressure signal by conventional filtering in the frequency domain, e.g. by operating a comb filter and/or a combination of band-stop or notch filters, typically cascade coupled, on the pressure signal to block out all frequency components originating from the first pulse generator 3. Furthermore, frequency variations make it even harder to successfully isolate second pulses in the pressure signal, since the frequency overlap may vary over time. Even in the absence of any frequency overlap, frequency variations make it difficult to define filters in the frequency domain.

Depending on how well the first pulse profile represents the first pulse(s) in the pressure signal, it may be possible to isolate the second pulses by means of the inventive filtering in the time-domain even if the first and second pulses overlap in frequency, and even if the second pulses are much smaller in amplitude than the first pulses.

Still further, the inventive filtering in the time domain may allow for a faster isolation of second pulses in the pressure signal than a filtering process in the frequency domain. The former may have the ability to isolate a single second pulse in the pressure signal whereas the latter may need to operate on a sequence of first and second pulses in the pressure signal. Thus, the inventive filtering may enable faster determination of the functional state or functional parameter of the fluid containing system.

The effectiveness of the inventive filtering is exemplified in FIG. A3, in which FIG. A3(a) shows an example of a time-dependent pressure signal d(n) containing first and second pulses with a relative magnitude of 10:1. The first and second pulses have a frequency of 1 Hz and 1.33 Hz, respectively. Due to the difference in magnitude, the pressure signal is dominated by the first pulses. FIG. A3(b) shows the time-dependent filtered signal e(n) that is obtained after applying the inventive filtering technique to the pressure signal d(n). The filtered signal e(n) is made up of second pulses and noise. It should be noted that there is an absence of second pulses after about 4 seconds, which may be observed by the surveillance device (25 in FIG. A1) and identified as a fault condition of the fluid containing system.

Reverting to FIG. A2, the inventive data processing comprises two main steps: a determination of the first pulse profile u(n) (step 201) and a removal of one or more first pulses from a measurement signal d(n) using the first pulse profile u(n) (step 202).

There are many ways to implement these main steps. For example, the first pulse profile (standard signal profile) may be obtained in a reference measurement, based on a measurement signal from one or more of the pressure sensors 4a-4c in the first sub-system S1, suitably by identifying and possibly averaging a set of first pulse segments in the measurement signal(s). The first pulse profile may or may not be updated intermittently during the actual monitoring of the aforesaid functional state or parameter. Alternatively, a predetermined (i.e. predefined) standard signal profile may be used, which optionally may be modified according to a mathematical model accounting for wear in the first pulse generator, fluid flow rates, tubing dimensions, speed of sound in the fluid, etc. Further, the removal may involve subtracting the first pulse profile from the measurement signal at suitable amplitude and phase. The phase may be indicated by phase information which may be obtained from a signal generated by a phase sensor coupled to the first pulse generator 3, or from a control signal for the first pulse generator 3.

The inventive filtering may also be combined with other filtering techniques to further improve the quality of the filtered signal e(n). In one embodiment, the filtered signal e(n) could be passed through a bandpass filter with a passband in the relevant frequency range for the second pulses. If the second pulses originate from a human heart, the passband may be located within the approximate range of 0.5-4 Hz, corresponding to heart pulse rates of 30-240 beats per minute. In another embodiment, if the current frequency range (or ranges) of the second pulses is known, the passband of the bandpass filter could be actively controlled to a narrow range around the current frequency range. For example, such an active control may be applied whenever the rates of first and second pulses are found to differ by more than a certain limit, e.g. about 10%. The current frequency range may be obtained from the pressure signal, either by intermittently shutting off the first pulse generator 3, or intermittently preventing the first pulses from reaching the relevant pressure sensor 4a-4c. Alternatively, the current frequency range may be obtained from a dedicated sensor in either the first or the second sub-systems S1, S2, or based on a control unit (not shown) for the second pulse generator 3′. According to yet another alternative, the location and/or width of the passband could be set, at least in part, based on patient-specific information, i.e. existing data records for the patient, e.g. obtained in earlier treatments of the same patient. The patient-specific information may be stored in an internal memory of the surveillance device (25 in FIG. A1), on an external memory which is made accessible to the surveillance device, or on a patient card where the information is e.g. transmitted wirelessly to the surveillance device, e.g. by RFID (Radio Frequency IDentification).

These and other embodiments will be explained in further detail below, within the context of a system for extracorporeal blood treatment. To facilitate the following discussion, details of an exemplifying extracorporeal blood flow circuit will be first described.

### Monitoring in an Extracorporeal Blood Flow Circuit

FIG. A4 shows an example of an extracorporeal blood flow circuit 20 of the type which is used for dialysis. The extracorporeal blood flow circuit 20 (also denoted “extracorporeal circuit”) comprises components 1-14 to be described in the following. Thus, the extracorporeal circuit 20 comprises an access device for blood extraction in the form of an arterial needle 1, and an arterial tube segment 2 which connects the arterial needle 1 to a blood pump 3 which may be of peristaltic type, as indicated in FIG. A4. At the inlet of the pump there is a pressure sensor 4b (hereafter referred to as “arterial sensor”) which measures the pressure before the pump in the arterial tube segment 2. The blood pump 3 forces the blood, via a tube segment 5, to the blood-side of a dialyser 6. Many dialysis machines are additionally provided with a pressure sensor 4c (hereafter referred to as “system sensor”) that measures the pressure between the blood pump 3 and the dialyser 6. The blood is lead via a tube segment 10 from the blood-side of the dialyser 6 to a venous drip chamber or deaeration chamber 11 and from there back to the patient via a venous tube segment 12 and an access device for blood reintroduction in the form of a venous needle 14. A pressure sensor 4a (hereafter referred to as “venous sensor”) is provided to measure the pressure on the venous side of the dialyser 6. In the illustrated example, the pressure sensor 4a measures the pressure in the venous drip chamber. Both the arterial needle 1 and the venous needle 14 are connected to the patient by means of a blood vessel access. The blood vessel access may be of any suitable type, e.g. a fistula, a Scribner-shunt, a graft, etc. Depending on the type of blood vessel access, other types of access devices may be used instead of needles, e.g. catheters. The access devices 1, 14 may alternatively be combined into a single unit.

In relation to the fluid containing system in FIG. A1, the extracorporeal circuit 20 corresponds to the first sub-system S1, the blood pump 3 (as well as any further pulse source(s) within or associated with the extracorporeal circuit 20, such as a dialysis solution pump, valves, etc) corresponds to the first pulse generator 3, the blood system of the patient corresponds to the second sub-system S2, and the fluid connection C corresponds to at least one of the venous-side and arterial-side fluid connections between the patient and the extracorporeal circuit 20.

In FIG. A4, a control unit 23 is provided, i.a., to control the blood flow in the extracorporeal circuit 20 by controlling the revolution speed of the blood pump 3. The extracorporeal circuit 20 and the control unit 23 may form part of an apparatus for extracorporeal blood treatment, such as a dialysis machine. Although not shown or discussed further it is to be understood that such an apparatus performs many other functions, e.g. controlling the flow of dialysis fluid, controlling the temperature and composition of the dialysis fluid, etc.

The system in FIG. A4 also includes a surveillance/monitoring device 25, which is connected to receive a pressure signal from at least one of the pressure sensors 4a-4c and which executes the inventive data processing. In the example of FIG. A4, the surveillance device 25 is also connected to the control unit 23. Alternatively or additionally, the device 25 may be connected to a pump sensor 26 for indicating the revolution speed and/or phase of the blood pump 3. It is to be understood that the surveillance device 25 may include inputs for further data, e.g. any other system parameters that represent the overall system state (see e.g. discussion with reference to FIG. A7 below). The device 25 is tethered or wirelessly connected to a local or remote device 27 for generating an audible/visual/tactile alarm or warning signal. Alternatively or additionally, either device 25, 27 may include a display or monitor for displaying the functional state or parameter resulting from the analysis step (203 in FIG. A2), and/or the filtered signal e(n) resulting from the filtering step (202 in FIG. A2), e.g. for visual inspection.

In FIG. A4, the surveillance device 25 comprises a data acquisition part 28 for pre-processing the incoming signal(s), e.g. including an A/D converter with a required minimum sampling rate and resolution, one or more signal amplifiers, and one or more filters to remove undesired components of the incoming signal(s), such as offset, high frequency noise and supply voltage disturbances.

After the pre-processing in the data acquisition part 28, the pre-processed pressure signal is provided as input to a main data processing part 29, which executes the inventive data processing. FIG. A5(a) shows an example of such a pre-processed pressure signal in the time domain, and FIG. A5(b) shows the corresponding power spectrum, i.e. the pre-processed pressure signal in the frequency domain. The power spectrum reveals that the detected pressure signal contains a number of different frequency components emanating from the blood pump 3. In the illustrated example, there is a frequency component at the base frequency (f₀) of the blood pump (at 1.5 Hz in this example), as well as its harmonics 2f₀, 3f₀ and 4f₀. The base frequency, also denoted pump frequency in the following, is the frequency of the pump strokes that generate pressure waves in the extracorporeal circuit 20. For example, in a peristaltic pump of the type shown in FIG. A4, two pump strokes are generated for each full revolution of the rotor 3a. FIG. A5(b) also indicates the presence of a frequency component at half the pump frequency (0.5f₀) and harmonics thereof, in this example at least f₀, 1.5f₀, 2f₀ and 2.5f₀. FIG. A5(b) also shows a heart signal (at 1.1 Hz) which in this example is approximately 40 times weaker than the blood pump signal at the base frequency f₀.

The main data processing part 29 executes the aforesaid steps 201-203. In step 202, the main data processing part 29 operates to filter the pre-processed pressure signal in the time domain, and outputs a filtered signal or monitoring signal (e(n) in FIG. A2) in which the signal components of the blood pump 3 have been removed. The monitoring signal still contains any signal components that originate from the patient (cf. FIG. A3(b)), such as pressure pulses caused by the beating of the patient's heart. There are a number of sources to cyclic physiological phenomena that may generate pressure pulses in the blood stream of the patient, including the heart, the breathing system, or the vasomotor, which is controlled by the autonomic nervous system. Thus, the monitoring signal may contain pressure pulses resulting from a combination of cyclic phenomena in the patient. Generally speaking, the signal components in the monitoring signal may originate from any type of physiological phenomenon in the patient, or combinations thereof, be it cyclic or non-cyclic, repetitive or non-repetitive, autonomous or non-autonomous.

Depending on implementation, the surveillance device 25 may be configured apply further filtering to the monitoring signal to isolate signal components originating from a single cyclic phenomenon in the patient. Alternatively, such signal component filtering is done during the pre-processing of the pressure signal (by the data acquisition part 28). The signal component filtering may be done in the frequency domain, e.g. by applying a cut-off or bandpass filter, since the signal components of the different cyclic phenomena in the patient are typically separated in the frequency domain. Generally, the heart frequency is about 0.5-4 Hz, the breathing frequency is about 0.15-0.4 Hz, the frequency of the autonomous system for regulation of blood pressure is about 0.04-0.14 Hz, the frequency of the autonomous system for regulation of body temperature is about 0.04 Hz.

The surveillance device 25 could be configured to monitor the breathing pattern of the patient, by identifying breathing pulses in the monitoring signal. The resulting information could be used for on-line surveillance for apnoea, hyperventilation, hypoventilation, asthmatic attacks or other irregular breathing behaviours of the patient. The resulting information could also be used to identify coughing, sneezing, vomiting or seizures. The vibrations resulting from coughing/sneezing/vomiting/seizures might disturb other measurement or surveillance equipment that is connected to the patient or the extracorporeal circuit 20. The surveillance device 25 may be arranged to output information about the timing of any coughing/sneezing/vomiting/seizures, such that other measurement or surveillance equipment can take adequate measures to reduce the likelihood that the coughing/sneezing/vomiting/seizures results in erroneous measurements or false alarms. Of course, the ability of identifying coughing/sneezing/vomiting/seizures may also have a medical interest of its own.

The surveillance device 25 could be configured to monitor the heart rate of the patient, by identifying heart pulses in the monitoring signal.

The surveillance device 25 could be configured to collect and store data on the time evolution of the heart rate, the breathing pattern, etc, e.g. for subsequent trending or statistical analysis.

The surveillance device 25 may be configured to monitor the integrity of the fluid connection between the patient and the extracorporeal circuit 20, in particular the venous-side fluid connection (via access device 14). This could be done by monitoring the presence of a signal component originating from, e.g., the patient's heart or breathing system in the monitoring signal. Absence of such a signal component may be taken as an indication of a failure in the integrity of the fluid connection C, and could bring the device 25 to activate an alarm and/or stop the blood flow, e.g. by stopping the blood pump 3 and activating a clamping device 13 on the tube segment 12. For monitoring the integrity of the venous-side fluid connection, also known as VNM (Venous Needle Monitoring), the surveillance device 25 may be configured to generate the monitoring signal based on a pressure signal from the venous sensor 4a. The device 25 may also be connected to pressure sensors 4b, 4c, as well as any additional pressure sensors included in the extracorporeal circuit 20.

The extracorporeal circuit 20 may have the option to operate in a hemodiafiltration mode (HDF mode), in which the control unit 23 activates a second pumping device (HDF pump, not shown) to supply an infusion solution into the blood line upstream and/or downstream of the dialyser 6, e.g. into one or more of tube segments 2, 5, 10 or 12.

### Obtaining the Predicted Signal Profile of First Pulses

This section describes different embodiments for predicting or estimating the signal profile of first pulses in the system shown in FIG. A4. The predicted signal profile is typically given as a series of pressure values over a period of time normally corresponding to at least one complete pump cycle of the blood pump 3.

FIG. A6 illustrates an example of a predicted signal profile for the system in FIG. A4. Since the blood pump 3 is a peristaltic pump, in which two rollers 3b engage a tube segment during a full revolution of the rotor 3a, the pressure profile consists of two pump strokes. The pump strokes may result in different pressure values (pressure profiles), e.g. due to slight differences in the engagement between the rollers 3b and the tube segment, and thus it may be desirable for the predicted signal profile to represent both pump strokes. If a lower accuracy of the predicted signal profile can be tolerated, i.e. if the output of the subsequent removal process is acceptable, the predicted signal profile might represent one pump stroke only.

On a general level, the predicted signal profile may be obtained in a reference measurement, through mathematical simulation of the fluid system, or combinations thereof.

### Reference Measurement

A first main group of methods for obtaining the predicted signal profile is based on deriving a time-dependent reference pressure signal (“reference signal”) from a pressure sensor in the system, typically (but not necessarily) from the same pressure sensor that provides the measurement signal (pressure signal) that is to be processed for removal of first pulses. During this reference measurement, the second pulses are prevented from reaching the relevant pressure sensor, either by shutting down/deactivating the second pulse generator 3′ or by isolating the pressure sensor from the second pulses. In the system of FIG. A4, the reference measurement could be carried out during a priming phase, in which the extracorporeal circuit 20 is detached from the patient and a priming fluid is pumped through the blood lines. Alternatively, the reference measurement could be carried in a simulated treatment with blood or any other fluid. Optionally, the reference measurement could involve averaging a plurality of pressure profiles to reduce noise. For example, a plurality of relevant signal segments may be identified in the reference signal, whereupon these segments are aligned to achieve a proper overlap of the pressure profiles in the different segments and then added together. The identifying of relevant signal segments may be at least partially based on timing information which indicates the expected position of each first pulse in the reference signal. The timing information may be obtained from a trigger point in the output signal of the pump sensor 26, in a control signal of the control unit 23, or in the pressure signal from another one of the pressure sensors 4a-4c. For example, a predicted time point of a first pulse in the reference signal can be calculated based on a known difference in arrival time between the trigger point and the pressure sensor that generates the reference signal. In variant, if the reference signal is periodic, relevant signal segments may be identified by identifying crossing points of the reference signal with a given signal level, wherein the relevant signal segments are identified to extend between any respective pairs of crossing points.

In a first embodiment, the predicted signal profile is directly obtained in a reference measurement before the extracorporeal circuit 20 is connected to the patient, and is then used as input to the subsequent removal process, which is executed when the extracorporeal circuit 20 is connected to the patient. In this embodiment, it is thus assumed that the predicted signal profile is representative of the first pulses when the system is connected to the patient. Suitably, the same pump frequency/speed is used during the reference measurement and during the removal process. It is also desirable that other relevant system parameters are maintained essentially constant.

FIG. A7 is a flow chart of a second embodiment. In the second embodiment, a reference library or database is first created based on the reference measurement (step 701). The resulting reference library is typically stored in a memory unit, e.g. RAM, ROM, EPROM, HDD, Flash, etc (cf. 25b in FIG. A1) of the surveillance device (cf. 25 in FIG. A1). During the reference measurement, reference pressure signals are acquired for a number of different operational states of the extracorporeal circuit. Each operational state is represented by a unique combination of system parameter values. For each operational state, a reference profile is generated to represent the signal profile of the first pulses. The reference profiles together with associated system parameter values are then stored in the reference library, which is implemented as a searchable data structure, such as a list, look-up table, search tree, etc.

During the actual monitoring process, i.e. when first pulses are to be eliminated from the measurement signal, current state information indicating the current operational state of the fluid containing system is obtained from the system, e.g. from a sensor, a control unit or otherwise (step 702). The current state information may include a current value of one or more system parameters. The current value is then matched against the system parameter values in the reference library. Based on the matching, one or more reference profiles are selected (step 703) and used for preparing the predicted signal profile (step 704).

Generally, the aforesaid system parameters represent the overall system state, including but not limited to the structure, settings, status and variables of the fluid containing system or its components. In the system of FIG. A4, exemplary system parameters may include:

Pump-Related Parameters:
number of active pumps connected directly or indirectly (e.g. in a fluid preparation system for the dialyser) to the extracorporeal circuit, type of pumps used (roller pump, membrane pump, etc), flow rate, revolution speed of pumps, shaft position of pump actuator (e.g. angular or linear position), etc

Dialysis Machine Settings:
temperature, ultrafiltration rate, mode changes, valve position/changes, etc

Disposable Dialysis Equipment/Material:
information on pump chamber/pump segment (material, geometry and wear status), type of blood line (material and geometry), type of dialyser, type and geometry of access devices, etc

Dialysis System Variables:
actual absolute pressures of the system upstream and downstream of the blood pump, e.g. venous pressure (from sensor 4a), arterial pressure (from sensor 4b) and system pressure (from sensor 4c), gas volumes trapped in the flow path, blood line suspension, fluid type (e.g. blood or dialysis fluid), etc

Patient Status:
blood access properties, blood properties such as e.g. hematocrit, plasma protein concentration, etc

It is to be understood that any number or combination of system parameters may be stored in the reference library and/or used as search variables in the reference library during the monitoring process.

In the following, the second embodiment will be further explained in relation to a number of examples. In all of these examples, the pump revolution frequency (“pump frequency”), or a related parameter (e.g. blood flow rate) is used to indicate the current operational state of the fluid containing system during the monitoring process. In other words, the pump frequency is used as search variable in the reference library. The pump frequency may e.g. be given by a set value for the blood flow rate output from the control unit, or by an output signal of a sensor that indicates the frequency of the pump (cf. pump sensor 26 in FIG. A4). Alternatively, the pump frequency could be obtained by frequency analysis of the pressure signal from any of the sensors 4a-4c during operation of the fluid system. Such frequency analysis could be achieved by applying any form of harmonics analysis to the pressure signal, such as Fourier or wavelet analysis. As indicated in FIG. A5(b), the base frequency f₀ of the pump can be identified in a resulting power spectrum.

In a first example, the reference library is searched for retrieval of the reference profile that is associated with the pump frequency that lies closest to the current pump frequency. If no exact match is found to the current pump frequency, an extrapolation process is executed to generate the predicted signal profile. In the extrapolation process, the retrieved reference profile is scaled in time to the current pump cycle, based on the known difference (“pump frequency difference”) between the current pump frequency and the pump frequency associated with the retrieved reference profile. The amplitude scale may also be adjusted to compensate for amplitude changes due to pump frequency, e.g. based on a known function of amplitude as a function of pump frequency. FIG. A8 illustrates a reference profile r₁(n) obtained at a flow rate of 470 ml/min, and predicted signal profile u(n) which is obtained by scaling the reference profile to a flow rate of 480 ml/min. For comparison only, a reference profile r_{actual}(n) obtained at 480 ml/min is also shown, to illustrate that extrapolation process indeed may yield a properly predicted signal profile.

In a second example, the reference library is again searched based on current pump frequency. If no exact match is found to the current pump frequency, a combination process is executed to generate the predicted signal profile. Here, the reference profiles associated with the two closest matching pump frequencies are retrieved and combined. The combination may be done by re-scaling the pump cycle time of the retrieved reference profiles to the current pump frequency and by calculating the predicted signal profile via interpolation of the re-scaled reference profiles. For example, the predicted signal profile u(n) at the current pump frequency v may be given by:

u(n)=g(v−vᵢ)·rᵢ(n)+(1−g(v−vᵢ))·rⱼ(n),

wherein rᵢ(n) and rⱼ(n) denotes the two retrieved reference profiles, obtained at a pump frequency vᵢ and vⱼ, respectively, after re-scaling to the current pump frequency v, and g is a relaxation parameter which is given as a function of the frequency difference (v−vᵢ), wherein vᵢ≦v≦vⱼ and 0≦g≦1. The skilled person realizes that the predicted signal profile u(n) may be generated by combining more than two reference profiles.

FIG. A9(a) illustrates a predicted signal profile u(n) at a current flow rate of 320 ml/min for a measurement signal obtained from the venous sensor 4a in the system of FIG. A4. The predicted signal profile u(n) has been calculated as an average of a reference profile r₁(n) obtained at a flow rate of 300 ml/min from the venous sensor and a reference profile r₂(n) obtained at a flow rate of 340 ml/min from the venous sensor. For comparison only, a reference profile r_{actual}(n) obtained at 320 ml/min is also shown, to illustrate that the combination process indeed may yield a properly predicted signal profile. In fact, the differences are so small that they are only barely visible in the enlarged view of FIG. A9(b).

The first and second examples may be combined, e.g. by executing the extrapolation process of the first example if the pump frequency difference is less than a certain limit, and otherwise executing the combination process of the second example.

In a third embodiment, like in the second embodiment shown in FIG. A7, a number of reference signals are acquired in the reference measurement, wherein each reference signal is obtained for a specific combination of system parameter values. The reference signals are then processed for generation of reference spectra, which are indicative of the energy and phase angle as function of frequency. These reference spectra may e.g. be obtained by Fourier analysis, or equivalent, of the reference signals. Corresponding energy and phase data are then stored in a reference library together with the associated system parameter values (cf. step 701 in FIG. A7). The implementation of the reference library may be the same as in the second embodiment.

During the actual monitoring process, i.e. when first pulses are to be eliminated from the measurement signal, a current value of one or more system parameters is obtained from the fluid containing system (cf. step 702 in FIG. A7). The current value is then matched against the system parameter values in the reference library. Based on the matching, a specific set of energy and phase data may be retrieved from the reference library to be used for generating the predicted signal profile (cf. step 703 in FIG. A7). Generally, the predicted signal profile is generated by adding sinusoids of appropriate frequency, amplitude and phase, according to the retrieved energy and phase data (cf. step 704 in FIG. A7).

Generally speaking, without limiting the present disclosure, it may be advantageous to generate the predicted signal profile from energy and phase data when the first pulses (to be removed) contain only one or a few base frequencies (and harmonics thereof), since the predicted signal profile can be represented by a small data set (containing energy and phase data for the base frequencies and the harmonics). One the other hand, when the power spectrum of the first pulses is more complex, e.g. a mixture of many base frequencies, it may instead be preferable to generate the predicted signal profile from one or more reference profiles.

FIG. A10(a) represents an energy spectrum of a reference signal acquired at a flow rate of 300 ml/min in the system of FIG. A4. In this example, the reference signal essentially consists of a basic pump frequency at 1.2 Hz (f₀, first harmonic) and a set of overtones of this frequency (second and further harmonics). Compared to the power spectrum of FIG. A5(b), the pressure signals used for generating the graphs in FIG. A10(a)-10(d) do not contain any significant frequency component at 0.5f₀ and its harmonics. The graph in FIG. A10(a) displays the relative energy distribution, wherein the energy values have been normalized to the total energy for frequencies in the range of 0-10 Hz. FIG. A10(b) represents energy spectra of reference signals acquired at three different flow rates in the system of FIG. A4. The energy spectra are given in logarithmic scale versus harmonic number (first, second, etc). As shown, an approximate linear relationship can be identified between the logarithmic energy and harmonic number for the first four to five harmonic numbers. This indicates that each energy spectrum may be represented by a respective exponential function. FIG. A10(c) illustrates the data of FIG. A10(b) in linear scale, wherein a respective polynomial function has been fitted to the data. As indicated in FIGS. A10(a)-A10(c), the energy spectra may be represented in different formats in the reference library, e.g. as a set of energy values associated with discrete frequency values or harmonic numbers, or as an energy function representing energy versus frequency/harmonic number.

FIG. A10(d) illustrates a phase angle spectrum acquired together with the energy spectrum in FIG. A10(a), i.e. for a flow rate of 300 ml/min. The graph in FIG. A10(d) illustrates phase angle as a function of frequency, and a linear function has been fitted to the data. In an alternative representation (not shown), the phase spectrum may be given as a function of harmonic number. Like the energy spectra, the phase spectra may be represented in different formats in the reference library, e.g. as a set of phase angle values associated with discrete frequency values or harmonic numbers, or as a phase function representing phase angle versus frequency/harmonic number.

From the above, it should be understood that the energy and phase data that are stored the reference library can be used to generate the predicted signal profile. Each energy value in the energy data corresponds to an amplitude of a sinusoid with a given frequency (the frequency associated with the energy value), wherein the phase value for the given frequency indicates the proper phase angle of the sinousoid. This method of preparing the predicted signal profile by combining (typically adding) sinusoids of appropriate frequency, amplitude and phase angle allows the predicted signal profile to include all harmonics of the pump frequency within a desired frequency range.

When a predicted signal profile is to be generated, the reference library is first searched based on a current value of one or more system parameters, such as the current pump frequency. If no exact match is found in the reference library, a combination process may be executed to generate the predicted signal profile. For example, the two closest matching pump frequencies may be identified in the reference library and the associated energy and phase data may be retrieved and combined to form the predicted signal profile. The combination may be done by interpolating the energy data and the phase data. In the example of FIGS. A10(a)-A10(d), an interpolated energy value may be calculated for each harmonic number, and similarly an interpolated phase value could be calculated for each harmonic number. Any type of interpolation function could be used, be it linear or non-linear.

In the first, second and third embodiments, the reference signals and the measurement signals are suitably obtained from the same pressure sensor unit in the fluid containing system. Alternatively, different pressure sensor units could be used, provided that the pressure sensor units yield identical signal responses with respect to the first pulses or that the signal responses can be matched using a known mathematical relationship.

To further improve the first, second and third embodiments, the process of generating the predicted signal profile may also involve compensating for other potentially relevant factors that differ between the reference measurement and the current operational state. These so-called confounding factors may comprise one or more of the system parameters listed above, such as absolute average venous and arterial pressures, temperature, blood hematocrit/viscosity, gas volumes, etc. This compensation may be done with the use of predefined compensation formulas or look-up tables.

In further variations, the second and third embodiments may be combined, e.g. in that the reference library stores not only energy and phase data, but also reference profiles, in association with system parameter value(s). When an exact match is found in the library, the reference profile is retrieved from the library and used as the predicted signal profile, otherwise the predicted signal profile is obtained by retrieving and combining (e.g. interpolating) the energy and phase data, as in the third embodiment. In a variant, the predicted signal profile u(n) at the current pump frequency v is obtained by:

u(n)=rᵢ(n)−r^{f}ᵢ(n)+r^{f}(n),

wherein rᵢ(n) denotes a reference profile that is associated with the closest matching pump frequency vᵢ in the reference library, r^{f}ᵢ(n) denotes a reference profile that is reconstructed from the energy and phase data associated with the closest matching pump frequency vᵢ in the reference library, and r^{f}(n) denotes an estimated reference profile at the current pump frequency v. The estimated reference profile r^{f}(n) may be obtained by applying predetermined functions to estimate the energy and phase data, respectively, at the current pump frequency v based on the energy and phase data associated with the closest matching pump frequency vᵢ. With reference to FIGS. A10(b)-A10(c), such a predetermined function may thus represent the change in energy data between different flow rates. Alternatively, the estimated reference profile r^{f}(n) may be obtained by retrieving and combining (e.g. interpolating) energy and phase data for the two closest matching pump frequencies vᵢ and vⱼ as in the third embodiment.

In a further variant, the reference measurement is made during regular operation of the fluid containing system, instead of or in addition to any reference measurements made before regular operation (e.g. during priming or simulated treatments with blood). Such a variant presumes that it is possible to intermittently shut off the second pulse generator, or to intermittently prevent the second pulses from reaching the relevant pressure sensor. This approach is more difficult in the extracorporeal circuit 20 of FIG. A4 if the reference signals and the measurement signals are obtained from the one and the same pressure sensor. However, this approach can e.g. be applied if the fluid system includes one pressure sensor that is substantially isolated from the second pulses. In such a situation, the reference profile (or reference spectra) may be obtained from the isolated sensor, and used for generating the predicted signal profile (optionally after adjustment/modification for differences in confounding factors), which is then used for removing first pulses from a measurement signal that contains both first and second pulses. For example, the pressure signal from the system sensor 4c in the circuit 20 of FIG. A4 may be essentially isolated from the second pulses that originate from the patient, and this pressure signal may thus be used in a reference measurement.

As explained above, the extracorporeal circuit 20 in FIG. A4 may be switched into a HDF mode, in which an additional HDF pump is activated to supply an infusion liquid into the blood line of the extracorporeal circuit 20. Such a change of operating mode may cause a change in the signal characteristics of the first pulses in the measurement signal. Thus, it may necessary to account for this change, by ensuring that the reference library includes appropriate reference data (reference profiles and/or energy and phase angle data) associated with this operational state.

Alternatively, it may be desirable to isolate the pressure pulses originating from the HDF pump. This could be achieved by obtaining a reference profile from the pressure signal of the arterial sensor 4b (FIG. A4). The arterial pressure signal includes pressure pulses originating from the patient and from the blood pump 3, whereas pressure pulses originating from the HDF pump are significantly damped by the patient and the blood pump 3, respectively, and thus barely reach the arterial sensor 4b. On the other hand, the pressure signals of the venous sensor 4a and the system sensor 4c contain pressure pulses originating from both the patient, the blood pump 3 and the HDF pump. Thus, the arterial pressure signal may be used for obtaining the predicted signal profile of the combined pressure pulses originating from the blood pump 3 and the patient as they should look in the pressure signal from the venous sensor 4a or the system sensor 4c. The predicted signal profile may then be used for isolating the pressure pulses originating from the HDF pump in the pressure signal from the venous sensor 4a or the system sensor 4c. In this example, the patient and the extracorporeal circuit 20 could be regarded as a first sub-system (S1 in FIG. A1) and the HDF pump and the associated infusion tubing could be regarded as a second sub-system (S2 in FIG. A1), which are connected via a fluid connection. Thus, in this example, the inventive data processing is not applied to isolate pulses originating from a cyclic physiological phenomenon in the patient, but pulses originating from another pump in the fluid system. It should be realized that in other arrangements, the reference profile may be obtained from the pressure signal of the venous sensor 4a (FIG. A4), and used for processing the pressure signal of the arterial sensor 4b or system sensor 4c.

### Simulations

As an alternative to the use of reference measurements, the predicted signal profile may be obtained directly through simulations, i.e. calculations using a mathematical model of the fluid containing system, based on current state information indicating the current operational state of the system. Such current state information may include a current value of one or more of the above-mentioned system parameters. The model may be based on known physical relationships of the system components (or via an equivalent representation, e.g. by representing the system as an electrical circuit with fluid flow and pressure being given by electrical current and voltage, respectively). The model may be expressed, implicitly or explicitly, in analytical terms. Alternatively, a numerical model may be used. The model could be anything from a complete physical description of the system to a simple function. In one example, such a simple function could convert data on the instantaneous angular velocity of the pump rotor 3a to a predicted signal profile, using empirical or theoretical data. Such data on the instantaneous angular velocity might be obtained from the pump sensor 26 in FIG. A4.

In another embodiment, simulations are used to generate reference profiles for different operational states of the system. These reference profiles may then be stored in a reference library, which may be accessed and used in the same way as described above for the second and third embodiments. It is also to be understood that reference profiles (and/or corresponding energy and phase angle data) obtained by simulations may be stored together with reference profiles (and/or corresponding energy and phase angle data) obtained by reference measurement.

### Removal of First Pulses

There are several different ways of removing one or more first pulses from the measurement signal, using the predicted signal profile. Here, two different removal processes will be described: Single Subtraction and Adaptive Filtering. Of course, the description of removal processes and their implementations is not comprehensive (neither of the different alternatives nor of the implementations), which is obvious to a person skilled in the art.

Depending on implementation, the predicted signal profile may be input to the removal process as is, or the predicted signal profile may be duplicated to construct an input signal of suitable length for the removal process.

### Single Subtraction

In this removal process, a single predicted signal profile is subtracted from the measurement signal. The predicted signal profile may be shifted and scaled in time and scaled in amplitude in any way, e.g. to minimize the error of the removal. Different minimization criterions may be used for such an auto-scaling, e.g., minimizing the sum of the squared errors, or the sum of the absolute errors. Alternatively or additionally, the predicted signal profile is shifted in time based on timing information that indicates the expected timing of the first pulse(s) in the measurement signal. The timing information may be obtained in the same way as described above in relation to the averaging of pressure segments in the reference signal.

One potential limitation of this removal process is that the relationship between different frequencies in the predicted signal profile is always the same, since the process only shifts and scales the predicted signal profile. Thus, it is not possible to change the relationship between different harmonic frequencies, neither is it possible to use only some of the frequency content in the predicted signal profile and to suppress other frequencies. To overcome this limitation, adaptive filtering may be used since it uses a linear filter before subtraction, e.g. as described in the following.

### Adaptive Filtering

FIG. A11 is a schematic overview of an adaptive filter 30 and an adaptive filter structure which is designed to receive the predicted signal profile u(n) and a measurement signal d(n), and to output an error signal e(n) which forms the aforesaid monitoring signal in which the first pulses are removed.

Adaptive filters are well-known electronic filters (digital or analog) that self-adjust their transfer function according to an optimizing algorithm. Specifically, the adaptive filter 30 includes a variable filter 32, typically a finite impulse response (FIR) filter of length M with filter coefficients w(n).

Even if adaptive filters are known in the art, they are not readily applicable to cancel the first pulses in the measurement signal d(n). In the illustrated embodiment, this has been achieved by inputting the predicted signal profile u(n) to the variable filter 32, which processes the predicted signal profile u(n) to generate an estimated measurement signal \hat{d}(n), and to an adaptive update algorithm 34, which calculates the filter coefficients of the variable filter 32 based on the predicted signal profile u(n) and the error signal e(n). The error signal e(n) is given by the difference between the measurement signal d(n) and the estimated measurement signal \hat{d}(n).

Basically, the adaptive filtering also involves a subtraction of the predicted signal profile u(n) from the measurement signal d(n), since each of the filter coefficients operates to shift and possibly re-scale the amplitude of the predicted signal profile u(n). The estimated measurement signal \hat{d}(n), which is subtracted from the measurement signal d(n) to generate the error signal e(n), is thus formed as a linear combination of M shifted predicted signal profiles u(n), i.e. a linear filtering of u(n).

The adaptive update algorithm 34 may be implemented in many different ways, some of which will be described below. The disclosure is in no way limited to these examples, and the skilled person should have no difficulty of finding further alternatives based on the following description.

There are two main approaches to adaptive filtering: stochastic and deterministic. The difference lies in the minimization of the error signal e(n) by the update algorithm 34, where different minimization criteria are obtained whether e(n) is assumed to be stochastic or deterministic. A stochastic approach typically uses a cost function J with an expectation in the minimization criterion, while a deterministic approach typically uses a mean. The squared error signal e²(n) is typically used in a cost function when minimizing e(n), since this results in one global minimum. In some situations, the absolute error |e(n)| may be used in the minimization, as well as different forms of constrained minimizations. Of course, any form of the error signal may be used, however convergence towards a global minimum is not always guaranteed and the minimization may not always be solvable.

In a stochastic description of the signal, the cost function may typically be according to,

J(n)=E{|e(n)|²},

and in a deterministic description of the signal the cost function may typically be according to,

J(n)=Σe²(n).

The first pulses will be removed from the measurement signal d(n) when the error signal e(n) (cost function J(n)) is minimized. Thus, the error signal e(n) will be cleaned from first pulses while retaining the second pulses, once the adaptive filter 30 has converged and reached the minimum error.

In order to obtain the optimal filter coefficients w(n) for the variable filter 32, the cost function J needs to be minimized with respect to the filter coefficients w(n). This may be achieved with the cost function gradient vector ∇J, which is the derivative of J with respect to the different filter coefficients w₀, w₁, . . . , w_{M-1}. Steepest Descent is a recursive method (not an adaptive filter) for obtaining the optimal filter coefficients that minimize the cost function J. The recursive method is started by giving the filter coefficients an initial value, which is often set to zero, i.e., w(0)=0. The filter coefficients is then updated according to,

w(n+1)=w(n)+½μ[−∇J(n)],

where w is given by,

w=[w₀ w₁ . . . w_{M-1}]^{T} M×1.

Furthermore, the gradient vector ∇J points in the direction in which the cost is growing the fastest. Thus, the filter coefficients are corrected in the direction opposite to the gradient, where the length of the correction is influenced through the step size parameter μ. There is always a risk for the Steepest Descent algorithm to diverge, since the algorithm contains a feedback. This sets boundaries on the step size parameter μ in order to ensure convergence. It may be shown that the stability criterion for the Steepest Descent algorithm is given by,

$$0 < μ < \frac{2}{{λ}_{max}}$$

where λₘₐₓ is the largest eigenvalue of R, the correlation matrix of the predicted signal profile u(n), given by

$$R = E [ \overline{u} ( n ) {\overline{u}}^{T} ( n ) ] = [ \begin{matrix}r ( 0 ) & r ( 1 ) & … & r ( M - 1 ) \\ r ( 1 ) & r ( 0 ) & & r ( M - 2 ) \\ ⋮ & ⋮ & ⋱ & ⋮ \\ r ( M - 1 ) & r ( M - 2 ) & … & r ( 0 )\end{matrix} ] ,$$

where ū(n) is given by,

ū(n)=[u(n)u(n−1) . . . u(n−M+1)]^{T} M×1.

If the mean squared error (MSE) cost function (defined by J=E{|e(n)|²}) is used, it may be shown that the filter coefficients are updated according to,

w(n+1)=w(n)+μE[ū(n)e(n)],

where e(n) is given by,

e(n)=d(n)−ū^{T}(n)w(n).

The Steepest Descent algorithm is a recursive algorithm for calculation of the optimal filter coefficients when the statistics of the signals are known. However, this information is often unknown. The Least Mean Squares (LMS) algorithm is a method that is based on the same principles as the Steepest Descent algorithm, but where the statistics is estimated continuously. Thus, the LMS algorithm is an adaptive filter, since the algorithm can adapt to changes in the signal statistics (due to continuous statistic estimations), although the gradient may become noisy. Because of the noise in the gradient, the LMS algorithm is unlikely to reach the minimum error Jₘᵢₙ, which the Steepest Descent algorithm does. Instantaneous estimates of the expectation are used in the LMS algorithm, i.e., the expectation is removed. Thus, for the LMS algorithm, the update equation of the filter coefficients becomes

w(n+1)=w(n)+μū(n)e(n).

The convergence criterion of the LMS algorithm is the same as for the Steepest Descent algorithm. In the LMS algorithm, the step size is proportional to the predicted signal profile u(n), i.e., the gradient noise is amplified when the predicted signal profile is strong. One solution to this problem is to normalize the update of the filter coefficients with

∥ū(n)∥²=ū^{T}(n) u̅(n).

The new update equation of the filter coefficients is called the Normalized LMS, and is given by

$$w ( n + 1 ) = w ( n ) + \frac{\tilde{μ}}{a + {‖ \overline{u} ( n ) ‖}^{2}} \overline{u} ( n ) e ( n ) ,$$

where 0<\tilde{μ}<2, and a is a positive protection constant.

There are many more different alternatives to the LMS algorithm, where the step size is modified. One of them is to use a variable adaptation step,

w(n+1)=w(n)+α(n) u̅(n)e(n),

where α(n) for example may be,

$$α ( n ) = \frac{1}{n + c} ,$$

where c is a positive constant. It is also possible to choose independent adaptation steps for each filter coefficient in the LMS algorithm, e.g., according to,

w(n+1)=w(n)+Aū(n)e(n),

where A is given by,

$$A = [ \begin{matrix}{α}_{1} & 0 & 0 & … & 0 \\ 0 & {α}_{2} & 0 & … & 0 \\ 0 & 0 & {α}_{3} & … & 0 \\ ⋮ & ⋮ & ⋮ & ⋱ & ⋮ \\ 0 & 0 & 0 & … & {α}_{M}\end{matrix} ] .$$

If instead the following cost function

J(n)=E{|e(n)|}

is used, then the update equation becomes

w(n+1)=w(n)+αsign[e(n)] u̅(n).

This adaptive filter is called the Sign LMS, which is used in applications with extremely high requirements on low computational complexity.

Another adaptive filter is the Leaky LMS, which uses a constrained minimization with the following cost function

J(n)=E{|e(n)|²}+α∥w(n)∥².

This constraint has the same effect as if white noise with variance a was added to the predicted signal profile u(n). As a result, the uncertainty in the input signal u(n) is increased, which tends to hold the filter coefficients back. The Leaky LMS is preferably used when R, the correlation matrix of u(n), has one or more eigenvalues equal to zero. However, in systems without noise, the Leaky LMS makes performance poorer. The update equation of the filter coefficients for the Leaky LMS is given by,

w(n+1)=(1−μα)w(n)+μ u̅(n)e(n).

Instead of minimizing the MSE cost function as above, the Recursive Least Squares (RLS) adaptive filter algorithm minimizes the following cost function

$$J ( n ) = {\sum }_{i = 1}^{n} {λ}^{n - i} {\vert e ( i ) \vert}^{2} ,$$

where λ is called forgetting factor, 0<λ≦1, and the method is called Exponentially Weighted Least Squares. It may be shown that the update equations of the filter coefficients for the RLS algorithm are, after the following initialization

w(0)=0_{M×1}

P(0)=δ⁻I_{M×M}

where I_{M×M} is the identity matrix M×M, given according to

$$k ( n ) = \frac{{λ}^{- 1} P ( n - 1 ) \overline{u} ( n )}{1 + {λ}^{- 1} {\overline{u}}^{T} ( n ) P ( n - 1 ) \overline{u} ( n )}$$

$$ξ ( n ) = d ( n ) - {w}^{T} ( n - 1 ) \overline{u} ( n )$$

$$w ( n ) = w ( n - 1 ) + k ( n ) ξ ( n )$$

$$P ( n ) = {λ}^{- 1} P ( n - 1 ) - {λ}^{- 1} k ( n ) {\overline{u}}^{T} ( n ) P ( n - 1 ) ,$$

where δ is a small positive constant for high signal-to-noise ratio (SNR), and a large positive constant for low SNR, δ<<0.01σ_{μ}², and ξ(n) corresponds to e(n) in the preceding algorithms. During the initialization phase the following cost function

$$J ( n ) = {\sum }_{i = 1}^{n} {λ}^{n - i} {\vert e ( i ) \vert}^{2} + {δλ}^{n} {‖ w ( n ) ‖}^{2} ,$$

is minimized instead, due to the use of the initialization P(0)=δ⁻¹I. The RLS algorithm converges in approximately 2M iterations, which is considerably faster than for the LMS algorithm. Another advantage is that the convergence of the RLS algorithm is independent of the eigenvalues of R, which is not the case for the LMS algorithm.

Several RLS algorithms running in parallel may be used with different λ and δ, which may be combined in order to improve performance, i.e., λ=1 may also be used in the algorithm (steady state solution) with many different δ:s.

It should be noted that both the LMS algorithm and the RLS algorithm can be implemented in fixed-point arithmetic, such that they can be run on a processor that has no floating point unit, such as a low-cost embedded microprocessor or microcontroller.

To illustrate the effectiveness of the removal process using an adaptive filter, the top graph in FIG. A12(a) illustrates the error signal e(n) output by the adaptive filter structure in FIG. A11, using an RLS algorithm as adaptive update algorithm 32, operating on a measurement signal from the venous sensor 4a in FIG. A4, at a flow rate of 430 ml/min. The adaptive filter structure is provided with a predicted signal profile obtained in a reference measurement at the same flow rate. The RLS algorithm, designed with M=15, converges after about 2M, which equals 3 seconds with the current sampling frequency of 10 Hz. The top graph thus shows the measurement signal after elimination of the first pulses. The bottom graph in FIG. A12(a) is included for reference, and shows the measurement signal from the venous sensor 4a while the blood pump 3 is stopped. Clearly, the adaptive filtering is operable to provide, after a convergence period, a monitoring signal that properly represents the second pulses.

FIG. A12(b) corresponds to FIG. A12(a), but is obtained for a measurement signal from the arterial sensor 4b in FIG. A4.

Irrespective of implementation, the performance of the adaptive filter 30 (FIG. A11) may be further improved by switching the adaptive filter 30 to a static mode, in which the update algorithm 34 is disabled and thus the filter coefficients of the filter 32 (FIG. A11) are locked to a current set of values. The switching of the adaptive filter 30 may be controlled by an external process that analyses the second pulses in the error signal e(n), typically in relation to first pulse data. The first pulse data may be obtained from the measurement signal, a reference signal (see above), a dedicated pulse sensor, a control unit for the first pulse generator, etc. The adaptive filter 30 may be switched into the static mode if the external process reveals that the rate of second pulses starts to approach the rate of the first pulses and/or that the amplitude of the second pulses is very weak (in relation to an absolute limit, or in relation to a limit given by the amplitude of the first pulses). The adaptive filter may remain in static mode for a predetermined time period, or until released by the process.

In a variant, a predicted signal profile of the second pulses (denoted “predicted second profile”) is used as input signal to the adaptive filter 30 (instead of the predicted signal profile of the first pulses), and the monitoring signal is formed by the estimated measurement signal \hat{d}(n) (instead of the error signal e(n)). The foregoing discussion with respect to adaptive filters is equally applicable to this variant.

The invention has mainly been described above with reference to a few embodiments. However, as readily appreciated by a person skilled in the art, other embodiments than the ones disclosed above are equally possible with the scope and spirit of the invention, which is defined and limited only by the appended patent claims.

For example, the measurement and reference signals may originate from any conceivable type of pressure sensor, e.g. operating by resistive, capacitive, inductive, magnetic or optical sensing, and using one or more diaphragms, bellows, Bourdon tubes, piezo-electrical components, semiconductor components, strain gauges, resonant wires, accelerometers, etc.

Although FIG. A1 indicates that the pressure sensor 4a-4c is connected to the first sub-system S1, it may instead be connected to measure the fluid pressure in the second sub-system S2. Further, the fluid containing system need not be partitioned into first and second sub-systems S1, S2 connected via a fluid connection C, but could instead be a unitary fluid containing system associated with a first pulse generator and a second pulse generator, wherein the each pressure sensor is arranged in the fluid containing system to detect a first pulse originating from the first pulse generator and a second pulse originating from the second pulse generator.

Further, the inventive technique is applicable for monitoring in all types of extracorporeal blood flow circuits in which blood is taken from the systemic blood circuit of the patient to have a process applied to it before it is returned to the patient. Such blood flow circuits include circuits for hemodialysis, hemofiltration, hemodiafiltration, plasmapheresis, apheresis, extracorporeal membrane oxygenation, assisted blood circulation, and extracorporeal liver support/dialysis. The inventive technique is likewise applicable for monitoring in other types of extracorporeal blood flow circuits, such as circuits for blood transfusion, infusion, as well as heart-lung-machines.

The inventive technique is also applicable to fluid systems containing other liquids than blood.

Further, the inventive technique is applicable to remove pressure pulses originating from any type of pumping device, not only rotary peristaltic pumps as disclosed above, but also other types of positive displacement pumps, such as linear peristaltic pumps, diaphragm pumps, as well as centrifugal pumps. In fact, the inventive technique is applicable for removing pressure pulses that originate from any type of pulse generator, be it mechanic or human.

Likewise, the inventive technique is applicable to isolate pressure pulses originating from any type of pulse generator, be it human or mechanic.

The inventive technique need not operate on real-time data, but could be used for processing off-line data, such as a previously recorded measurement signal.

### End Appendix A

### APPENDIX B

### Brief Description of the Drawings

Embodiments of the inventive concepts will now be described in more detail with reference to the accompanying schematic drawings.

FIG. B1 is a schematic view of a general fluid arrangement in which the inventive concepts may be used for monitoring the integrity of a fluid connection.

FIG. B2 is a flow chart of a monitoring process according to a first inventive concept.

FIG. B3(a) is a plot of the measurement signal as a function of time, FIG. B3(b) is a plot of the measurement signal in FIG. B3(a) after filtering, and FIG. B3(c) illustrates a statistical dispersion measure calculated for a sequence of time windows in the signal in FIG. B3(b).

FIG. B4(a) illustrates a matching procedure between a measurement signal and a predicted signal profile, FIG. B4(b) illustrates the position of best match, and FIG. B4(c) is a correlation curve resulting from the matching procedure in FIG. B4(a).

FIG. B5(a) is a plot of a signal segment containing a second pulse, and FIG. B5(b) is plot of an evaluation segment generated by averaging ten signal segments.

FIG. B6 is a flow chart of a monitoring process according to a second inventive concept.

FIG. B7(a)-7(d) illustrate processing of candidate pulses identified in a measurement signal.

FIG. B8 is a flow chart of part of a monitoring process according to the second inventive concept.

FIG. B9 is a flow chart of a monitoring process that combines the first and second inventive concepts.

### Detailed Description of Inventive Concepts and Embodiments

In the following, inventive concepts and associated embodiments will be described with reference to fluid containing systems in general. Thereafter, the inventive concepts will be further exemplified in the context of systems for extracorporeal blood treatment.

Throughout the following description, like elements are designated by the same reference signs.

### General

FIG. B1 illustrates a general fluid arrangement in which a fluid connection C is established between a first fluid containing system S1 and a second fluid containing system S2. The fluid connection C may or may not transfer fluid from one system to the other. A first pulse generator 3 is arranged to generate a series of pressure waves in the fluid within the first system S1, and a second pulse generator 3′ is arranged to generate a series of pressure waves in the fluid within the second system S2. A pressure sensor 4c is arranged to measure the fluid pressure in the first system S1. As long as the fluid connection C is intact, pressure waves generated by the second pulse generator 3′ will travel from the second system S2 to the first system S1, and thus second pulses originating from the second pulse generator 3′ will be detected by the pressure sensor 4c in addition to first pulses originating from the first pulse generator 3. It is to be noted that either one of the first and second pulse generators 3, 3′ may include more than one pulse-generating device. Further, any such pulse-generating device may or may not be part of the respective fluid containing system S1, S2.

The fluid arrangement of FIG. B1 further includes a surveillance device 25 which is connected to the pressure sensor 4c, and possibly to one or more further pressure sensors 4a, 4b, as indicated in FIG. B1. Thereby, the surveillance device 25 acquires one or more measurement signals that are time-dependent to provide a real time representation of the fluid pressure in the first system S1. The surveillance device 25 monitors the integrity of the fluid connection C, based on the principle that the presence of second pulses indicates that the fluid connection C is intact, whereas absence of second pulses indicates that the fluid connection C is compromised. The absence of second pulses may bring the surveillance device 25 to issue an alarm or warning signal, and/or alert a control system of the first or second fluid containing systems S1, S2 to take appropriate action.

The surveillance device 25 is thus configured to continuously process the time-dependent measurement signal(s) to determine whether second pulses are present or not. Typically, the determination involves analyzing the measurement signal(s), or a pre-processed version thereof, in the time domain to calculate a value of an evaluation parameter which is indicative of the presence or absence of second pulses in the measurement signal(s). Depending on implementation, the surveillance device 25 may use digital components or analog components, or a combination thereof, for receiving and processing the measurement signal(s).

In the context of the present disclosure, “absence” of a pulse may imply that the pulse has disappeared, or at least that it has decreased sufficiently in magnitude compared to the pulse deemed to be “present”. The assessment of presence or absence may involve calculating an evaluation parameter value based on the measurement signal(s) and comparing the parameter value to a threshold value.

### First Inventive Concept

FIG. B2 is a flow chart that illustrates steps of a monitoring process according to a first inventive concept. A measurement signal is received (step 201) and subjected to a filtering process (step 202) that essentially removes the first pulses from the measurement signal, while leaving at least part of the second pulses intact. The filtered measurement signal is then subjected to a time domain analysis (step 203), in which a value of an evaluation parameter is calculated based on signal values within a time window in the filtered measurement signal, which is denoted “evaluation segment” in the following. The calculation is typically designed such that the evaluation parameter represents the distribution of signal values within the evaluation segment. Based on the resulting value of the evaluation parameter, it is decided (step 204) whether the fluid connection is intact or not, typically by comparing the resulting value to a threshold value.

For continuous surveillance, a time sequence of evaluation parameter values is calculated based on a time sequence of evaluation segments obtained from the measurement signal. These evaluation segments may be overlapping or non-overlapping in time. In one embodiment, individual sections of the measurement signal are acquired, filtered and analyzed, one after the other. Each evaluation segment may correspond to one such section of the measurement signal; the time window is thus applied already when the measurement signal is acquired. In another embodiment, the measurement signal is continuously acquired and filtered, whereupon evaluation segments are extracted from the filtered signal and analyzed.

FIG. B3(a) shows an example of a time-dependent measurement signal containing first and second pulses with a relative magnitude of 10:1. The first and second pulses have a frequency of 1 Hz and 1.33 Hz, respectively. FIG. B3(b) shows the time-dependent measurement signal after removal of the first pulses, leaving only second pulses and noise. It should be noted that there is an absence of second pulses after about 4 seconds. FIG. B3(c) illustrates a variance measure calculated for a sequence of non-overlapping time windows in the filtered measurement signal in FIG. B3(b), each time window being about 0.75 seconds. Clearly, by using the variance measure as an evaluation parameter, it is possible to detect the absence of the second pulse at the time point of about 4 seconds. An exemplifying threshold value is indicated by a dotted line.

The first inventive concept has the potential of providing a comparatively robust measure of the integrity of the fluid connection C. By analyzing the temporal distribution of signal values within the evaluation segment, an improved tolerance to noise and disturbing signals may be obtained.

Furthermore, compared to techniques that rely on frequency domain analysis of the measurement signal for detecting the presence of second pulses, the first inventive concept may provide an improved tolerance to variations in the pulse repetition interval of the second pulse generator 3′, since the first inventive concept relies on a time domain analysis. Such variations may occur, e.g., when the second pulse generator 3′ is a human heart, and the second system S2 thus is the blood system of a human Variations in heart rhythm (heart rate variability, HRV) will cause the peak from the heart in the frequency domain to be smeared out, making it harder to detect. In healthy subjects under calm conditions, HRV may be as large as 15%. Unhealthy subjects may suffer from severe heart conditions such as atrial fibrillation and supraventricular ectopic beating, which may lead to an HRV in excess of 20%, and ventricular ectopic beating, for which HRV may be in excess of 60%. These heart conditions are not uncommon among, e.g., dialysis patients.

As long as the time window is selected such that each evaluation segment contains at least one second pulse, the presence/absence of second pulses will affect the evaluation parameter, if properly chosen. A fixed-length time window may be used, with the length of the time window being chosen with respect to a maximum pulse repetition rate of the second pulse generator 3′. The length of the time window may be set by constraints in the second pulse generator 3′ or by a selected performance limit of the surveillance method. Alternatively, the length of the time window and/or the location of the time window in the filtered measurement signal may be selected based on a predicted timing of the second pulse(s) to be detected. The acquisition and use of such a predicted timing (“timing information”) will be further exemplified below with reference to the second inventive concept.

Still further, the time domain analysis according to the first inventive concept may allow for faster detection than a frequency domain analysis, since the former may have the ability to detect a single second pulse in the evaluation segment whereas the generation of a frequency spectrum requires a greater number of second pulses in the evaluation segment. Thus, frequency domain analysis may be associated with a greater time lag than time domain analysis.

The evaluation parameter may be calculated as a statistical dispersion measure of the signal values within the evaluation segment. Non-limiting examples of potentially useful statistical dispersion measures include standard deviation (σ), variance (σ²), coefficient of variation (σ/μ) and variance-to-mean (σ²/μ). Other examples include a sum of differences, e.g. given by

$${\sum }_{i = 2}^{n} \vert {x}_{i} - {x}_{i - 1} \vert , or {\sum }_{i = 1}^{n} {\sum }_{j = 1}^{n} \vert {x}_{i} - {x}_{j} \vert ,$$

or an energy measure, such as

$${\sum }_{i = 1}^{n} {x}_{i}^{2} ,$$

with n being the number of signal values x in the evaluation segment. Yet other examples include a measure based on a sum of absolute differences from an average value m, with the average value m being calculated for the signal values in the evaluation segment using any suitable function, such as arithmetic mean, geometric mean, median, etc. It is to be noted that all of the above suggested dispersion measures also include normalized and/or weighted variants thereof.

As an alternative or supplement to calculating a statistical dispersion measure, the evaluation parameter may result from a matching procedure, in which the evaluation segment is matched to one or more predicted signal profiles of a second pulse. Preferably, but not necessarily, each predicted signal profile represents a single second pulse. Typically, the matching procedure involves convolving or cross-correlating the evaluation segment and the predicted signal profile, and the evaluation parameter value is a resulting correlation value, typically the maximum correlation value.

A matching procedure based on cross-correlation is further exemplified in FIGS. B4(a)-B4(c). The matching procedure is used to distinguish between the hypotheses

H₀: x(n)=w(n)

H₁: x(n)=s(n)+w(n)

with x(n) being the evaluation segment, w(n) being an error signal representing disturbances introduced by noise/signal interference/measurement errors, etc, and s(n) being the predicted signal profile of the second pulse. If H₁ is deemed more likely than H₀, then a second pulse has been identified and the fluid connection C is deemed intact. If H₀ is deemed more likely than H₁, then a second pulse cannot be identified and the fluid connection C may be compromised.

FIG. B4(a) is a graph showing an example of a predicted signal profile s(n) and an evaluation segment x(n). In this particular example, the evaluation segment has a signal-to-noise ratio (SNR) of 4.8 dB, i.e. the energy of the signal profile s(n) is 3 times the energy of the error signal w(n). During the cross-correlation, the signal profile s(n) is slid in a number of time steps along the time axis, as indicated by arrow in FIG. B4(a), and the integral of the product s(n)·x(n) is calculated for each time step. The cross-correlation thus results in a time sequence of correlation values, with the maximum correlation value indicating the time point of best match between x(n) and s(n). FIG. B4(b) illustrates the relative position between x(n) and s(n) at the time point for best match, and FIG. B4(c) illustrates the resulting correlation values as a function of said time steps. The magnitude of the maximum correlation value, optionally calculated as a weighted average within a range around the maximum correlation value (cₘₐₓ), may thus be used to distinguish between the above hypotheses.

As indicated in FIG. B4(c), the matching procedure not only identifies the presence of a second pulse, it also provides an indication of the location of the second pulse in the evaluation segment, given by the time point (tₚ) for the maximum correlation value (cₘₐₓ). This time point may be used to assess the reliability of the determined maximum correlation value, by comparing this time point to a predicted time point. Such a predicted time point may be obtained from aforesaid timing information, as will be further explained below in relation to the second inventive concept.

The predicted signal profile may be generated as an average of a number of recordings of second pulses. For example, it may be generated by averaging a number of evaluation segments, before and/or during the monitoring process.

To improve the signal quality of the predicted profile, with or without averaging, the measurement signal may be acquired while the first pulse generator is stopped, whereby the measurement signal is free of first pulses. Thus, the first pulse generator may be intermittently stopped during the monitoring process for calculation of an updated signal profile of the second pulses.

In another variant, the predicted signal profile is obtained from one or more reference signals originating from a reference pressure sensor (e.g. any one of pressure sensors 4a-4c in FIG. B1) in the first system. Such a reference pressure sensor is suitably arranged to detect second pulses even if the fluid connection is compromised, e.g. via a second fluid connection between the first and second fluid containing systems. The reference pressure sensor may be installed to be isolated from the first pulses, such that the reference signal is essentially free of first pulses. Alternatively, if the reference signal includes both first and second pulses, the reference signal may be subjected to a filtering process (e.g. according to step 202 in FIG. B2) to remove the first pulses while leaving the second pulses intact in the reference signal. An example of such a reference pressure sensor is an arterial pressure sensor in an extracorporeal blood flow circuit. In such an extracorporeal blood flow circuit, the measurement signal(s) may originate from one or more venous pressure sensors, e.g. if the monitoring process aims at monitoring the integrity of the venous-side fluid connection between the extracorporeal blood flow circuit and a patient.

In one specific implementation, the reference signal is obtained continuously or intermittently during the monitoring process, and the predicted signal profile is continuously or intermittently calculated based on the reference signal. Thus, in the context of the above-mentioned extracorporeal blood flow circuit, the integrity of the venous-side fluid connection may be monitored by continuously matching evaluation segments from the venous pressure sensor against a predicted signal profile obtained from the arterial pressure sensor. It is even conceivable that the predicted signal profile is updated for each evaluation segment (denoted “synchronous monitoring” in the following). The matching procedure may benefit from the use of timing information, as will be further explained below in relation to the second inventive concept. Alternatively, the predicted signal profile may be pre-generated, e.g. by averaging recordings of second pulses from a number of fluid arrangements, similar to the one that is being monitored (cf. FIG. B1). Optionally, such a pre-generated signal profile may be adapted to specifics of the fluid arrangement to be monitored, by applying a mathematical model taking into account arrangement-specific parameters, such a type of fluid connection, flow rate, fluid characteristics, etc. Alternatively, the predicted signal profile may be obtained entirely by mathematical modelling based on arrangement-specific parameters. According to yet another alternative, a standard profile is used as predicted signal profile, e.g. a bell-shaped function such as a Gaussian distribution function.

In order to improve the detection of second pulses, it is conceivable to subject the filtered measurement signal/evaluation segment to a signal enhancement process, which removes high-frequency components (cf. error signal w(n)), before calculation of the evaluation parameter value. Such a signal enhancement process may involve subjecting the filtered measurement signal/evaluation segment to a low-pass filtering. However, a more significant improvement in SNR of the evaluation segment may be achieved by averaging several consecutive second pulses in the filtered measurement signal, again based on the above-mentioned predicted timing of the second pulse(s) (i.e. timing information). Such a signal enhancement process would thus involve using the predicted timing to identify a set of second pulse segments in the filtered measurement signal, aligning the second pulse segments in the time domain based on the predicted timing, and generating an average representation by summing the aligned signal values for each time value in the time domain. Optionally, the average representation is normalized by the number of second pulse segments to generate a true average. The average representation may then be used as the above-mentioned evaluation segment, or the evaluation segment may be extracted from a time window within the average representation.

The signal enhancement process is further exemplified in FIGS. B5(a)-B5(b). FIG. B5(a) is a time domain representation of a filtered measurement signal x(n)=s(n)+w(n) with a SNR of −9 dB, i.e. the energy of the error signal w(n) is 8 times the energy of the signal profile s(n), making time domain analysis for detection of the second pulse difficult, if not impossible. FIG. B5(b) is a time domain representation after averaging of 10 different second pulse segments similar to the one in FIG. B5(a). Clearly, the SNR has been improved significantly, allowing a second pulse to be detected using time domain analysis.

It is to be understood that the monitoring process of FIG. B2 may operate on more than one measurement signal, if the fluid arrangement to be monitored includes more than one pressure sensor (cf. 4a, 4b in FIG. B1). In such a configuration, the above-described signal enhancement process may involve using aforesaid timing information to identify and average second pulse segments from at least two filtered measurement signals originating from different pressure sensors. Thus, the second pulse segments may be extracted from plural time windows in each measurement signal, and/or from one or more time windows in different measurement signals.

The filtering process according to step 202 in FIG. B2 aims at removing the first pulses from the measurement signal to such an extent that the second pulses can be detected by the subsequent time domain analysis (step 203). For example, a comb filter and/or a combination of band-stop or notch filters, typically cascade coupled, may be operated on the measurement signal to block out all frequency components originating from the first pulse generator 3. Alternatively, such blocking may be achieved by the use of one or more adaptive filters and notch-equivalent filters, e.g. as disclosed in aforesaid WO 97/10013. In yet another alternative embodiment, the measurement signal is processed in the time domain to cancel the first pulses. In such an embodiment, a standard signal profile of the first pulses may be obtained, which is then subtracted from the measurement signal at suitable amplitude and phase. The phase is indicated by phase information which may be obtained from a signal generated by a phase sensor coupled to the first pulse generator 3, or from a control signal for the first pulse generator 3. The standard signal profile may be obtained from one or more of the pressure sensors 4a-4c in the first fluid containing circuit S1, suitably by identifying and averaging a set of first pulse segments in the measurement signal(s) similarly to the above-mentioned signal enhancement process. The standard signal profile may or may not be updated intermittently during the monitoring process. Alternatively, a predetermined standard signal profile is used, which optionally may be modified according to a mathematical model accounting for wear in the first pulse generator, fluid flow rates, tubing dimensions, speed of sound in the fluid, etc. It should be noted that by filtering the measurement signal in the time domain, instead of the frequency domain, it is possible to eliminate the first pulses and still retain the second pulses, even if the first and second pulses overlap in the frequency domain

### Second Inventive Concept

FIG. B6 is a flow chart that illustrates steps of a monitoring process according to a second inventive concept. In this process, a measurement signal is received (step 601) and timing information is obtained, from the measurement signal or otherwise (step 602). The timing information is indicative of the timing of second pulses in the measurement signal. Subsequently, the measurement signal is processed (step 603) based on the timing information, to calculate a value of an evaluation parameter which is indicative of the presence or absence of a second pulse in the measurement signal. Based on the resulting value of the evaluation parameter, it is decided (step 604) whether the fluid connection is intact or not, typically by comparing the resulting value to a threshold value.

Thus, in the second inventive concept, timing information indicates the expected position of a second pulse in the measurement signal. This additional information may allow the second pulse to be identified from other types of signal features, e.g. different/simpler evaluation parameters, and/or it may allow for an increased reliability in detecting presence/absence of second pulses.

Furthermore, as explained above, the provision of timing information allows for signal enhancement by identifying and averaging second pulse segments in one or more measurement signals. The signal enhancement may increase the SNR of the measurement signal, allowing for the use of a rudimentary measure as evaluation parameter, such as signal amplitude, local maximum, local average, etc. This may serve to improve the processing speed and/or allow for less sophisticated detection equipment.

It is to be understood that the second inventive concept can be combined with any of the features of the first inventive concept. For example, the measurement signal may be filtered to remove first pulses, and the evaluation parameter may be calculated for an evaluation segment given by signal values within a time window in the filtered measurement signal. Also, any one of the evaluation parameters suggested in relation to the first inventive concept is equally applicable to the second inventive concept. It is to be noted, however, that the filtering of the measurement signal is not an essential feature of the second inventive concept, since the use of timing information may allow second pulses to be detected in the measurement signal even in the presence of first pulses.

The second inventive concept may also improve the detection speed, since the timing information may provide a predicted time point for the second pulse in the measurement signal/filtered measurement signal/evaluation segment. Thereby, the number of signal values that need to be processed for calculation of the evaluation parameter value may be reduced. For example, the aforesaid matching procedure may be simplified, since the correlation between the predicted signal profile and the evaluation segment need only be calculated for the predicted time point, or a confined time range around this predicted time point. Correspondingly, the calculation of a statistical dispersion measure or the above-mentioned rudimentary measure may be simplified, since the provision of timing information makes it possible to reduce the size of the time window for extracting the evaluation segment, while still ensuring that each evaluation segment includes at least one second pulse. For example, the size of the time window may be reduced if the timing information indicates a shortened pulse interval between the second pulses, and/or the time window may be centred on the predicted time point of each second pulse.

Still further, the second inventive concept allows for assessing the reliability of a calculated evaluation parameter value, by comparing a time point associated with the evaluation parameter value with a predicted time point given by the timing information. For example, the time point for a maximum correlation value obtained in the aforesaid matching procedure may be compared with a predicted time point for a second pulse. If these time points deviate too much, the monitoring process may determine that a second pulse is absent, even though the magnitude of the correlation value might indicate presence of a second pulse.

The timing information may be obtained in any one of a plurality of different ways. For example, the timing information may be extracted from the output signal of a pulse sensor coupled to the second fluid containing system. The output signal may indicate individual second pulses or an average time between second pulses. In either case, a predicted time point for a second pulse in the measurement signal can be calculated based on the output signal of the pulse sensor and a known difference in arrival time between the pulse sensor and the pressure sensor(s) that generates the measurement signal(s). The pulse sensor may sense the pressure waves that are generated in the fluid by second pulse generator, or it may directly reflect the pulse generation process in the second pulse generator, e.g. via a control signal for the second pulse generator or a pulse rate meter mechanically coupled to the second pulse generator. In one application, to be further exemplified below, the second fluid containing system is a blood system of a human, and the pulse generator is a human heart. In such an application, the timing information may be provided by any conventional pulse sensor such as a pulse watch, a pulse oximeter, an electrocardiograph, etc.

Alternatively, the timing information may be obtained based on the relative timing of previously detected second pulses in the measurement signal, e.g. given by the time points associated with previously calculated evaluation parameter values. For example, the time difference between the two most recently detected second pulses may be used to predict the time point for subsequent second pulse(s).

Alternatively, the timing information may be obtained from one or more reference signals originating from a reference pressure sensor in the first system. Such a reference pressure sensor is suitably arranged to detect second pulses even if the fluid connection is compromised, e.g. via a second fluid connection between the first and second fluid containing systems.

An example of such a reference pressure sensor is an arterial pressure sensor in an extracorporeal blood flow circuit. In such an extracorporeal blood flow circuit, the measurement signal(s) may originate from one or more venous pressure sensors, e.g. if the monitoring process aims at monitoring the integrity of the venous-side fluid connection between the extracorporeal blood flow circuit and a patient. The reference signal may be processed for detection of at least one second pulse, using any suitable technique, including the time domain techniques disclosed herein. The time point of the detected second pulse in the reference signal can then be converted to a predicted time point in the measurement signal/filtered measurement signal/evaluation segment using a known/measured difference in pulse arrival/transit time between the reference sensor and the pressure sensor(s) used for monitoring. Thus, in one embodiment, the difference in transit time is given by a fixed and predefined value.

In another embodiment, the difference in transit time between a blood line on the arterial side and a blood line on the venous side in the extracorporeal blood flow circuit is determined based on the actual arterial and venous pressures (absolute, relative, or average), which may be derived from any suitable sensor in the extracorporeal blood flow circuit (including the venous and arterial pressure sensors). The transit time decreases if the pressure increases, i.e., high pressure equals short transit time. During operation of the extracorporeal blood flow circuit, the venous pressure should be higher than the arterial pressure, and thus the transit time should be shorter in the venous blood line compared to the transit time in the arterial blood line. The difference in transit time may be determined based on, e.g., a physical model or a look-up table. The model/table may not only include information about pressure (absolute, relative, or average), but also information about material (elasticity, plasticity, etc), geometry (length, diameter, wall thickness, etc), temperature (both fluids and ambient temperature), mechanical factors (clamp, tension, actuators, kinking/occlusion, etc), fluid properties (viscosity, water/blood, chemical composition, etc), etc. The thus-determined difference in transit time may then be used to relate a time point of a detected second pulse in the reference signal from the arterial pressure sensor to a predicted time point in the measurement signal/filtered measurement signal/evaluation segment originating from the venous pressure sensor.

In a variant, an improved estimation of the timing information may be obtained by aligning and adding the filtered measurement signal/evaluation segment (derived from the venous pressure signal) with a correspondingly filtered reference signal (derived from the arterial pressure signal), to thereby calculate an average time-dependent signal with improved SNR. The aligning may be based on the aforesaid difference in transit time, given by the actual arterial and venous pressures (absolute, relative, or average). By identifying one or more second pulse(s) in the average time-dependent signal, an improved estimation of the timing information is obtained.

Alternatively or additionally, to potentially improve the precision of the timing information, the timing information may be obtained by intermittently stopping the first pulse generator, while identifying at least one second pulse in the reference signal or the measurement signal.

Optionally, the process of obtaining timing information based on an identified second pulse, be it in the reference signal or the measurement signal, may involve validating the identified second pulse (a candidate pulse) against a temporal criterion. Such a temporal criterion may, e.g., indicate an upper limit and/or a lower limit for the time difference between the time point for the candidate pulse and one or more previously identified (and suitably validated) second pulses. These limits may be fixed, or they may be set dynamically in relation to a preceding time difference. Any candidate pulse that violates the temporal criterion may be removed/discarded from use in obtaining the timing information.

In yet another alternative, the timing information is obtained from a measurement signal using an iterative approach. In this iterative approach, the measurement signal is processed to calculate a time-sequence of evaluation parameter values, e.g. based on the first inventive concept. These evaluation parameter values identify a sequence of candidate pulses and associated candidate time points, which is validated against a temporal criterion. Such a temporal criterion may, e.g., indicate an upper limit and/or a lower limit for the time difference between the candidate time points. The temporal criterion may be given by constraints in the second pulse generator 3′. Any candidate time points that violate the temporal criterion may be removed/discarded, and the timing information may be obtained from the remaining time points.

Different validation methods may be used depending on the availability of previous timing information, i.e. information about time points of preceding second pulses. Such previous timing information may be given by any one of the methods described in the foregoing, or resulting from a previous iteration of the iterative approach.

FIG. B7(a) illustrates a sequence of candidate pulses (denoted by X), as well as a sequence of preceding second pulses (denoted by Y), laid out on a time axis. In a first validation step, predicted time points (arrows 1 in FIG. B7(b)) are calculated based on the previous timing information (e.g. second pulses Y). In a second validation step, a first temporal criterion is applied to remove/discard any candidate pulses that lie too far from the predicted time points, as also shown in FIG. B7(b). In a third validation step, a second temporal criterion is applied to retain only the candidate pulse with the largest evaluation parameter value among any candidate pulses that lie too close to each other, as shown in FIG. B7(c).

A different validation method may be used if previous timing information is not available. FIG. B8 is a flow chart for such a validation method. The initial step 801 of identifying candidate pulses is followed by a first validation step 802, in which a first temporal criterion is applied to retain only the candidate pulse with the largest evaluation parameter value among any candidate pulses that lie too close to each other. FIG. B7(d) shows an exemplifying result of applying the first validation step 802 to the sequence of candidate pulses in FIG. B7(a). Then, in step 803, different combinations of the remaining candidate pulses are formed. In step 804, an average representation is calculated for each such combination, by aligning and summing corresponding signal segments of the measurement signal/filtered measurement signal. The combinations may be formed based on a second temporal criterion that defines an upper limit and/or a lower limit for the time difference between the candidate pulses. In a second validation step 805, an evaluation parameter value is calculated for each such average representation, and the maximum evaluation parameter value is extracted. Finally, in step 806, it is decided whether the fluid connection is intact or not, by comparing the maximum evaluation parameter value to a threshold value. If the maximum evaluation parameter value exceeds the threshold value, it may be concluded that a second pulse is present and that the fluid connection is intact. It may be noted that there is no need to explicitly extract the timing information in the validation method in FIG. B8, since the use of the timing information is embedded in the final step 806 of determining the integrity of the fluid connection.

It should also be noted that different evaluation parameters and/or threshold values may be used in steps 801 and 806. It is also conceivable to use a combination of two or more of the above alternative methods for obtaining the timing information.

FIG. B9 is a flow chart of an embodiment that combines features of the first and second inventive concepts. Specifically, a measurement signal is obtained and filtered according to steps 201 and 202 of the first inventive concept. Then, in step 202′, the filtered measurement signal is processed for signal enhancement, based on timing information. As discussed above in relation to FIG. B5, step 202′ typically involves identifying, aligning and summing a set of second pulse segments in the filtered measurement signal, to create an average signal representation. An evaluation parameter value is then calculated based on the enhanced signal representation according to step 203/603 of the first/second inventive concept, and it is decided whether the fluid connection is intact or not (steps 204/604). The method also involves receiving a measurement signal (which may be the same measurement signal as in step 201, or the aforesaid reference signal) according to step 601 of the second inventive concept. Then, the measurement/reference signal is filtered to remove the first pulse, if required, according to step 202 of the first inventive concept. Finally, the timing information is obtained according to step 602 of the second inventive concept.

### Combinations of Monitoring Techniques

As explained in the foregoing, the technique for monitoring the integrity of the fluid connection can be based on either of the first and second inventive concepts, or a combination thereof. It is also possible to combine such an inventive monitoring technique with one or more conventional monitoring techniques, which e.g. involve the use of an air detector, or a comparison of average pressure levels with threshold values as described by way of introduction. Other conventional monitoring techniques are disclosed in aforesaid WO 97/10013 and US2005/0010118.

It might also be desirable to combine the inventive monitoring techniques with other techniques that are specially designed to handle adverse operating conditions. One such operating condition may arise when the first and second pulses overlap in the frequency domain. As discussed above in relation to step 202 of FIG. B2, such an operating condition could be handled by filtering the measurement signal in the time domain. However, the monitoring precision may be increased further by combining the inventive monitoring technique with a phase-locking technique or a beating detection method, to be described in the following.

The phase-locking technique involves controlling the first/second pulse generator 3, 3′ so as to synchronize the pulse rate of the first and second pulse generators 3, 3′ while applying a phase difference between the first and second pulses. Thereby, the first and second pulses will be separated in time, and can be detected using the time domain analysis according to the first and/or second inventive concepts. The phase difference may be approximately 180°, since this may maximize the separation of the first and second pulses in the time domain. The phase-locking technique may be activated when it is detected that the frequency of the second pulse generator approaches a frequency of the first pulse generator, or vice versa.

The beating detection method is an alternative or complementary monitoring technique which involves evaluating the presence or absence of a beating signal in the measurement signal to determine the integrity of the fluid connection. The beating signal manifests itself as an amplitude modulation of the measurement signal and is formed by interference between pressure waves generated by the first pulse generator and pressure waves generated by the second pulse generator. Instead of trying to identify second pulses in the measurement signal, the presence of second pulses is identified via the secondary effect of beating. Generally, beating is a phenomenon which is especially noticeable when two signals with closely spaced frequencies are added together. Thus, the beating signal detection is inherently well-suited to be used when the first and second pulses are closely spaced in the frequency domain. The beating signal may or may not be detected by analysing the measurement signal in the time domain. Suitably, the beating detection involves obtaining one or more specific frequencies related to the first pulse generator, and creating at least one filtered measurement signal in which all but one of said specific frequencies are removed. The beating signal may then be detected by determining an envelope of the filtered measurement signal. The beating detection method is the subject of Applicant's PCT publication WO2009/127683, which is incorporated herein in its entirety by reference.

It is to be understood that in any one of the above combinations, the different monitoring techniques may be carried out in series, in any order, or in parallel.

### Performance Improvements

The performance of the different methods for monitoring the integrity of a fluid connection as described herein may be improved by applying any of the following variations.

### Hypothesis Test

The determination of the integrity of the fluid connection between the first and second fluid containing systems could be represented by a hypothesis test. In this hypothesis test, the above-mentioned evaluation parameter value β is compared to a threshold. The output of the hypothesis is a decision, which may be “intact fluid connection” (H₁) if β>γ₁, “compromised fluid connection” (H₀) if β<γ₀, or “uncertain decision” if γ₀≦β≦γ₁, wherein γ₀ and γ₁ are different thresholds.

### Magnitude Dependent Monitoring Technique

The monitoring technique may be dynamically adjusted based on the magnitude of the first and/or second pulses in the measurement signal and/or in the reference signal. The dynamic adjustment may affect the process for obtaining timing information and/or the process for obtaining the parameter value based on the measurement signal.

For example, if the magnitude (e.g. amplitude) of second pulses in the reference signal are found to be smaller than the magnitude (e.g. amplitude) of second pulses in the measurement signal, or smaller than a predetermined absolute limit, the timing information may be obtained based on the measurement signal, whereas the timing information otherwise is obtained based on the reference signal (or vice versa). Thus, with reference to FIG. B9, step 601 is adjusted based on the magnitude of second pulses.

In another example, if the magnitude (amplitude) of the second pulses in the reference signal again are found to be too small, the monitoring method may switch to another method for detecting presence or absence of second pulses in the measurement signal, e.g. a method that operates without timing information (e.g. by omitting steps 601, 602, 202 and 202′ in FIG. B9).

In the above examples, if the magnitude of first and second pulses are covariant entities, the dynamic adjustment may alternatively be based on the magnitude of first pulses, or the magnitude of a combination of first and second pulses.

### Monitoring Technique Based on Patient Data Records

When the second fluid containing system (S2 in FIG. B1) is a blood system of a patient, the monitoring method may be configured to access and use patient-specific information, i.e. existing data records for the patient, e.g. obtained in earlier treatments of the same patient. The patient-specific information may be stored in an internal memory of the surveillance device (25 in FIG. B1), on an external memory which is made accessible to the surveillance device, or on a patient card where the information is e.g. transmitted wirelessly to the surveillance device, e.g. by RFID (Radio Frequency IDentification). For example, the surveillance device may compare the filtered measurement signal, or a parameter derived therefrom, to the patient-specific information. If large differences are identified, a warning may be issued and/or the monitoring technique may be modified (or chosen according to a predetermined table). Furthermore, the patient-specific information may be used by the surveillance device to optimize the monitoring technique by e.g. determining personal threshold values for use in the foregoing algorithms/processes. The patient-specific information may also be used by the surveillance device to determine if an alternative monitoring technique or combinations of monitoring techniques should be used.

Use of Information from Regular Stops of First Pulse Generator

In one embodiment, the first pulse generator is regularly (intermittently or periodically) stopped, and the measurement signal and/or reference signal is analysed for determination of amplitude, frequency and phase of second pulses. This resulting information may then be used to achieve detection by the above-mentioned phase-locking technique.

Alternatively or additionally, if the magnitude (e.g. amplitude) of the second pulse(s) detected during such a stop is smaller than a certain limit (chosen with a margin for safe detection), an alert on “uncertain detection” may be issued. Alternatively, if the magnitude is smaller than another limit, the first pulse generator may be actively controlled to be stopped at specific time intervals, where the information obtained during each stop may be used to modify the monitoring technique. For example, the thus-obtained information may be used to change (or add) threshold values in the foregoing algorithms/processes, or to determine if an alternative monitoring technique or combinations of monitoring techniques should be used. In another example, if the thus-obtained information indicates the pulse rate of second pulses, a dedicated bandpass filter (e.g. centred on the thus-obtained pulse rate) may be operated on the measurement signal/filtered measurement signal/evaluation segment to further improve the input to the process for obtaining timing information (cf. step 602 in FIG. B6) and/or the process for obtaining the parameter value based on the measurement signal (cf. step 203/603 in FIGS. B2 and B9). In one embodiment, such a bandpass filter is applied if the rates of first and second pulses are found to differ by more than a certain limit, e.g. about 10%.

In another embodiment, the first pulse generator is selectively controlled so as to reduce the flow rate through the fluid arrangement. By reducing the flow rate, it is possible to accept a longer response time of the monitoring process to a fault condition, while such a longer response time may serve to improve the precision of the monitoring process in detecting fault conditions.

The invention has mainly been described above with reference to a few embodiments. However, as is readily appreciated by a person skilled in the art, other embodiments than the ones disclosed above are equally possible within the scope and spirit of the invention, which is defined and limited only by the appended patent claims.

The inventive monitoring techniques are applicable when the measurement signal originates from a pressure sensor arranged to sense the pressure in an extracorporeal blood flow circuit. In such an embodiment, the first fluid containing system (S1) is the extracorporeal blood flow circuit, the second fluid containing system (S2) is human blood system, and the fluid connection (C) may be formed by a connection between an access device and a blood vessel access. The first pulses may originate from the pumping device in the extracorporeal blood flow circuit (and/or any other pulse generator within or associated with the extracorporeal blood flow circuit), and the second pulses may originate from the human heart, and the integrity of the fluid connection is determined by applying the first and/or second inventive concepts to detect the presence/absence of the second pulses in the measurement signal.

The pressure (measurement) signal may originate from any conceivable type of pressure sensor, e.g. operating by resistive, capacitive, inductive, magnetic or optical sensing, and using one or more diaphragms, bellows, Bourdon tubes, piezo-electrical components, semiconductor components, strain gauges, resonant wires, etc.

Further, the disclosed embodiments are applicable for surveillance of all types of extracorporeal blood flow circuits in which blood is taken from a patient's circulation to have a process applied to it before it is returned to the circulation. Such blood flow circuits include hemodialysis, hemofiltration, hemodiafiltration, plasmapheresis, apheresis, extracorporeal membrane oxygenation, assisted blood circulation, and extracorporeal liver support/dialysis.

Further, the inventive monitoring techniques are applicable to any type of pumping device that generates pressure pulses in the first fluid containing system, not only rotary peristaltic pumps as disclosed above, but also other types of positive displacement pumps, such as linear peristaltic pumps, diaphragm pumps, as well as centrifugal pumps.

Still further, the inventive monitoring techniques are applicable also for monitoring the integrity of the fluid connection between the blood vessel access and the arterial needle based on a measurement signal from one or more arterial pressure sensors. Such a monitoring technique may provide a faster detection of malfunction than the conventional air detector, and more reliable detection of malfunction than conventional comparison of average pressure levels to threshold values. In such an application, the aforesaid reference signal may be derived from one or more venous pressure sensors in the extracorporeal blood flow circuit.

Also, it is to be understood that the monitoring technique is equally applicable to single-needle dialysis.

The inventive monitoring techniques are also applicable when the measurement signal originates from a pressure sensor arranged to sense the pressure in the human blood system. In such an embodiment, the first fluid containing system (S1) is the human blood system, the second fluid containing system (S2) is the extracorporeal blood flow circuit, and the fluid connection (C) may be formed by a connection between an access device and a blood vessel access. The first pulses thus originate from the human heart, and the second pulses originate from the pumping device in the extracorporeal blood flow circuit (and/or any other pulse generator within or associated with the extracorporeal blood flow circuit), and the integrity of the fluid connection is determined by applying the first and/or second inventive concepts to detect the presence/absence of the second pulses in the measurement signal.

The above-described inventive concepts may also be applicable to monitoring the integrity of fluid connections for transferring other liquids than blood. Likewise, the fluid connections need not be provided in relation to a human, but could be provided in relation to any other type of fluid containing system.

In one example, the fluid connection is provided between a blood processing circuit and a container/machine, wherein blood is pumped from one container/machine through a blood processing device in the blood processing circuit and back to the container/machine, or to another container/machine downstream of the blood processing device. The blood processing device could be any known device configured to modify and/or analyse the blood.

In a further example, the fluid connection is provided between a dialyser and a reprocessing system, which reprocesses the dialyser by pumping water, optionally together with suitable chemicals through the dialyser. An example of a dialyser reprocessing system is known from US2005/0051472.

In another example, the fluid connection is provided between a dialysate supply and a dialysate regeneration system, which circulates dialysate from the dialysate supply through a dialysate regeneration device and back to the supply. An example of a dialysate regeneration device is known from WO 05/062973.

In yet another example, the fluid connection is provided in an arrangement for priming an extracorporeal blood flow circuit by pumping a priming fluid from a supply via the blood flow circuit to a dialyser. The priming fluid may e.g. be dialysis solution, saline, purified water, etc.

In a still further example, the fluid connection is provided in an arrangement for cleaning and disinfecting the dialysis solution flow path of a dialysis machine, which pumps a cleaning fluid via a flow path to a dialyser/dialyser tubing. The cleaning fluid may e.g. be hot water, a chemical solution, etc.

In a further example, the fluid connection is provided in an arrangement for purifying water, which pumps water from a supply through a purifying device. The purifying device may use any known water purification technique, e.g. reverse osmosis, deionization or carbon absorption.

In another example, the fluid connection is provided in an arrangement for providing purified water to a dialysis machine, e.g. to be used in the preparation of dialysis solution therein.

In all of these examples, and in other applications related to medical treatment of human or animal patients, it may be vital to monitor the integrity of the fluid connection. Such monitoring can be accomplished according to the inventive concepts disclosed herein.

End Appendix B

1. A device for monitoring a fluid flow rate (Q) of a cardiovascular system of a mammalian subject, said device comprising:
an input receiving device for obtaining a time-dependent measurement signal (d(n)) from a pressure sensor in an extracorporeal blood circuit which is configured to connect to the cardiovascular system, the pressure sensor being arranged to detect a subject pulse originating from a subject pulse generator in the cardiovascular system of the subject,
a signal processor connected to the input receiving device and being configured to:
process the measurement signal to obtain a pulse profile (e(n)) which is a temporal signal profile of the subject pulse, and
calculate a fluid flow rate (Q) based at least partly on the temporal signal profile.

2. The device according to claim 1, wherein the subject pulse generator is a part of the cardiovascular system.

3. The device according to claim 2, wherein the subject pulse generator is at least one of the heart, and the breathing system of the subject.

4. The device according to claim 1, wherein the extracorporeal blood circuit comprises a fluid pathway, a blood processing device, and at least one pumping device, and wherein the pressure sensor is further configured to detect a pump pulse originating from the pumping device.

5. The device according to claim 1, wherein the calculation of the fluid flow rate (Q) includes calculating one or more of amplitude, shape, and timing of the temporal signal profile.

6. A method for monitoring a fluid flow rate (Q) in a cardiovascular system of a mammalian subject, said method comprising:
obtaining a time-dependent measurement signal (d(n)) from a pressure sensor in an extracorporeal blood circuit which is arranged in fluid connection with the cardiovascular system, the pressure sensor being arranged to detect a subject pulse originating from a subject pulse generator,
processing the measurement signal to obtain a pulse profile (e(n)) which is a temporal signal profile of the subject pulse, and
calculating a fluid flow rate (Q) based at least partly on the temporal signal profile.

7. The method according to claim 6, further comprising varying a blood flow of the extracorporeal blood circuit.

8. The method according to claim 6, further comprising aggregating a plurality of pulse profiles within an aggregation time window in the measurement signal and calculating the fluid flow rate (Q) based on an average of the plurality of the pulse profiles.

9. The method according to claim 6, wherein the extracorporeal blood circuit comprises a fluid pathway, a blood processing device, and at least one pumping device, and wherein the method further comprises detecting a pump pulse originating from the pumping device.

10. The method according to claim 6, wherein the calculating involves calculation of the cardiac output (CO) of the cardiovascular system.

11. The method according to claim 6, wherein the calculating involves calculation of an access flow (Qa) of a blood access in the cardiovascular system.

12. The method according to claim 6, further comprising calibrating the fluid flow rate (Q) against one or more calibration values.

13. The method according to claim 12, wherein the calibration comprises:
providing a detectable perturbation to at least a measurable blood characteristic in the cardiovascular system;
measuring an integrated change of a corresponding characteristic on a treatment fluid outlet of the extracorporeal blood circuit; and
determining the fluid flow rate (Q) based on the measurement of said integrated change of the treatment fluid outlet.

14. The method according to claim 12, wherein the calibration comprises:
obtaining a first conductivity or concentration measurement in a treatment fluid of the extracorporeal blood circuit running in a first direction;
obtaining a second conductivity or concentration measurement in the treatment fluid running in a second direction; and
calculating the access flow rate (Qa) in said blood access as a function of said first conductivity or concentration measurement and of said second conductivity or concentration measurement.

15. The method according to claim 6, further comprising calculating an average access flow rate (Qa) and an associated variance (QaV), retrieving a withdrawal blood flow rate (Qb), and generating an alarm event if the sum of (Qb) and (QaV) exceeds (Qa).

16. The method according to claim 6, further comprising:
calculating at least one additional fluid flow rate (Qx);
calculating an average fluid flow rate (Qavg) determined from the calculated fluid flow rate (Q) and the at least one additional fluid flow rate (Qx);
calculating an average reference fluid flow rate (Qavg_ref); and
adjusting the average reference fluid flow rate (Qavt_ref) based on the fluid flow rate (Q) and the at least one additional fluid flow rate (Qx).

17. The method according to claim 6, further comprising calculating a reference fluid flow rate (Qref) when the calculated fluid flow rate (Q) corresponds to an average fluid flow rate.

18. The method according to claim 10, wherein an alarm event is generated when the Cardiac Output (CO) exceeds a predetermined threshold.

19. The method according to claim 6, further comprising:
defining an initial model (Mo);
assigning the initial model (Mo) to a current model (CM);
generating a parameter (P) that correlates with the fluid flow rate Q;
acquiring flow calibration data (C);
investigating whether a model validity criterion (MVC) is fulfilled or not by comparing parameter (P), calibration data (C) with the current model (CM), wherein in case the model validity criterion (MVC) is not fulfilled then repeatedly generating a new model (NM) and assigning the current model (CM) with new model (NM) until model validity criterion (MVC) is fulfilled; and
calculating a fluid flow rate (Q) based at least partly on the temporal signal profile, if the model validity criterion MVC is fulfilled.

20. The method according to claim 19, further comprising one or more of acquiring blood pressure (BP) of the subject and comparing said blood pressure (BP) with the current model (CM); and
storing of current model (CM) and available parameters (M, C, BP, P, TD, PD).

21. The method according to claim 6, wherein the calculating of a fluid flow rate (Q) involves a pulse parameter P from one or more of amplitude, shape, and timing of the temporal signal profile.

22. A computer-readable medium comprising computer instructions which, when executed by a processor, cause the processor to perform the method of claim 6.

23. A device for monitoring a fluid flow rate (Q) of a cardiovascular system of a subject, said device comprising:
means for obtaining a time-dependent measurement signal (d(n)) from a pressure sensor in an extracorporeal blood circuit which is adapted for connection to the cardiovascular system, the pressure sensor being arranged to detect a subject pulse originating from a subject pulse generator in the cardiovascular system of the subject,
means for processing the measurement signal to obtain a pulse profile (e(n)) which is a temporal signal profile of the subject pulse, and
means for calculating a fluid flow rate (Q) based at least partly on the temporal signal profile.

24. A system configured to calculate a flow rate of blood in a cardiovascular system of a mammal, the system comprising a non-transitory memory and a signal processor executing instructions stored in the memory, the instructions cause the system to:
receive a time-dependent measurement signal from a pressure sensor monitoring blood flow through an extracorporeal blood circuit configured to receive blood withdrawn from the cardiovascular system, treat the blood and infuse the treated blood to the cardiovascular system, the pressure sensor generating data representative of pressure pulses in the blood flow;
generate a temporal signal profile of at least one of the pressure pulses based on the measurement signal, and
calculate a flow rate of the received blood based on the temporal signal profile.
